(19) United States
(12) Patent Application Publication
(10) Pub. No.: US 2025/0043310 A1
(43) Pub. Date: Feb. 6, 2025

(54) GENE THERAPY OF HEMOPHILIA A USING VIRAL VECTORS ENCODING RECOMBINANT FVIII VARIANTS WITH INCREASED EXPRESSION

(71) Applicant: Takeda Pharmaceutical Company Limited, Osaka (JP)

(72) Inventors: Hanspeter Rottensteiner, Vienna (AT); Werner Hoellriegl, Altenmarkt/Triesting (AT)

(21) Appl. No.: 18/797,893

(22) Filed: Aug. 8, 2024

Related U.S. Application Data
(63) Continuation of application No. 17/296,944, filed on May 25, 2021, now Pat. No. 12,091,675.
(60) Provisional application No. 62/867,171, filed on Jun. 26, 2019.
(60) Provisional application No. 62/698,680, filed on Jul. 16, 2018.

(51) Int. Cl. C12N 15/861; A61K 38/37; A61K 48/00; C07K 14/755

(52) U.S. Cl. CPC C12N 15/861; A61K 38/37; A61K 48/0083; C07K 14/755; A01K 2217/075; A01K 2227/105; A01K 2267/0381

(57) ABSTRACT

The present disclosure provides, among other aspects, codon-altered polynucleotides encoding Factor VIII variants for expression in mammalian cells. In some embodiments, the disclosure also provides mammalian gene therapy vectors and methods for treating hemophilia A. In some embodiments, the present disclosure provides methods for dosing a hemophilia A patient with a polynucleotide, e.g., a codon-altered polynucleotide, encoding a Factor VII polypeptide.

### CROSS REFERENCE TO RELATED APPLICATIONS

This application is a Continuation of U.S. application Ser. No. 17/296,944, filed May 25, 2021, which claims priority to PCT Application No. PCT/US19/41802, filed Jul. 15, 2019, which claims priority to U.S. Provisional Patent Application Ser. No. 62/698,680, filed Jul. 16, 2018, and claims priority to U.S. Provisional Patent Application Ser. No. 62/867,171, filed Jun. 26, 2019, all of which are hereby incorporated by reference in their entireties.

### SEQUENCE LISTING

The instant application contains a Sequence Listing which has been submitted electronically in XML format and is hereby incorporated by reference in its entirety. Said XML copy, created on Aug. 6, 2024, is named 008073-5202-US01_Sequence_Listing.xml and is 60 kilobytes in size.

### BACKGROUND OF THE DISCLOSURE

Blood coagulation proceeds through a complex and dynamic biological pathway of interdependent biochemical reactions, referred to as the coagulation cascade. Coagulation Factor VIII (FVIII) is a key component in the cascade. Factor VIII is recruited to bleeding sites, and forms a Xase complex with activated Factor IX (FIXa) and Factor X (FX). The Xase complex activates FX, which in turn activates prothrombin to thrombin, which then activates other components in the coagulation cascade to generate a stable clot (reviewed in Saenko et al., Trends Cardiovasc. Med., 9:185-192 (1999); Lenting et al., Blood, 92:3983-3996 (1998)).

Hemophilia A is a congenital X-linked bleeding disorder characterized by a deficiency in Factor VIII activity. Diminished Factor VIII activity inhibits a positive feedback loop in the coagulation cascade. This causes incomplete coagulation, which manifests as bleeding episodes with increased duration, extensive bruising, spontaneous oral and nasal bleeding, joint stiffness and chronic pain, and possibly internal bleeding and anemia in severe cases (Zhang et al., Clinic. Rev. Allerg. Immunol., 37:114-124 (2009)).

Conventionally, hemophilia A is treated by Factor VIII replacement therapy, which consists of administering Factor VIII protein (e.g., plasma-derived or recombinantly-produced Factor VIII) to an individual with hemophilia A. Factor VIII is administered prophylactically to prevent or reduce frequency of bleeding episodes, in response to an acute bleeding episode, and/or perioperatively to manage bleeding during surgery. However, there are several undesirable features of Factor VIII replacement therapy.

First, Factor VIII replacement therapy is used to treat or manage hemophilia A, but does not cure the underlying Factor VIII deficiency. Because of this, individuals with hemophilia A require Factor VIII replacement therapy for the duration of their lives. Continuous treatment is expensive and requires the individual to maintain strict compliance, as missing only a few prophylactic doses can have serious consequences for individuals with severe hemophilia A.

Second, because Factor VIII has a relatively short half-life in vivo, conventional prophylactic Factor VIII replacement therapy requires administration every second or third day. This places a burden on the individual to maintain compliance throughout their life. While third generation “long-acting” Factor VIII drugs may reduce the frequency of administration, prophylactic Factor FVIII replacement therapy with these drugs still requires monthly, weekly, or more frequent administration in perpetuity. For example, prophylactic treatment with ELOCTATE™ [Antihemophilic Factor (Recombinant), Fc Fusion Protein] requires administration every three to five days (ELOCTATE™ Prescribing Information, Biogen Idec Inc., (2015)). Moreover, the long-term effects of chemically modified biologics (e.g., pegylated polypeptides) are not yet fully understood.

Third, between 15% and 30% of all individuals receiving Factor VIII replacement therapy form anti-Factor VIII inhibitor antibodies, rendering the therapy inefficient. Factor VIII bypass therapy (e.g., administration of plasma-derived or recombinantly-produced prothrombin complex concentrates) can be used to treat hemophilia in individuals that form inhibitor antibodies. However, Factor VIII bypass therapy is less effective than Factor VIII replacement therapy (Mannucci P.M., J Thromb Haemost., 1(7):1349-55 (2003)) and may be associated with an increased risk of cardiovascular complication (Luu and Ewenstein, Haemophilia, 10 Suppl. 2:10-16 (2004)).

Somatic gene therapy holds great promise for the treatment of hemophilia A because it would remedy the underlying under-expression functional Factor VIII activity (e.g., due to missense or nonsense mutations), rather than provide a one-time dose of Factor VIII activity to the individual. Because of this difference in the mechanism of action, as compared to Factor VIII replacement therapy, one-time administration of a Factor VIII gene therapy vector may provide an individual with Factor VIII for several years, reducing the cost of treatment and eliminating the need for continued patient compliance.

Coagulation Factor IX (FIX) gene therapy has been used effectively to treat individuals with hemophilia B, a related blood coagulation condition characterized by diminished Factor IX activity (Manno C.S., et al., Nat Med., 12(3):342-47 (2006)). However, Factor VIII gene therapy presents several unique challenges. For example, the full-length, wild-type Factor VIII polypeptide (2351 amino acids; UniProt accession number P00451) is five times larger than the full-length, wild-type Factor IX polypeptide (461 amino acids; UniProt accession number P00740). As such, the coding sequence of wild-type Factor VIII is 7053 base pairs, which is too large to be packaged in conventional AAV gene therapy vectors. Further, reported recombinant expression of B-domain deleted variants of Factor VIII (BDD-FVIII) has been poor. As such, several groups have attempted to alter the codon usage of BDD-FVIII constructs, with limited success.

### BRIEF SUMMARY OF DISCLOSURE

Accordingly, there is a need for Factor VIII variants whose coding sequences are more efficiently packaged into, and delivered via, gene therapy vectors. There is also a need for synthetic, codon-altered nucleic acids which express Factor VIII more efficiently. Such Factor VIII variants and codon-altered nucleic acids allow for improved treatment of Factor VIII deficiencies (e.g., hemophilia A). The above deficiencies and other problems associated with the treatment of Factor VIII deficiencies (e.g., hemophilia A) are reduced or eliminated by the disclosed codon-altered Factor VIII variants.

In accordance with some embodiments, the present disclosure provides nucleic acids encoding Factor VIII variants that have high sequence identity to the disclosed codon-altered sequences of the Factor VIII heavy chain (e.g., CS04-HC-NA) and light chain (e.g., CS04-LC-NA). In some embodiments, these nucleic acids further include a sequence encoding a linker sequence that replaces the native Factor VIII B-domain (e.g., a linker sequences comprising a furin cleavage site), between the sequences coding for the Factor VIII heavy and light chains.

In one aspect, the disclosure provides a polynucleotide including a nucleotide sequence encoding a Factor VIII polypeptide. The Factor VIII polypeptide includes a light chain, a heavy chain, and a polypeptide linker joining the C-terminus of the heavy chain to the N-terminus of the light chain. The heavy chain of the Factor VIII polypeptide is encoded by a first nucleotide sequence having at least 95% identity to CS04-HC-NA (SEQ ID NO: 3). The light chain of the Factor FVIII polypeptide is encoded by a second nucleotide sequence having at least 95% identity to CS04-LC-NA (SEQ ID NO: 4). The polypeptide linker comprises a furin cleavage site.

In one embodiment of the polynucleotides described above, the polypeptide linker is encoded by a third nucleotide sequence having at least 95% identity to BDLO04 (SEQ ID NO: 5).

In one embodiment of the polynucleotides described above, the first nucleotide sequence encoding the heavy chain of the Factor VIII polypeptide has at least 96% identity to the respective heavy chain sequence (e.g., CS04-HC-NA (SEQ ID NO: 3)), and the second nucleotide sequence encoding the light chain of the Factor FVIII polypeptide has at least 96% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide has at least 97% identity to the respective heavy chain sequence (e.g., CS04-HC-NA (SEQ ID NO: 3)), and the second nucleotide sequence encoding the light chain of the Factor FVIII polypeptide has at least 97% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide has at least 98% identity to the respective heavy chain sequence (e.g., CS04-HC-NA (SEQ ID NO: 3)), and the second nucleotide sequence encoding the light chain of the Factor FVIII polypeptide has at least 98% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide has at least 99% identity to the respective heavy chain sequence (e.g., CS04-HC-NA (SEQ ID NO: 3)), and the second nucleotide sequence encoding the light chain of the Factor FVIII polypeptide has at least 99% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide has at least 99.5% identity to the respective heavy chain sequence (e.g., CS04-HC-NA (SEQ ID NO: 3)), and the second nucleotide sequence encoding the light chain of the Factor FVIII polypeptide has at least 99.5% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide has at least 99.9% identity to the respective heavy chain sequence (e.g., CS04-HC-NA (SEQ ID NO: 3)), and the second nucleotide sequence encoding the light chain of the Factor FVIII polypeptide has at least 99.9% identity to the respective light chain sequence (e.g., CS04-LC-NA (SEQ ID NO: 4)).

In one embodiment of the polynucleotides described above, the first nucleotide sequence encoding the heavy chain of the Factor VIII polypeptide is CS04-HC-NA (SEQ ID NO: 3), and the second nucleotide sequence encoding the light chain of the Factor FVIII polypeptide is CS04-LC-NA (SEQ ID NO: 4).

In one aspect, the disclosure provides a polynucleotide comprising a nucleotide sequence having at least 95% identity to CS04-FL-NA, wherein the polynucleotide encodes a Factor VIII polypeptide.

In one embodiment of the polynucleotides described above, the nucleotide sequence has at least 96% identity to the respective full-length polynucleotide sequence (e.g., CS04-FL-NA (SEQ ID NO: 1)).

In one embodiment of the polynucleotides described above, the nucleotide sequence has at least 97% identity to the respective full-length polynucleotide sequence (e.g., CS04-FL-NA (SEQ ID NO: 1)).

In one embodiment of the polynucleotides described above, the nucleotide sequence has at least 98% identity to the respective full-length polynucleotide sequence (e.g., CS04-FL-NA (SEQ ID NO: 1))

In one embodiment of the polynucleotides described above, the nucleotide sequence has at least 99% identity to the respective full-length polynucleotide sequence (e.g., CS04-FL-NA (SEQ ID NO: 1)).

In one embodiment of the polynucleotides described above, the nucleotide sequence has at least 99.5% identity to the respective full-length polynucleotide sequence (e.g., CS04-FL-NA (SEQ ID NO: 1)).

In one embodiment of the polynucleotides described above, the nucleotide sequence has at least 99.9% identity to the respective full-length polynucleotide sequence (e.g., CS04-FL-NA (SEQ ID NO: 1)).

In one embodiment of the polynucleotides described above, the nucleotide sequence is CS04-FL-NA (SEQ ID NO: 1).

In one embodiment of the polynucleotides described above, the polynucleotide encodes a Factor VIII polypeptide comprising an amino acid sequence having at least 95% identity to CS04-FL-AA (SEQ ID NO: 2).

In one embodiment of the polynucleotides described above, the polynucleotide encodes a Factor VIII polypeptide comprising an amino acid sequence having at least 96% identity to CS04-FL-AA (SEQ ID NO: 2).

In one embodiment of the polynucleotides described above, the polynucleotide encodes a Factor VIII polypeptide comprising an amino acid sequence having at least 97% identity to CS04-FL-AA (SEQ ID NO: 2).

In one embodiment of the polynucleotides described above, the polynucleotide encodes a Factor VIII polypeptide comprising an amino acid sequence having at least 98% identity to CS04-FL-AA (SEQ ID NO: 2).

In one embodiment of the polynucleotides described above, the polynucleotide encodes a Factor VIII polypeptide comprising an amino acid sequence having at least 99% identity to CS04-FL-AA (SEQ ID NO: 2).

In one embodiment of the polynucleotides described above, the polynucleotide encodes a Factor VIII polypeptide comprising an amino acid sequence having at least 99.5% identity to CS04-FL-AA (SEQ ID NO: 2).

In one embodiment of the polynucleotides described above, the polynucleotide encodes a Factor VIII polypeptide comprising an amino acid sequence having at least 99.9% identity to CS04-FL-AA (SEQ ID NO: 2).

In one embodiment of the polynucleotides described above, the polynucleotide encodes a Factor VIII polypeptide comprising the amino acid sequence of CS04-FL-AA (SEQ ID NO: 2).

In one embodiment of the polynucleotides described above, the nucleotide sequence has at least 95% identity to a sequence selected from the group consisting of CS04-FL-NA, CS04-HC-NA, and CS04-LC-NA.

In one embodiment of the polynucleotides described above, the nucleotide sequence has at least 96% identity to a sequence selected from the group consisting of CS04-FL-NA, CS04-HC-NA, and CS04-LC-NA.

In one embodiment of the polynucleotides described above, the nucleotide sequence has at least 97% identity to a sequence selected from the group consisting of CS04-FL-NA, CS04-HC-NA, and CS04-LC-NA.

In one embodiment of the polynucleotides described above, the nucleotide sequence has at least 98% identity to a sequence selected from the group consisting of CS04-FL-NA, CS04-HC-NA, and CS04-LC-NA.

In one embodiment of the polynucleotides described above, the nucleotide sequence has at least 99% identity to a sequence selected from the group consisting of CS04-FL-NA, CS04-HC-NA, and CS04-LC-NA.

In one embodiment of the polynucleotides described above, the nucleotide sequence has at least 99.5% identity to a sequence selected from the group consisting of CS04-FL-NA, CS04-HC-NA, and CS04-LC-NA.

In one embodiment of the polynucleotides described above, the nucleotide sequence has at least 99.5% identity to a sequence selected from the group consisting of CS04-FL-NA, CS04-HC-NA, and CS04-LC-NA.

In one embodiment of the polynucleotides described above, the nucleotide sequence is selected from the group consisting of CS04-FL-NA, CS04-HC-NA, and CS04-LC-NA.

In one embodiment of the polynucleotides described above, the polynucleotide also includes a promoter element operably linked to the polynucleotide encoding the Factor VIII polypeptide.

In one embodiment of the polynucleotides described above, the polynucleotide also includes an enhancer element operably linked to the polynucleotide encoding the Factor VIII polypeptide.

In one embodiment of the polynucleotides described above, the polynucleotide also includes a polyadenylation element operably linked to the polynucleotide encoding the Factor VIII polypeptide.

In one embodiment of the polynucleotides described above, the polynucleotide also includes an intron operatively linked to the nucleotide sequence encoding the Factor VIII polypeptide.

In one embodiment of the polynucleotides described above, the intron is positioned between a promoter element and the translation initiation site (e.g., the first coding ATG) of the nucleotide sequence encoding a Factor VIII polypeptide.

In another aspect, the disclosure provides a mammalian gene therapy vector including a polynucleotide as described above.

In one embodiment of the mammalian gene therapy vector described above, the mammalian gene therapy vector is an adeno-associated virus (AAV) vector.

In one embodiment of the mammalian gene therapy vector described above, the AAV vector is an AAV-8 vector.

In another aspect, the disclosure provides a method for treating hemophilia A including administering, to a patient in need thereof, a mammalian gene therapy vector as described above.

In another aspect, the disclosure provides a mammalian gene therapy vector as described above for treating hemophilia A.

In another aspect, the disclosure provides the use of a mammalian gene therapy vector as described above for the manufacture of a medicament for treating hemophilia A.

### BRIEF DESCRIPTION OF DRAWINGS

FIG. 1 shows schematic illustrations of the wild-type and ReFacto-type human Factor VIII protein constructs.

FIGS. 2A and 2B show the CS04 codon-altered nucleotide sequence (SEQ ID NO: 1) encoding a Factor VIII variant in accordance with some embodiments (“CS04-FL-NA” for full-length coding sequence).

FIG. 3 shows the Factor VIII variant amino acid sequence (SEQ ID NO: 2) encoded by the CS04 codon-altered nucleotide sequence in accordance with some embodiments (“CS04-FL-AA” for full-length amino acid sequence).

FIG. 4 shows the portion of the CS04 codon-altered nucleotide sequence (SEQ ID NO: 3) encoding the heavy chain of a Factor VIII variant in accordance with some embodiments (“CS04-HC-NA”).

FIG. 5 shows the portion of the CS04 codon-altered nucleotide sequence (SEQ ID NO: 4) encoding the light chain of a Factor VIII variant in accordance with some embodiments (“CS04-LC-NA”).

FIG. 6 shows an exemplary coding sequence (SEQ ID NO: 5) for a B-domain substituted linker in accordance with some embodiments. BDLO04 (SEQ ID NO: 5) is the respective portion of the CS04 codon-altered nucleotide sequence that encodes a B-domain substituted linker.

FIGS. 7A, 7B, and 7C show an AAV vector sequence (SEQ ID NO: 6) containing an CS04 codon-altered nucleotide sequence in accordance with some embodiments (“CS04-AV-NA”).

FIGS. 8A and 8B show the CS08 codon-altered nucleotide sequence (SEQ ID NO: 7) encoding a Factor VIII variant in accordance with some embodiments (“CS08-FL-NA”).

FIGS. 9A and 9B show the CS10 codon-altered nucleotide sequence (SEQ ID NO: 8) encoding a Factor VIII variant in accordance with some embodiments (“CS10-FL-NA”).

FIGS. 10A and 10B show the CS11 codon-altered nucleotide sequence (SEQ ID NO: 9) encoding a Factor VIII variant in accordance with some embodiments (“CS11-FL-NA”).

FIGS. 11A and 11B show the CS40 wild-type ReFacto coding sequence (SEQ ID NO: 10), in accordance with some embodiments (“CS40-FL-NA”).

FIGS. 12A and 12B show the CH25 codon-altered nucleotide sequence (SEQ ID NO: 11) encoding a Factor VIII variant in accordance with some embodiments (“CH25-FL-NA”).

FIG. 13 shows a wild-type human Factor VIII amino acid sequence (SEQ ID NO: 12), in accordance with some embodiments (“FVIII-FL-AA”).

FIG. 14 illustrates the scheme for cloning the pCS40, pCS04, pCS08, pCS10, pCS11, and pCh25 constructs, by inserting synthetic Refacto-type BDD-FVIII DNA sequences into the vector backbone pCh-BB01 via AscI and NotI restriction sites.

FIG. 15 shows the integrity of AAV vector genome preparations, as analyzed by agarose gel electrophoresis. Lane 1, DNA marker; lane 2, vCS40; lane 4, vCS04. The AAV vectors have all the same-sized genomes, migrating at approximately 5 kb (arrow, right side). The scale on the left side indicates size of the DNA fragments in kilobases (kb).

FIG. 16 shows the protein analysis of AAV vector preparations by PAGE and silver staining. Lane 1, protein marker (M); lane 2, vCS40; and lane 4, vCS04. The constructs all have the same AAV8 capsids consisting of VP1, VP2, and VP3 (arrows right side). The scale on the left side indicates size of the protein marker in kilodaltons (kDa).

FIG. 17 shows FVIII activity following systemic administration of a (r) AAV8-based gene therapy vector containing the CS04 Factor VIII codon optimized construct, as described in Example 3. cp, vector capsid particles; FVIII, factor VIII; LLOQ, lower limit of quantification. 14, 28, 42, and 56-day time points are shown left to right in the graph.

FIG. 18 shows reduced blood loss, in a tail-tip bleeding assay, after systemic administration of a (r) AAV8-based gene therapy vector containing the CS04 Factor VIII codon optimized construct, as described in Example 3. cp, vector capsid particles.

FIGS. 19A, 19B, and 19C show biodistribution of the (r) AAV8-based gene therapy vector containing the CS04 Factor VIII codon optimized construct DNA after systemic administration. 1902=liver; 1904=lymph node; 1906=skeletal muscle; 1908=heart; 1910=kidney; 1912=spleen; 1914=lung; 1916=testis; 1918=brain.

### DETAILED DESCRIPTION OF DISCLOSURE

### I. INTRODUCTION

AAV-based gene therapy holds great promise for the treatment of hemophiliacs. For hemophilia B, first clinical data are encouraging in that FIX levels of about 10% can be maintained in at least some patients for more than 1 year. For hemophilia A however, achieving therapeutic expression levels of 5-10% with AAV vectors remains challenging for various reasons. First, the Factor VIII coding sequence is too large for conventional AAV-based vectors. Second, engineered B-domain deleted or truncated Factor VIII constructs suffer from poor expression in vivo, even when codon-optimized. Third, these B-domain deleted or truncated Factor VIII variant constructs have short half-lives in vivo, exacerbating the effects of poor expression. Fourth, even when expressed, FVIII is not efficiently secreted from cells, as are other coagulation factors, such as Factor IX. Therefore, strategies to improve the expression of FVIII are needed to make FVIII gene therapy a viable therapeutic option for hemophilia A patients.

The present disclosure relates to the discovery of codon-altered Factor VIII variant coding sequences that solve these and other problems associated with Factor VIII gene therapy. For example, the polynucleotides disclosed herein provide markedly improved expression in mammalian cells, and display improved virion packaging due to stabilized packing interactions. In some implementations, these advantages are realized by using coding sequences for the heavy and light chains of Factor VIII with high sequence identity to the codon altered CS04 construct (e.g., with high sequence identity to the CS04-HC heavy chain coding sequence and high sequence identity to the CS04-LC light chain coding sequence).

In some implementations, the Factor VIII molecules encoded by the polynucleotides described herein have been shortened by truncating, deleting, or replacing the wild-type B-domain. As such, the polynucleotides are better suited for expressing Factor VIII via conventional gene therapy vectors, which inefficiently express larger polypeptides, such as the wild-type Factor VIII.

Advantageously, it is shown herein that the CS04 codon-altered Factor VIII variant coding sequence provide superior expression of a B-domain deleted Factor VIII construct in vivo. For example, it is demonstrated in Example 2 and Table 4 that intravenous administration of AAV-based gene therapy vectors having the CS04 (SEQ ID NO: 1) coding sequence provides a 74-fold increase in Factor VIII expression, relative to the corresponding CS40 construct encoded with the wild-type polynucleotide sequence (SEQ ID NO: 10), in Factor VIII knock-out mice (Table 4).

Further, it also shown herein that the CS04 codon-altered Factor VIII variant coding sequence provides superior virion packaging and virus production. For example, it is demonstrated in Example 1 that AAV vector constructs containing the CS04 construct provided 5 to 7-fold greater viral yield, relative to the corresponding CS40 construct encoded with the wild-type polynucleotide sequence, when isolated from the same amount of cell pellet.

### II. DEFINITIONS

As used herein, the following terms have the meanings ascribed to them unless specified otherwise.

As used herein, the terms “Factor VIII” and “FVIII” are used interchangeably, and refer to any protein with Factor VIII activity (e.g., active FVIII, often referred to as FVIIIa) or protein precursor (e.g., pro-protein or pre-pro-protein) of a protein with Factor VIII activity, particularly Factor IXa cofactor activity. In an exemplary embodiment, a Factor VIII polypeptide refers to a polypeptide that has sequences with high sequence identity (e.g., at least 70%, 75%, 80%, 85%, 90%, 95%, 99%, or more) to the heavy and light chains of a wild type Factor VIII polypeptide. In some embodiments, the B-domain of a Factor VIII polypeptide is deleted, truncated, or replaced with a linker polypeptide to reduce the size of the polynucleotide encoding the Factor VIII polypeptide. In an exemplary embodiment, amino acids 20-1457 of CS04-FL-AA constitute a Factor VIII polypeptide.

Non-limiting examples of wild type Factor VIII polypeptides include human pre-pro-Factor VIII (e.g., GenBank accession nos. AAA52485, CAA25619, AAA58466, AAA52484, AAA52420, AAV85964, BAF82636, BAG36452, CAI41660, CAI41666, CAI41672, CAI43241, CAO03404, EAW72645, AAH22513, AAH64380, AAH98389, AAI11968, AAI11970, or AAB61261), corresponding pro-Factor VIII, and natural variants thereof; porcine pre-pro-Factor VIII (e.g., UniProt accession nos. FIRZ36 or K7GSZ5), corresponding pro-Factor VIII, and natural variants thereof; mouse pre-pro-Factor VIII (e.g., GenBank accession nos. AAA37385, CAM15581, CAM26492, or EDL29229), corresponding pro-Factor VIII, and natural variants thereof; rat pre-pro-Factor VIII (e.g., GenBank accession no. AAQ21580), corresponding pro-Factor VIII, and natural variants thereof; rat pre-pro-Factor VIII; and other mammalian Factor VIII homologues (e.g., monkey, ape, hamster, guinea pig, etc.).

As used herein, a Factor VIII polypeptide includes natural variants and artificial constructs with Factor IX cofactor activity. As used in the present disclosure, Factor VIII encompasses any natural variants, alternative sequences, isoforms, or mutant proteins that retain some basal Factor IX cofactor activity (e.g., at least 5%, 10%, 25%, 50%, 75%, or more of the corresponding wild type activity). Examples of Factor VIII amino acid variations (relative to FVIII-FL-AA (SEQ ID NO: 12)) found in the human population include, without limitation, S19R, R22T, Y24C, Y25C, L26P/R, E30V, W33G, Y35C/H, G41C, R48C/K, K67E/N, L69P, E72K, D75E/V/Y, P83R, G89D/V, G92A/V, A97P, E98K, V99D, D101G/H/V, V104D, K108T, M110V, A111T/V, H113R/Y, L117F/R, G121S, E129V, G130R, E132D, Y133C, D135G/Y, T137A/I, S138R, E141K, D145H, V147D, Y155H, V159A, N163K, G164D/V, P165S, C172W, S176P, S179P, V181E/M, K185T, D186G/N/Y, S189L, L191F, G193R, L195P, C198G, S202N/R, F214V, L217H, A219D/T, V220G, D222V, E223K, G224W, T252I, V253F, N254I, G255V, L261P, P262L, G263S, G266F, C267Y, W274C, H275L, G278R, G280D, E284K, V285G, E291G/K, T294I, F295L, V297A, N299I, R301C/H/L, A303E/P, I307S, S308L, F312S, T314A/I, A315V, G323E, L326P, L327P/V, C329F, I331V, M339T, E340K, V345A/L, C348R/S/Y, Y365C, R391C/H/P, S392L/P, A394S, W401G, I405F/S, E409G, W412G/R, K427I, L431F/S, R437P/W, I438F, G439D/S/V, Y442C, K444R, Y450D/N, T454I, F455C, G466E, P470L/R/T, G474E/R/V, E475K, G477V, D478N, T479R, F484C, A488G, R490G, Y492C/H, Y492H, I494T, P496R, G498R, R503H, G513S/V, I522Y, K529E, W532G, P540T, T541S, D544N, R546W, R550C/G/H, S553P, S554C/G, V556D, R560T, D561G/H/Y, 1567T, P569R, S577F, V578A, D579A/H, N583S, Q584H/K/R, I585R/T, M586V, D588G/Y, L594Q, S596P, N601D/K, R602G, S603I/R, W604C, Y605H/S, N609I, R612C, N631K/S, M633I, S635N, N637D/I/S, Y639C, L644V, L650F, V653A/M, L659P, A663V, Q664P, F677L, M681I, V682F, Y683C/N, T686R, F698L, M699T/V, M701I, G705V, G710W, N713I, R717L/W, G720D/S, M721I/L, A723T, L725Q, V727F, E739K, Y742C, R795G, P947R, V1012L, E1057K, H1066Y, D1260E, K1289Q, Q1336K, N1460K, L1481P, A1610S, I1698T, Y1699C/F, E1701K, Q1705H, R1708C/H, T1714S, R1715G, A1720V, E1723K, D1727V, Y1728C, R1740G, K1751Q, F1762L, R1768H, G1769R, L1771P, L1775F/V, L1777P, G1779E/R, P1780L, I1782R, D1788H, M1791T, A1798P, S1799H, R1800C/G/H, P1801A, Y1802C, S1803Y, F1804S, L1808F, M1842I, P1844S, T1845P, E1848G, A1853T/V, S1858C, K1864E, D1865N/Y, H1867P/R, G1869D/V, G1872E, P1873R, L1875P, V1876L, C1877R/Y, L1882P, R1888I, E1894G, I1901F, E1904D/K, S1907C/R, W1908L, Y1909C, A1939T/V, N1941D/S, G1942A, M1945V, L1951F, R1960L/Q, L1963P, S1965I, M1966I/V, G1967D, S1968R, N1971T, H1973L, G1979V, H1980P/Y, F1982I, R1985Q, L1994P, Y1998C, G2000A, T2004R, M2007I, G2013R, W2015C, R2016P/W, E2018G, G2022D, G2028R, S2030N, V2035A, Y2036C, N2038S, 2040Y, G2045E/V, I2051S, I2056N, A2058P, W2065R, P2067L, A2070V, S2082N, S2088F, D2093G/Y, H2101D, T2105N, Q2106E/P/R, G2107S, R2109C, I2117F/S, Q2119R, F2120C/L, Y2124C, R2135P, S2138Y, T2141N, M2143V, F2145C, N2148S, N2157D, P2162L, R2169C/H, P2172L/Q/R, T2173A/I, H2174D, R2178C/H/L, R2182C/H/P, M2183R/V, L2185S/W, S2192I, C2193G, P2196R, G2198V, E2200D, I2204T, I2209N, A2211P, A2220P, P2224L, R2228G/L/P/Q, L2229F, V2242M, W2248C/S, V2251A/E, M2257V, T2264A, Q2265R, F2279C/I, I2281T, D2286G, W2290L, G2304V, D2307A, P2319L/S, R2323C/G/H/L, R2326G/L/P/Q, Q2330P, W2332R, 12336F, R2339T, G2344C/D/S, and C2345S/Y. Factor VIII proteins also include polypeptides containing post-translational modifications.

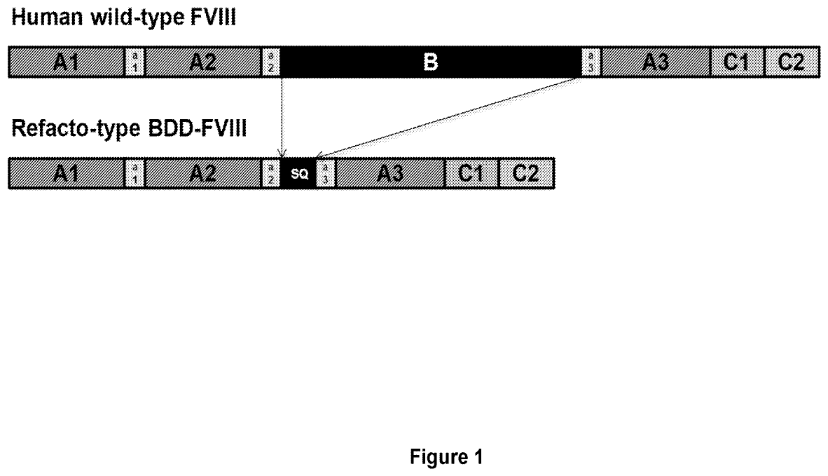

Generally, polynucleotides encoding Factor VIII encode for an inactive single-chain polypeptide (e.g., a pre-pro-protein) that undergoes post-translational processing to form an active Factor VIII protein (e.g., FVIIIa). For example, referring to FIG. 1, the wild type human Factor VIII pre-pro-protein is first cleaved to release the encoded signal peptide (not shown), forming a first single-chain pro-protein (shown as 037 human wild-type FVIII). The pro-protein is then cleaved between the B and A3 domains to form a first polypeptide that includes the Factor VIII heavy chain (e.g., the A1 and A2 domains) and B-domain, and a second polypeptide that includes the Factor VIII light chain (e.g., including the A3, C1, and C3 domains). The first polypeptide is further cleaved to remove the B-domain, and also to separate the A1 and A2 domains, which remain associated with the Factor VIII light chain in the mature Factor VIIIa protein. For review of the Factor VIII maturation process, see Graw et al., Nat Rev Genet., 6(6):488-501 (2005), the content of which is incorporated herein by reference in its entirety for all purposes.

However, in some embodiments, the Factor VIII polypeptide is a single-chain Factor VIII polypeptide. Single-chain Factor VIII polypeptides are engineered to remove natural cleavage sites, and optionally remove, truncate, or replace the B-domain of Factor VIII. As such, they are not matured by cleavage (other than cleavage of an optional signal and/or leader peptide), and are active as a single chain. Non-limiting examples of single-chain Factor VIII polypeptides are described in Zollner et al. (Thromb Res, 134(1):125-31 (2014)) and Donath et al. (Biochem J., 312(1):49-55 (1995)), the disclosures of which are hereby incorporated by reference in their entireties for all purposes.

As used herein, the terms “Factor VIII heavy chain,” or simply “heavy chain,” refers to the aggregate of the A1 and A2 domains of a Factor VIII polypeptide. In an exemplary embodiment, amino acids 20-759 of CS04-FL-AA (SEQ ID NO: 2) constitute a Factor VIII heavy chain.

As used herein, the term “Factor VIII light chain,” or simply “light chain,” refers to the aggregate of the A3, C1, and C2 domains of a Factor VIII polypeptide. In an exemplary embodiment, amino acids 774-1457 CS04-FL-AA (SEQ ID NO: 2) constitute a Factor VIII light chain. In some embodiments, a Factor VIII light chain excludes the acidic a3 peptide, which is released during maturation in vivo.

Generally, Factor VIII heavy and light chains are expressed as a single polypeptide chain, e.g., along with an optional B-domain or B-domain substituted linker. However, in some embodiments, a Factor VIII heavy chain and Factor VIII light chain are expressed as separate polypeptide chains (e.g., co-expressed), and reconstituted to form a Factor VIII protein (e.g., in vivo or in vitro).

As used herein, the terms “B-domain substituted linker” and “Factor VIII linker” are used interchangeably, and refer to truncated versions of a wild type Factor VIII B-domain (e.g., amino acids 760-1667 of FVIII-FL-AA (SEQ ID NO: 12)) or peptides engineered to replace the B-domain of a Factor VIII polypeptide. As used herein, a Factor VIII linker is positioned between the C-terminus of a Factor VIII heavy chain and the N-terminus of a Factor VIII light chain in a Factor VIII variant polypeptide in accordance with some embodiments. Non-limiting examples of B-domain substituted linkers are disclosed in U.S. Pat. Nos. 4,868,112, 5,112,950, 5,171,844, 5,543,502, 5,595,886, 5,610,278, 5,789,203, 5,972,885, 6,048,720, 6,060,447, 6,114,148, 6,228,620, 6,316,226, 6,346,513, 6,458,563, 6,924,365, 7,041,635, and 7,943,374; U.S. Patent Application Publication Nos. 2013/024960, 2015/0071883, and 2015/0158930; and PCT Publication Nos. WO 2014/064277 and WO 2014/127215, the disclosures of which are hereby incorporated by reference, in their entireties, for all purposes.

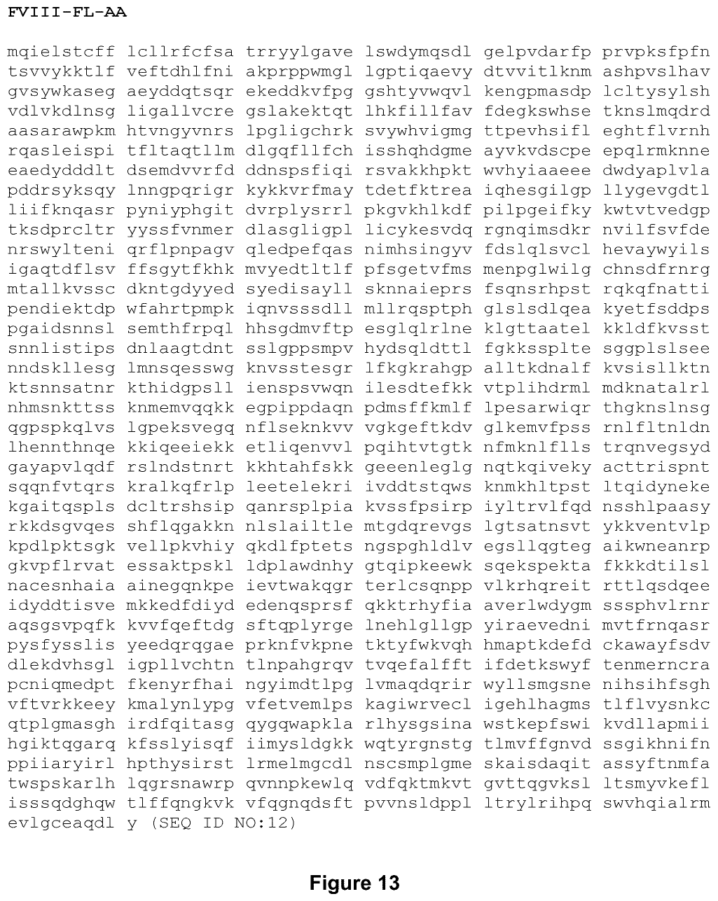

Unless otherwise specified herein, the numbering of Factor VIII amino acids refers to the corresponding amino acid in the full-length, wild-type human Factor VIII sequence (FVIII-FL-AA), presented as SEQ ID NO: 12 in FIG. 13. As such, when referring to an amino acid substitution in a Factor VIII variant protein disclosed herein, the recited amino acid number refers to the analogous (e.g., structurally or functionally equivalent) and/or homologous (e.g., evolutionarily conserved in the primary amino acid sequence) amino acid in the full-length, wild-type Factor VIII sequence. For example, a T2105N amino acid substitution refers to a T to N substitution at position 2105 of the full-length, wild-type human Factor VIII sequence (FVIII-FL-AA; SEQ ID NO: 12) and a T to N substitution at position 1211 of the Factor VIII variant protein encoded by CS04 (CS04-FL-AA; SEQ ID NO: 2).

As described herein, the Factor VIII amino acid numbering system is dependent on whether the Factor VIII signal peptide (e.g., amino acids 1-19 of the full-length, wild-type human Factor VIII sequence) is included. Where the signal peptide is included, the numbering is referred to as “signal peptide inclusive” or “SPI”. Where the signal peptide is not included, the numbering is referred to as “signal peptide exclusive” or “SPE.” For example, F328S is SPI numbering for the same amino acid as F309S, in SPE numbering. Unless otherwise indicated, all amino acid numbering refers to the corresponding amino acid in the full-length, wild-type human Factor VIII sequence (FVIII-FL-AA), presented as SEQ ID NO: 12 in FIG. 13.

As described herein, the codon-altered polynucleotides provide increased expression of transgenic Factor VIII in vivo (e.g., when administered as part of a gene therapy vector), as compared to the level of Factor VIII expression provided by a natively-coded Factor VIII construct (e.g., a polynucleotide encoding the same Factor VIII construct using the wild-type human codons). As used herein, the term “increased expression” refers to an increased level of transgenic Factor VIII activity in the blood of an animal administered the codon-altered polynucleotide encoding Factor VIII, as compared to the level of transgenic Factor VIII activity in the blood of an animal administered a natively-coded Factor VIII construct. The activity levels can be measured using any Factor VIII activity known in the art. An exemplary assay for determining Factor VIII activity is the Technochrome FVIII assay (Technoclone, Vienna, Austria).

In some embodiments, increased expression refers to at least 25% greater transgenic Factor VIII activity in the blood of an animal administered the codon-altered Factor VIII polynucleotide, as compared to the level of transgenic Factor VIII activity in the blood of an animal administered a natively coded Factor VIII polynucleotide. In some embodiments, increased expression refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, at least 20-fold greater, at least 25-fold greater, at least 30-fold greater, at least 40-fold greater, at least 50-fold greater, at least 60-fold greater, at least 70-fold greater, at least 80-fold greater, at least 90-fold greater, at least 100-fold greater, at least 125-fold greater, at least 150-fold greater, at least 175-fold greater, at least 200-fold greater, at least 225-fold greater, or at least 250-fold greater transgenic Factor VIII activity in the blood of an animal administered the codon-altered Factor VIII polynucleotide, as compared to the level of transgenic Factor VIII activity in the blood of an animal administered a natively coded Factor VIII polynucleotide.

As described herein, the codon-altered polynucleotides provide increased vector production, as compared to the level of vector production provided by a natively-coded Factor VIII construct (e.g., a polynucleotide encoding the same Factor VIII construct using the wild-type human codons). As used herein, the term “increased virus production” refers to an increased vector yield in cell culture (e.g., titer per liter culture) inoculated with the codon-altered polynucleotide encoding Factor VIII, as compared to the vector yield in cell culture inoculated with a natively-coded Factor VIII construct. The vector yields can be measured using any vector titer assay known in the art. An exemplary assay for determining vector yield (e.g., of an AAV vector) is qPCR targeting the AAV2 inverted terminal repeats (Aurnhammer, Human Gene Therapy Methods: Part B 23:18-28 (2012)).

In some embodiments, increased virus production refers to at least 25% greater codon-altered vector yield, as compared to the yield of a natively-coded Factor VIII construct in the same type of culture. In some embodiments, increased vector production refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, or at least 20-fold greater codon-altered vector yield, as compared to the yield of a natively-coded Factor VIII construct in the same type of culture.

As used herein, the term “hemophilia” refers to a group of disease states broadly characterized by reduced blood clotting or coagulation. Hemophilia may refer to Type A, Type B, or Type C hemophilia, or to the composite of all three diseases types. Type A hemophilia (hemophilia A) is caused by a reduction or loss of factor VIII (FVIII) activity and is the most prominent of the hemophilia subtypes. Type B hemophilia (hemophilia B) results from the loss or reduction of factor IX (FIX) clotting function. Type C hemophilia (hemophilia C) is a consequence of the loss or reduction in factor XI (FXI) clotting activity. Hemophilia A and B are X-linked diseases, while hemophilia C is autosomal. Conventional treatments for hemophilia include both prophylactic and on-demand administration of clotting factors, such as FVIII, FIX, including Bebulin®-VH, and FXI, as well as FEIBA-VH, desmopressin, and plasma infusions.

As used herein, the term “FVIII gene therapy” includes any therapeutic approach of providing a nucleic acid encoding Factor VIII to a patient to relieve, diminish, or prevent the reoccurrence of one or more symptoms (e.g., clinical factors) associated with hemophilia. The term encompasses administering any compound, drug, procedure, or regimen comprising a nucleic acid encoding a Factor VIII molecule, including any modified form of Factor VIII (e.g., Factor VIII variant), for maintaining or improving the health of an individual with hemophilia. One skilled in the art will appreciate that either the course of FVIII therapy or the dose of a FVIII therapeutic agent can be changed, e.g., based upon the results obtained in accordance with the present disclosure.

As used herein, the term “bypass therapy” includes any therapeutic approach of providing non-Factor VIII hemostatic agents, compounds or coagulation factors to a patient to relieve, diminish, or prevent the reoccurrence of one or more symptoms (e.g., clinical factors) associated with hemophilia. Non-Factor VIII compounds and coagulation factors include, but are not limited to, Factor VIII Inhibitor Bypass Activity (FEIBA), recombinant activated factor VII (FVIIa), prothrombin complex concentrates, and activated prothrombin complex concentrates. These non-Factor VIII compounds and coagulation factors may be recombinant or plasma-derived. One skilled in the art will appreciate that either the course of bypass therapy or the dose of bypass therapy can be changed, e.g., based upon the results obtained in accordance with the present disclosure.

As used herein, a “combination therapy” including administration of a nucleic acid encoding a Factor VIII molecule and a conventional hemophilia A therapeutic agent includes any therapeutic approach of providing both a nucleic acid encoding a Factor VIII molecule and a Factor VIII molecule and/or non-Factor VIII hemostatic agent (e.g., bypass therapeutic agent) to a patient to relieve, diminish, or prevent the reoccurrence of one or more symptoms (e.g., clinical factors) associated with hemophilia. The term encompasses administering any compound, drug, procedure, or regimen including a nucleic acid encoding a Factor VIII molecule, including any modified form of factor VIII, which is useful for maintaining or improving the health of an individual with hemophilia and includes any of the therapeutic agents described herein.

The terms “therapeutically effective amount or dose” or “therapeutically sufficient amount or dose” or “effective or sufficient amount or dose” refer to a dose that produces therapeutic effects for which it is administered. For example, a therapeutically effective amount of a drug useful for treating hemophilia can be the amount that is capable of preventing or relieving one or more symptoms associated with hemophilia. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “gene” refers to the segment of a DNA molecule that codes for a polypeptide chain (e.g., the coding region). In some embodiments, a gene is positioned by regions immediately preceding, following, and/or intervening the coding region that are involved in producing the polypeptide chain (e.g., regulatory elements such as a promoter, enhancer, polyadenylation sequence, 5′-untranslated region, 3′-untranslated region, or intron).

As used herein, the term “regulatory elements” refers to nucleotide sequences, such as promoters, enhancers, terminators, polyadenylation sequences, introns, etc, that provide for the expression of a coding sequence in a cell.

As used herein, the term “promoter element” refers to a nucleotide sequence that assists with controlling expression of a coding sequence. Generally, promoter elements are located 5′ of the translation start site of a gene. However, in certain embodiments, a promoter element may be located within an intron sequence, or 3′ of the coding sequence. In some embodiments, a promoter useful for a gene therapy vector is derived from the native gene of the target protein (e.g., a Factor VIII promoter). In some embodiments, a promoter useful for a gene therapy vector is specific for expression in a particular cell or tissue of the target organism (e.g., a liver-specific promoter). In yet other embodiments, one of a plurality of well characterized promoter elements is used in a gene therapy vector described herein. Non-limiting examples of well-characterized promoter elements include the CMV early promoter, the B-actin promoter, and the methyl CpG binding protein 2 (MeCP2) promoter. In some embodiments, the promoter is a constitutive promoter, which drives substantially constant expression of the target protein. In other embodiments, the promoter is an inducible promoter, which drives expression of the target protein in response to a particular stimulus (e.g., exposure to a particular treatment or agent). For a review of designing promoters for AAV-mediated gene therapy, see Gray et al. (Human Gene Therapy 22:1143-53 (2011)), the contents of which are expressly incorporated by reference in their entirety for all purposes.

As used herein, the term “vector” refers to any vehicle used to transfer a nucleic acid (e.g., encoding a Factor VIII gene therapy construct) into a host cell. In some embodiments, a vector includes a replicon, which functions to replicate the vehicle, along with the target nucleic acid. Non-limiting examples of vectors useful for gene therapy include plasmids, phages, cosmids, artificial chromosomes, and viruses, which function as autonomous units of replication in vivo. In some embodiments, a vector is a viral vehicle for introducing a target nucleic acid (e.g., a codon-altered polynucleotide encoding a Factor VIII variant). Many modified eukaryotic viruses useful for gene therapy are known in the art. For example, adeno-associated viruses (AAVs) are particularly well suited for use in human gene therapy because humans are a natural host for the virus, the native viruses are not known to contribute to any diseases, and the viruses illicit a mild immune response.

As used herein, the term “CpG island” refers to a region within a polynucleotide having a statistically elevated density of CpG dinucleotides. As used herein, a region of a polynucleotide (e.g., a polynucleotide encoding a codon-altered Factor VIII protein) is a CpG island if, over a 200-base pair window: (i) the region has GC content of greater than 50%, and (ii) the ratio of observed CpG dinucleotides per expected CpG dinucleotides is at least 0.6, as defined by the relationship:

$$\frac{N [ CpG ] ⋆ N [ length of window ]}{N [ C ] ⋆ N [ G ]} \geq 0.6 .$$

For additional information on methods for identifying CpG islands, see Gardiner-Garden M. et al., J Mol Biol., 196 (2): 261-82 (1987), the content of which is expressly incorporated herein by reference, in its entirety, for all purposes.

As used herein, the term “nucleic acid” refers to deoxyribonucleotides or ribonucleotides and polymers thereof in either single-or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

The term “amino acid” refers to naturally occurring and non-natural amino acids, including amino acid analogs and amino acid mimetics that function in a manner similar to the naturally occurring amino acids. Naturally occurring amino acids include those encoded by the genetic code, as well as those amino acids that are later modified, e.g., hydroxyproline, y-carboxyglutamate, and O-phosphoserine. Naturally occurring amino acids can include, e.g., D- and L-amino acids. The amino acids used herein can also include non-natural amino acids. Amino acid analogs refer to compounds that have the same basic chemical structure as a naturally occurring amino acid, i.e., any carbon that is bound to a hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of ordinary skill in the art will recognize that individual substitutions, deletions or additions to a nucleic acid or peptide sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

Conservative amino acid substitutions providing functionally similar amino acids are well known in the art. Dependent on the functionality of the particular amino acid, e.g., catalytic, structural, or sterically important amino acids, different groupings of amino acid may be considered conservative substitutions for each other. Table 1 provides groupings of amino acids that are considered conservative substitutions based on the charge and polarity of the amino acid, the hydrophobicity of the amino acid, the surface exposure/structural nature of the amino acid, and the secondary structure propensity of the amino acid.

TABLE 1
Groupings of conservative amino acid substitutions based on
the functionality of the residue in the protein.

| Important Feature | Conservative Groupings |
|---|---|
| Charge/Polarity | 1. H, R, and K |
| | 2. D and E |
| | 3. C, T, S, G, N, Q, and Y |
| | 4. A, P, M, L, I, V, F, and W |
| Hydrophobicity | 1. D, E, N, Q, R, and K |
| | 2. C, S, T, P, G, H, and Y |
| | 3. A, M, I, L, V, F, and W |
| Structural/Surface Exposure | 1. D, E, N, Q, H, R, and K |
| | 2. C, S, T, P, A, G, W, and Y |
| | 3. M, I, L, V, and F |
| Secondary Structure Propensity | 1. A, E, Q, H, K, M, L, and R |
| | 2. C, T, I, V, F, Y, and W |
| | 3. S, G, P, D, and N |
| Evolutionary Conservation | 1. D and E |
| | 2. H, K, and R |
| | 3. N and Q |
| | 4. S and T |
| | 5. L, I, and V |
| | 6. F, Y, and W |
| | 7. A and G |
| | 8. M and C |

The terms “identical” or percent “identity,” in the context of two or more nucleic acids or peptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using, e.g., a BLAST or BLAST 2.0 sequence comparison algorithms with default parameters described below, or by manual alignment and visual inspection.

As is known in the art, a number of different programs may be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc, all of which are incorporated by reference.

An example of a useful algorithm is PILEUP. PILEUP creates a multiple sequence alignment from a group of related sequences using progressive, pair wise alignments. It may also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989), both incorporated by reference. Useful PILEUP parameters including a default gap weight of 3.00, a default gap length weight of 0.10, and weighted end gaps.

Another example of a useful algorithm is the BLAST algorithm, described in: Altschul et al., J. Mol. Biol. 215, 403-410, (1990); Altschul et al., Nucleic Acids Res. 25:3389-3402 (1997); and Karlin et al., Proc. Natl. Acad. Sci. U.S.A. 90:5873-5787 (1993), both incorporated by reference. A particularly useful BLAST program is the WU-BLAST-2 program which was obtained from Altschul et al., Methods in Enzymology, 266:460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST, as reported by Altschul et al., Nucl. Acids Res., 25:3389-3402, incorporated by reference. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, “percent (%) nucleic acid sequence identity” with respect to the coding sequence of the polypeptides identified is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

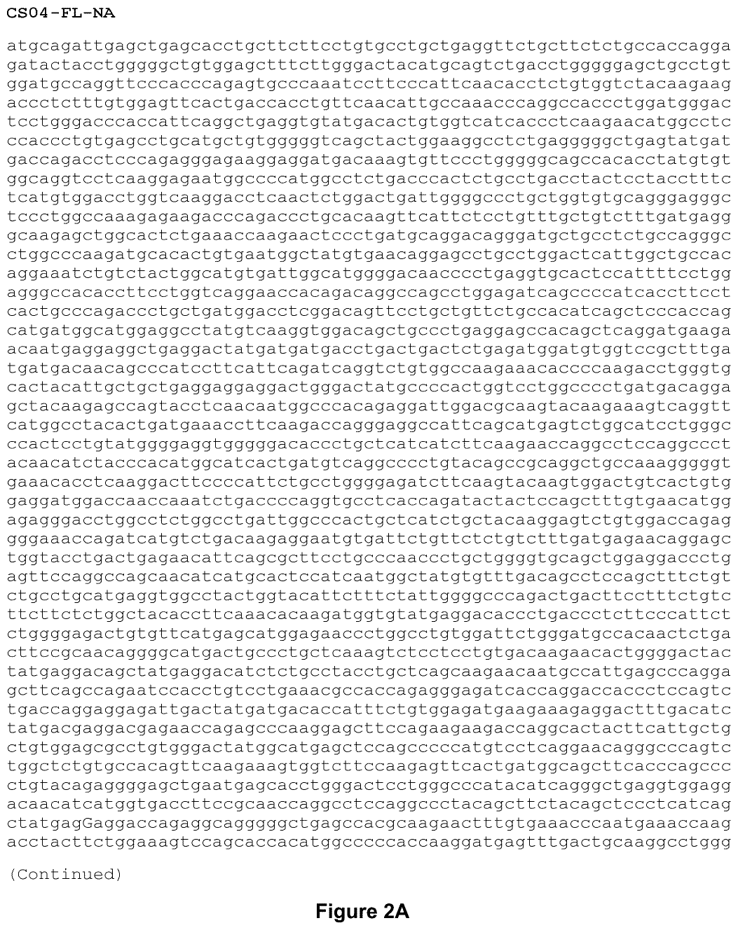

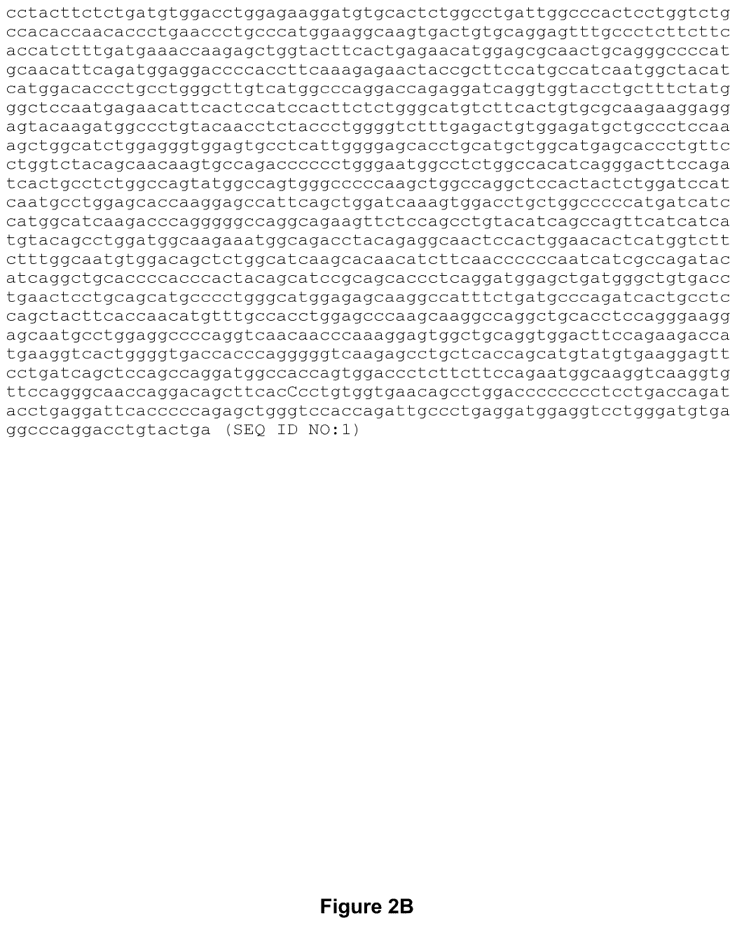

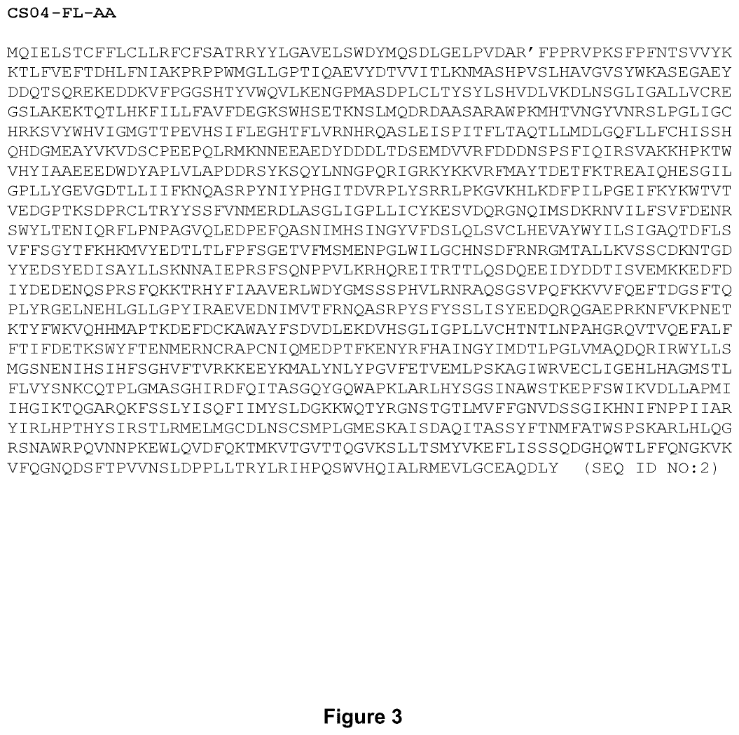

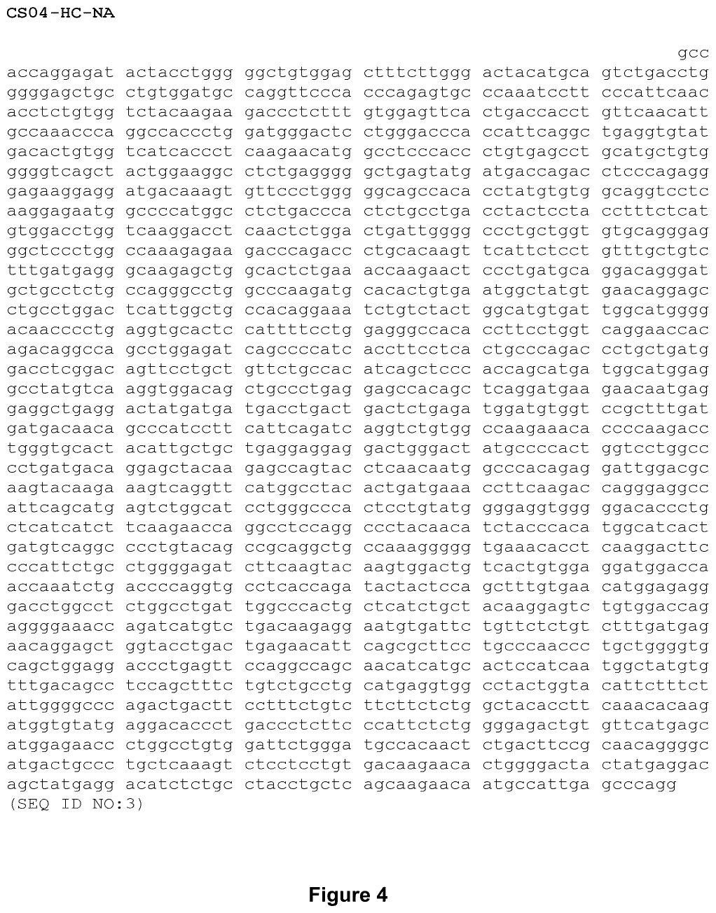

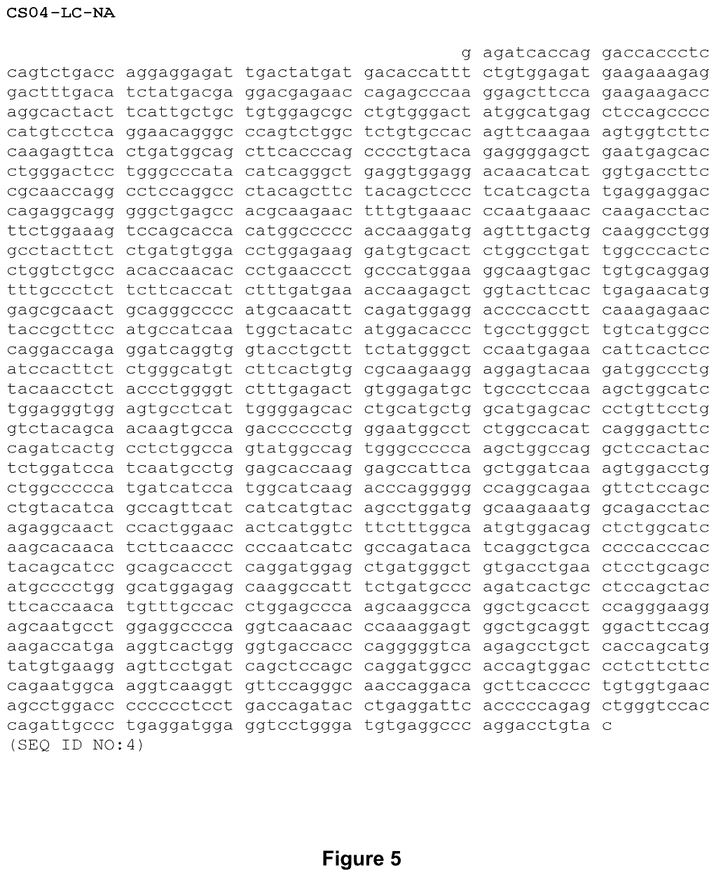

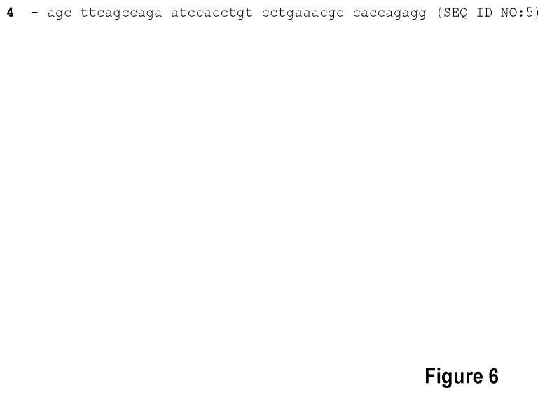

The alignment may include the introduction of gaps in the sequences to be aligned. In addition, for sequences which contain either more or fewer amino acids than the protein encoded by the sequence of FIG. 2 (SEQ ID NO: 1), it is understood that in one embodiment, the percentage of sequence identity will be determined based on the number of identical amino acids or nucleotides in relation to the total number of amino acids or nucleotides. Thus, for example, sequence identity of sequences shorter than that shown in FIG. 2 (SEQ ID NO: 1), as discussed below, will be determined using the number of nucleotides in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity may be calculated, for example, by dividing the number of matching identical residues by the total number of residues of the “shorter” sequence in the aligned region and multiplying by 100. The “longer” sequence is the one having the most actual residues in the aligned region.

The term “allelic variants” refers to polymorphic forms of a gene at a particular genetic locus, as well as cDNAs derived from mRNA transcripts of the genes, and the polypeptides encoded by them. The term “preferred mammalian codon” refers a subset of codons from among the set of codons encoding an amino acid that are most frequently used in proteins expressed in mammalian cells as chosen from the following list: Gly (GGC, GGG); Glu (GAG); Asp (GAC); Val (GTG, GTC); Ala (GCC, GCT); Ser (AGC, TCC); Lys (AAG); Asn (AAC); Met (ATG); Ile (ATC); Thr (ACC); Trp (TGG); Cys (TGC); Tyr (TAT, TAC); Leu (CTG); Phe (TTC); Arg (CGC, AGG, AGA); Gln (CAG); His (CAC); and Pro (CCC).

As used herein, the term codon-altered refers to a polynucleotide sequence encoding a polypeptide (e.g., a Factor VIII variant protein), where at least one codon of the native polynucleotide encoding the polypeptide has been changed to improve a property of the polynucleotide sequence. In some embodiments, the improved property promotes increased transcription of mRNA coding for the polypeptide, increased stability of the mRNA (e.g., improved mRNA half-life), increased translation of the polypeptide, and/or increased packaging of the polynucleotide within the vector. Non-limiting examples of alterations that can be used to achieve the improved properties include changing the usage and/or distribution of codons for particular amino acids, adjusting global and/or local GC content, removing AT-rich sequences, removing repeated sequence elements, adjusting global and/or local CpG dinucleotide content, removing cryptic regulatory elements (e.g., TATA box and CCAAT box elements), removing of intron/exon splice sites, improving regulatory sequences (e.g., introduction of a Kozak consensus sequence), and removing sequence elements capable of forming secondary structure (e.g., stem-loops) in the transcribed mRNA.

As discussed herein, there are various nomenclatures to refer to components of the disclosure herein. “CS-number” (e.g. “CS04”) refer to codon altered polynucleotides encoding FVIII polypeptides and/or the encoded polypeptides, including variants. For example, CS04-FL refers to the Full Length codon altered CS04 polynucleotide sequence or amino acid sequence (sometimes referred to herein as “CS04-FL-AA” for the Amino Acid sequence and “CS04-FL-NA” for the Nucleic Acid sequence) encoded by the CS04 polynucleotide sequence. Similarly, “CS04-LC” refers to either the codon altered nucleic acid sequence (“CS04-LC-NA”) encoding the light chain of a FVIII polypeptide or the amino acid sequence (also sometimes referred to herein as “CS04-LC-AA”) of the FVIII light chain encoded by the CS04 polynucleotide sequence. Likewise, CS04-HC, CS04-HC-AA and CS04-HC-NA are the same for the FVIII heavy chain. As will be appreciated by those in the art, for constructs such as CS04, that are only codon-altered (e.g. they do not contain additional amino acid substitutions as compared to Refacto), the amino acid sequences will be identical, as the amino acid sequences are not altered by the codon optimization. Thus, sequence constructs of the disclosure include, but are not limited to, CS04-FL-NA, CS04-FL-AA, CS04-LC-NA, CS04-LC-AA, CS04-HC-AA, and CS04-HC-NA.

### III. CODON-ALTERED FACTOR VIII VARIANTS

In some embodiments, the present disclosure provides codon-altered polynucleotides encoding Factor VIII variants. These codon-altered polynucleotides provide markedly improved expression of Factor VIII when administered in an AAV-based gene therapy construct. The codon-altered polynucleotides also demonstrate improved AAV-virion packaging, as compared to conventionally codon-optimized constructs. As demonstrated in Example 2 and Table 4, Applicants have achieve these advantages through the discovery of a codon-altered polynucleotide (CS04-FL-NA) encoding a Factor VIII polypeptide with human wild-type Factor VIII heavy and light chains, and a short, 14 amino acid, B-domain substituted linker (the “SQ” linker) containing a furin cleavage site to facilitate maturation of an active FVIIIa protein in vivo.

In one embodiment, a codon-altered polynucleotide provided herein has nucleotide sequences with high sequence identity to at least the sequences within CS04 (SEQ ID NO: 1) encoding the Factor VIII heavy chain and Factor VIII light chains. As known in the art, the B-domain of Factor VIII is dispensable for activity in vivo. Thus, in some embodiments, the codon-altered polynucleotides provided herein completely lack a Factor VIII B-domain. In some embodiments, the native Factor VIII B-domain is replaced with a short amino acid linker containing a furin cleavage site, e.g., the “SQ” linker consisting of amino acids 760-773 of the CS04 (SEQ ID NO: 5) constructs. The “SQ” linker is also referred to as BDLO04, (-AA for the amino acid sequence and -NA for the nucleotide sequence).

In one embodiment, the Factor VIII heavy and light chains encoded by the codon-altered polynucleotide are human Factor VIII heavy and light chains, respectively. In other embodiments, the Factor VIII heavy and light chains encoded by the codon-altered polynucleotide are heavy and light chain sequences from another mammal (e.g., porcine Factor VIII). In yet other embodiments, the Factor VIII heavy and light chains are chimeric heavy and light chains (e.g., a combination of human and a second mammalian sequence). In yet other embodiments, the Factor VIII heavy and light chains are humanized version of the heavy and light chains from another mammal, e.g., heavy and light chain sequences from another mammal in which human residues are substituted at select positions to reduce the immunogenicity of the resulting peptide when administered to a human.

The GC content of human genes varies widely, from less than 25% to greater than 90%. However, in general, human genes with higher GC contents are expressed at higher levels. For example, Kudla et al. (PLOS Biol., 4(6):80 (2006)) demonstrate that increasing a gene's GC content increases expression of the encoded polypeptide, primarily by increasing transcription and effecting a higher steady state level of the mRNA transcript. Generally, the desired GC content of a codon-optimized gene construct is equal or greater than 60%. However, native AAV genomes have GC contents of around 56%.

Accordingly, in some embodiments, the codon-altered polynucleotides provided herein have a CG content that more closely matches the GC content of native AAV virions (e.g., around 56% GC), which is lower than the preferred CG contents of polynucleotides that are conventionally codon-optimized for expression in mammalian cells (e.g., at or above 60% GC). As outlined in Example 1, CS04-FL-NA (SEQ ID NO: 1), which has a GC content of about 56%, has improved virion packaging as compared to similarly codon-altered coding sequences with higher GC content.

Thus, in some embodiments, the overall GC content of a codon-altered polynucleotide encoding a Factor VIII polypeptide is less than 60%. In some embodiments, the overall GC content of a codon-altered polynucleotide encoding a Factor VIII polypeptide is less than 59%. In some embodiments, the overall GC content of a codon-altered polynucleotide encoding a Factor VIII polypeptide is less than 58%. In some embodiments, the overall GC content of a codon-altered polynucleotide encoding a Factor VIII polypeptide is less than 57%. In some embodiments, the overall GC content of a codon-altered polynucleotide encoding a Factor VIII polypeptide is no more than 56%.

In some embodiments, the overall GC content of a codon-altered polynucleotide encoding a Factor VIII polypeptide is from 54% to 59%. In some embodiments, the overall GC content of a codon-altered polynucleotide encoding a Factor VIII polypeptide is from 55% to 59%. In some embodiments, the overall GC content of a codon-altered polynucleotide encoding a Factor VIII polypeptide is from 56% to 59%. In some embodiments, the overall GC content of a codon-altered polynucleotide encoding a Factor VIII polypeptide is from 54% to 58%. In some embodiments, the overall GC content of a codon-altered polynucleotide encoding a Factor VIII polypeptide is from 55% to 58%. In some embodiments, the overall GC content of a codon-altered polynucleotide encoding a Factor VIII polypeptide is from 56% to 58%. In some embodiments, the overall GC content of a codon-altered polynucleotide encoding a Factor VIII polypeptide is from 54% to 57%. In some embodiments, the overall GC content of a codon-altered polynucleotide encoding a Factor VIII polypeptide is from 55% to 57%. In some embodiments, the overall GC content of a codon-altered polynucleotide encoding a Factor VIII polypeptide is from 56% to 57%. In some embodiments, the overall GC content of a codon-altered polynucleotide encoding a Factor VIII polypeptide is from 54% to 56%. In some embodiments, the overall GC content of a codon-altered polynucleotide encoding a Factor VIII polypeptide is from 55% to 56%.

In some embodiments, the overall GC content of a codon-altered polynucleotide encoding a Factor VIII polypeptide is 56±0.5%. In some embodiments, the overall GC content of a codon-altered polynucleotide encoding a Factor VIII polypeptide is 56+0.4%. In some embodiments, the overall GC content of a codon-altered polynucleotide encoding a Factor VIII polypeptide is 56±0.3%. In some embodiments, the overall GC content of a codon-altered polynucleotide encoding a Factor VIII polypeptide is 56±0.2%. In some embodiments, the overall GC content of a codon-altered polynucleotide encoding a Factor VIII polypeptide is 56±0.1%. In some embodiments, the overall GC content of a codon-altered polynucleotide encoding a Factor VIII polypeptide is 56%.

### A. Factor VIII B-Domain Substituted Linkers

In some embodiments, the linkage between the FVIII heavy chain and the light chain (e.g., the B-domain in wild-type Factor VIII) is further altered. Due to size constraints of AAV packaging capacity, B-domain deleted, truncated, and or linker substituted variants should improve the efficacy of the FVIII gene therapy construct. The most conventionally used B-domain substituted linker is that of SQ FVIII, which retains only 14 amino acids of the B domain as linker sequence. Another variant of porcine VIII (“OBI-1,” described in U.S. Pat. No. 6,458,563) is well expressed in CHO cells, and has a slightly longer linker of 24 amino acids. In some embodiments, the Factor VIII constructs encoded by the codon-altered polynucleotides described herein include an SQ-type B-domain linker sequence. In other embodiments, the Factor VIII constructs encoded by the codon-altered polynucleotides described herein include an OBI-1-type B-domain linker sequence.

In some embodiments, the encoded Factor VIII polypeptides described herein include an SQ-type B-domain linker (SFSQNPPVLKRHQR; BDL-SQ-AA; SEQ ID NO: 13), including amino acids 760-762/1657-1667 of the wild-type human Factor VIII B-domain (FVIII-FL-AA; SEQ ID NO: 12) (Sandberg et al. Thromb. Haemost. 85:93 (2001)). In some embodiments, the SQ-type B-domain linker has one amino acid substitution relative to the corresponding wild-type sequence. In some embodiments, the SQ-type B-domain linker has two amino acid substitutions relative to the corresponding wild-type sequence.

In some embodiments, the encoded Factor VIII polypeptides described herein include a Greengene-type B-domain linker, including amino acids 760/1582-1667 of the wild-type human Factor VIII B-domain (FVIII-FL-AA; SEQ ID NO: 12) (Oh et al., Biotechnol. Prog., 17:1999 (2001)). In some embodiments, the Greengene-type B-domain linker has one amino acid substitution relative to the corresponding wild-type sequence. In some embodiments, the Greengene-type B-domain linker has two amino acid substitutions relative to the corresponding wild-type sequence.

In some embodiments, the encoded Factor VIII polypeptides described herein include an extended SQ-type B-domain linker, including amino acids 760-769/1657-1667 of the wild-type human Factor VIII B-domain (FVIII-FL-AA; SEQ ID NO: 12) (Thim et al., Haemophilia, 16:349 (2010)). In some embodiments, the extended SQ-type B-domain linker has one amino acid substitution relative to the corresponding wild-type sequence. In some embodiments, the extended SQ-type B-domain linker has two amino acid substitutions relative to the corresponding wild-type sequence.

In some embodiments, the encoded Factor VIII polypeptides described herein include a porcine OBI-1-type B-domain linker, including ir the amino acids SFAQNSRPPSASAPKPPVLRRHQR (SEQ ID NO: 14) from the wild-type porcine Factor VIII B-domain (Toschi et al., Curr. Opin. Mol. Ther. 12:517 (2010)). In some embodiments, the porcine OBI-1-type B-domain linker has one amino acid substitution relative to the corresponding wild-type sequence. In some embodiments, the porcine OBI-1-type B-domain linker has two amino acid substitutions relative to the corresponding wild-type sequence.

In some embodiments, the encoded Factor VIII polypeptides described herein include a human OBI-1-type B-domain linker, including amino acids 760-772/1655-1667 of the wild-type human Factor VIII B-domain (FVIII-FL-AA; SEQ ID NO: 12). In some embodiments, the human OBI-1-type B-domain linker has one amino acid substitution relative to the corresponding wild-type sequence. In some embodiments, the human OBI-1-type B-domain linker has two amino acid substitutions relative to the corresponding wild-type sequence.

In some embodiments, the encoded Factor VIII polypeptides described herein include an 08-type B-domain linker, including the amino acids SFSQNSRHQAYRYRRG (SEQ ID NO: 15) from the wild-type porcine Factor VIII B-domain (Toschi et al., Curr. Opin. Mol. Ther. 12:517 (2010)). In some embodiments, the porcine OBI-1-type B-domain linker has one amino acid substitution relative to the corresponding wild-type sequence. In some embodiments, the porcine OBI-1-type B-domain linker has two amino acid substitutions relative to the corresponding wild-type sequence.

### B. Codon-Altered Polynucleotides Encoding a Factor VIII Variant with a Cleavable Linker

### CS04 Codon Altered Polynucleotides

In one embodiment, the codon-altered polynucleotides provided herein include a nucleotide sequence encoding a Factor VIII variant polypeptide with a linker that is cleavable in vivo. The Factor VIII polypeptide includes a Factor VIII light chain, a Factor VIII heavy chain, and a polypeptide linker joining the C-terminus of the heavy chain to the N-terminus of the light chain. The heavy chain of the Factor VIII polypeptide is encoded by a first nucleotide sequence having high sequence identity to CS04-HC-NA (SEQ ID NO: 3), which is the portion of CS04-FL-NA (SEQ ID NO: 1) encoding for a Factor VIII heavy chain. The light chain of the Factor VIII polypeptide is encoded by a second nucleotide sequence with high sequence identity to CS04-LC-NA (SEQ ID NO: 4), which is the portion of CS04-FL-NA (SEQ ID NO: 1) encoding for a Factor VIII light chain. The polypeptide linker includes a furin cleavage site, which allows for maturation in vivo (e.g., after expression in vivo or administration of the precursor polypeptide).

In some embodiments, the first and second nucleotide sequences have at least 95% sequence identity to CS04-HC-NA and CS04-LC-NA (SEQ ID NOS 3 and 4), respectively. In some embodiments, the first and second nucleotide sequences have at least 96% sequence identity to CS04-HC-NA and CS04-LC-NA (SEQ ID NOS 3 and 4), respectively. In some embodiments, the first and second nucleotide sequences have at least 97% sequence identity to CS04-HC-NA and CS04-LC-NA (SEQ ID NOS 3 and 4), respectively. In some embodiments, the first and second nucleotide sequences have at least 98% sequence identity to CS04-HC-NA and CS04-LC-NA (SEQ ID NOS 3 and 4), respectively. In some embodiments, the first and second nucleotide sequences have at least 99% sequence identity to CS04-HC-NA and CS04-LC-NA (SEQ ID NOS 3 and 4), respectively. In some embodiments, the first and second nucleotide sequences have at least 99.5% sequence identity to CS04-HC-NA and CS04-LC-NA (SEQ ID NOS 3 and 4), respectively. In some embodiments, the first and second nucleotide sequences have at least 99.9% sequence identity to CS04-HC-NA and CS04-LC-NA (SEQ ID NOS 3 and 4), respectively. In some embodiments, the first and second nucleotide sequences are identical to CS04-HC-NA and CS04-LC-NA (SEQ ID NOS 3 and 4), respectively.

In some embodiments, the polypeptide linker of the Factor VIII construct is encoded by a third nucleotide sequence having high sequence identity to BDLO04 (SEQ ID NO: 5), which encodes the 14-amino acid linker corresponding to amino acids 760-773 of CS04-FL-AA (SEQ ID NO: 2). In some embodiments, the third nucleotide sequence has at least 95% identity to BDLO04 (SEQ ID NO: 5). In some embodiments, the third nucleotide sequence has at least 96% identity to BDLO04 (SEQ ID NO: 5). In some embodiments, the third nucleotide sequence has at least 97% identity to BDLO04 (SEQ ID NO: 5). In some embodiments, the third nucleotide sequence has at least 98% identity to BDLO04 (SEQ ID NO: 5). In some embodiments, the third nucleotide sequence is identical to BDLO04 (SEQ ID NO: 5).

In some embodiments, the codon-altered polynucleotide has a nucleotide sequence with high sequence identity to CS04-FL-NA (SEQ ID NO: 1). In some embodiments, the nucleotide sequence has at least 95% identity to CS04-FL-NA (SEQ ID NO: 1). In some embodiments, the nucleotide sequence has at least 96% identity to CS04-FL-NA (SEQ ID NO: 1). In some embodiments, the nucleotide sequence has at least 97% identity to CS04-FL-NA (SEQ ID NO: 1). In some embodiments, the nucleotide sequence has at least 98% identity to CS04-FL-NA (SEQ ID NO: 1). In some embodiments, the nucleotide sequence has at least 99% identity to CS04-FL-NA (SEQ ID NO: 1). In some embodiments, the nucleotide sequence has at least 99.5% identity to CS04-FL-NA (SEQ ID NO: 1). In some embodiments, the nucleotide sequence has at least 99.9% identity to CS04-FL-NA (SEQ ID NO: 1). In some embodiments, the nucleotide sequence is identical to CS04-FL-NA (SEQ ID NO: 1).

In some embodiments, the Factor VIII variant encoded by the codon-altered polynucleotide has an amino acid sequence with high sequence identity to CS04-FL-AA (SEQ ID NO: 2). In some embodiments, the amino acid sequence has at least 97% identity to CS04-FL-AA (SEQ ID NO: 2). In some embodiments, the amino acid sequence has at least 98% identity to CS04-FL-AA (SEQ ID NO: 2). In some embodiments, the amino acid sequence has at least 99% identity to CS04-FL-AA (SEQ ID NO: 2). In some embodiments, the amino acid sequence has at least 99.5% identity to CS04-FL-AA (SEQ ID NO: 2). In some embodiments, the amino acid sequence has at least 99.9% identity to CS04-FL-AA (SEQ ID NO: 2). In some embodiments, the amino acid sequence is identical to CS04-FL-AA (SEQ ID NO: 2).

### C. Factor VIII Expression Vectors

In some embodiments, the codon-altered polynucleotides described herein are integrated into expression vectors. Non-limiting examples of expression vectors include viral vectors (e.g., vectors suitable for gene therapy), plasmid vectors, bacteriophage vectors, cosmids, phagemids, artificial chromosomes, and the like.

Non-limiting examples of viral vectors include: retrovirus, e.g., Moloney murine leukemia virus (MMLV), Harvey murine sarcoma virus, murine mammary tumor virus, and Rous sarcoma virus; adenoviruses, adeno-associated viruses; SV40-type viruses; polyomaviruses; Epstein-Barr viruses; papilloma viruses; herpes viruses; vaccinia viruses; and polio viruses.

In some embodiments, the codon-altered polynucleotides described herein are integrated into a gene therapy vector. In some embodiments, the gene therapy vector is a retrovirus, and particularly a replication-deficient retrovirus. Protocols for the production of replication-deficient retroviruses are known in the art. For review, see Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

In one embodiment, the gene therapy vector is an adeno-associated virus (AAV) based gene therapy vector. AAV systems have been described previously and are generally well known in the art (Kelleher and Vos, Biotechniques, 17(6):1110-17 (1994); Cotten et al., Proc Natl Acad Sci USA, 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20 (4): 699-708 (2012), each incorporated herein by reference in their entireties for all purposes). Details concerning the generation and use of rAAV vectors are described, for example, in U.S. Pat. Nos. 5,139,941 and 4,797,368, each incorporated herein by reference in their entireties for all purposes. In a particular embodiment, the AAV vector is an AAV-8 vector.

In some embodiments, the codon-altered polynucleotides described herein are integrated into a retroviral expression vector. These systems have been described previously, and are generally well known in the art (Mann et al., Cell, 33:153-159, 1983; Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986). In a specific embodiment, the retroviral vector is a lentiviral vector (see, for example, Naldini et al., Science, 272(5259):263-267, 1996; Zufferey et al., Nat Biotechnol, 15(9):871-875, 1997; Blomer et al., J Virol., 71(9):6641-6649, 1997; U.S. Pat. Nos. 6,013,516 and 5,994,136).

A wide variety of vectors can be used for the expression of a Factor VIII polypeptide from a codon-altered polypeptide in cell culture, including eukaryotic and prokaryotic expression vectors. In certain embodiments, a plasmid vector is contemplated for use in expressing a Factor VIII polypeptide in cell culture. In general, plasmid vectors containing replicon and control sequences which are derived from species compatible with the host cell are used in connection with these hosts. The vector can carry a replication site, as well as marking sequences which are capable of providing phenotypic selection in transformed cells. The plasmid will include the codon-altered polynucleotide encoding the Factor VIII polypeptide, operably linked to one or more control sequences, for example, a promoter.

Non-limiting examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

### D. Dosing

The invention provides the administration of the codon-optimized constructs of the invention to human patients that have been diagnosed with hemophilia A (a “hemophilia A patient” or “patient”). In general, as outlined herein, the administration is done using AAV particles that contain the codon-optimized constructs of the invention. Furthermore, as is more fully described below, the administration of the constructs of the invention can be augmented by the administration of prednisolone or prednisone as well.

### 2×10¹² Adeno-Associated Virus (AAV) Particles Per Kilogram Body Weight

In one aspect, the disclosure provides a method for treating hemophilia A including intravenously infusing (e.g., by peripheral intravenous infusion), to a hemophilia A patient, a dose of 2×10¹² adeno-associated virus (AAV) particles per kilogram body weight of the human patient, where the AAV particles include a codon-altered polynucleotide encoding a Factor VIII polypeptide, having high sequence identity to SEQ ID NO: 1 (CS04-FL-NA).

In one embodiment, the codon-altered polynucleotide having high sequence identity to SEQ ID NO: 1 (CS04-FL-NA), that is administered to the human patient at a dose of 2×10¹² adeno-associated virus (AAV) particles per kilogram body weight of the human patient, encodes a Factor VIII variant polypeptide with a linker that is cleavable in vivo. The Factor VIII polypeptide includes a Factor VIII light chain, a Factor VIII heavy chain, and a polypeptide linker joining the C-terminus of the heavy chain to the N-terminus of the light chain. The heavy chain of the Factor VIII polypeptide is encoded by a first nucleotide sequence having high sequence identity to CS04-HC-NA (SEQ ID NO: 3), which is the portion of CS04-FL-NA (SEQ ID NO: 1) encoding for a Factor VIII heavy chain. The light chain of the Factor VIII polypeptide is encoded by a second nucleotide sequence with high sequence identity to CS04-LC-NA (SEQ ID NO: 4), which is the portion of CS04-FL-NA (SEQ ID NO: 1) encoding for a Factor VIII light chain. The polypeptide linker includes a furin cleavage site, which allows for maturation in vivo (e.g., after expression in vivo or administration of the precursor polypeptide).

In some embodiments, the first and second nucleotide sequences have at least 95% sequence identity to CS04-HC-NA and CS04-LC-NA (SEQ ID NOS 3 and 4), respectively. In some embodiments, the first and second nucleotide sequences have at least 96% sequence identity to CS04-HC-NA and CS04-LC-NA (SEQ ID NOS 3 and 4), respectively. In some embodiments, the first and second nucleotide sequences have at least 97% sequence identity to CS04-HC-NA and CS04-LC-NA (SEQ ID NOS 3 and 4), respectively. In some embodiments, the first and second nucleotide sequences have at least 98% sequence identity to CS04-HC-NA and CS04-LC-NA (SEQ ID NOS 3 and 4), respectively. In some embodiments, the first and second nucleotide sequences have at least 99% sequence identity to CS04-HC-NA and CS04-LC-NA (SEQ ID NOS 3 and 4), respectively. In some embodiments, the first and second nucleotide sequences have at least 99.5% sequence identity to CS04-HC-NA and CS04-LC-NA (SEQ ID NOS 3 and 4), respectively. In some embodiments, the first and second nucleotide sequences have at least 99.9% sequence identity to CS04-HC-NA and CS04-LC-NA (SEQ ID NOS 3 and 4), respectively. In some embodiments, the first and second nucleotide sequences are identical to CS04-HC-NA and CS04-LC-NA (SEQ ID NOS 3 and 4), respectively. In these embodiments, the amino acid sequence encoded by these nucleotide sequences are identical to CS04-HC-AA and CS04-LC-AA.

In some embodiments, the polypeptide linker of the Factor VIII construct is encoded by a third nucleotide sequence having high sequence identity to BDLO04 (SEQ ID NO: 5), which encodes the 14-amino acid linker corresponding to amino acids 760-773 of CS04-FL-AA (SEQ ID NO: 2). In some embodiments, the third nucleotide sequence has at least 95% identity to BDLO04 (SEQ ID NO: 5). In some embodiments, the third nucleotide sequence has at least 96% identity to BDLO04 (SEQ ID NO: 5). In some embodiments, the third nucleotide sequence has at least 97% identity to BDLO04 (SEQ ID NO: 5). In some embodiments, the third nucleotide sequence has at least 98% identity to BDLO04 (SEQ ID NO: 5). In some embodiments, the third nucleotide sequence is identical to BDLO04 (SEQ ID NO: 5). In these embodiments, the amino acid sequence encoded by these nucleotide sequences are identical to amino acids 760-773 of CS04-FL-AA (SEQ ID NO: 2). In some embodiments, the codon-altered polynucleotide), that is administered to the human patient at a dose of 2×10¹² adeno-associated virus (AAV) particles per kilogram body weight of the human patient, has a nucleotide sequence with high sequence identity to CS04-FL-NA (SEQ ID NO: 1). In some embodiments, the nucleotide sequence has at least 95% identity to CS04-FL-NA (SEQ ID NO: 1). In some embodiments, the nucleotide sequence has at least 96% identity to CS04-FL-NA (SEQ ID NO: 1). In some embodiments, the nucleotide sequence has at least 97% identity to CS04-FL-NA (SEQ ID NO: 1). In some embodiments, the nucleotide sequence has at least 98% identity to CS04-FL-NA (SEQ ID NO: 1). In some embodiments, the nucleotide sequence has at least 99% identity to CS04-FL-NA (SEQ ID NO: 1). In some embodiments, the nucleotide sequence has at least 99.5% identity to CS04-FL-NA (SEQ ID NO: 1). In some embodiments, the nucleotide sequence has at least 99.9% identity to CS04-FL-NA (SEQ ID NO: 1). In some embodiments, the nucleotide sequence is identical to CS04-FL-NA (SEQ ID NO: 1). In these embodiments, the amino acid sequence encoded by these nucleotide sequences is identical to CS04-FL-AA.

In some embodiments, the Factor VIII variant encoded by the codon-altered polynucleotide has an amino acid sequence with high sequence identity to CS04-FL-AA (SEQ ID NO: 2). In some embodiments, the amino acid sequence has at least 97% identity to CS04-FL-AA (SEQ ID NO: 2). In some embodiments, the amino acid sequence has at least 98% identity to CS04-FL-AA (SEQ ID NO: 2). In some embodiments, the amino acid sequence has at least 99% identity to CS04-FL-AA (SEQ ID NO: 2). In some embodiments, the amino acid sequence has at least 99.5% identity to CS04-FL-AA (SEQ ID NO: 2). In some embodiments, the amino acid sequence has at least 99.9% identity to CS04-FL-AA (SEQ ID NO: 2). In some embodiments, the amino acid sequence is identical to CS04-FL-AA (SEQ ID NO: 2).

Accordingly, in one embodiment, the disclosure provides a method for treating hemophilia A that includes intravenously infusing, to a hemophilia A patient, a dose of 2×10¹² adeno-associated virus (AAV) particles per kilogram body weight of the human patient, where the AAV particles include a polynucleotide having a nucleic acid sequence of SEQ ID NO: 1 (CS04-FL-NA).

In some embodiments, the AAV particles are administered in a single dose by intravenous infusion (e.g., into a vein in the patient's arm). In some embodiments, a portion of the single dose is administered, the patient is monitored for signs of an adverse reaction to the administration for a brief period of time (e.g., 30 minutes), and then (e.g., if no signs of an adverse reaction appear) the remaining portion of the single dose is administered to the patient.

In some embodiments, the human patient administered the AAV particles has severe hemophilia A. For example, in some embodiments, the patient has a level of Factor VIII activity in their blood stream, when not receiving Factor VIII replacement therapy, that is less than 2% of the amount of Factor VIII activity found in a reference blood sample, e.g., a blood sample with normal Factor VIII activity (e.g., a blood sample from a subject determined not to have hemophilia A), or an average Factor VIII activity found in the blood samples of a plurality of subjects determining not to have hemophilia A. In some embodiments, the subject has a level of Factor VIII activity in their blood stream, when not receiving Factor VIII replacement therapy, that is less than 2% of the amount of Factor VIII activity found in a reference blood sample.

In some embodiments, the human patient administered the AAV particles does not have inhibitors to FVIII (e.g., Factor VIII inhibitor antibodies), does not have haemostatic defects other than severe hemophilia A, does not have chronic hepatic dysfunction, and/or does not have severe renal impairment.

Accordingly, in some embodiments, the methods described herein include a step of qualifying a patient for administration of a dose of 2×10¹² adeno-associated virus (AAV) particles per kilogram body weight of the human patient, where the AAV particles include a codon-altered polynucleotide encoding a Factor VIII polypeptide, having high sequence identity to SEQ ID NO: 1 (CS04-FL-NA). The method includes determining a level of Factor VIII activity in the blood stream of the patient, when the patient is not receiving a Factor VIII replacement therapy, and qualifying the patient for administration of the AAV particles when the level of Factor VIII activity in the patient's blood stream is less than about 2%, or less than about 1%, of the level of Factor VIII in a reference samples. In some embodiments, the method includes determining whether the patient has one or more of inhibitors to FVIII (e.g., Factor VIII inhibitor antibodies), a haemostatic defect other than severe hemophilia A, chronic hepatic dysfunction, and severe renal impairment, and disqualifying the patient if they have any of the enumerated conditions.

### 6×10¹² Adeno-Associated Virus (AAV) Particles Per Kilogram Body Weight

In one aspect, the disclosure provides a method for treating hemophilia A including intravenously infusing (e.g., by peripheral intravenous infusion), to a hemophilia A patient, a dose of 6×10¹² adeno-associated virus (AAV) particles per kilogram body weight of the human patient, where the AAV particles include a codon-altered polynucleotide encoding a Factor VIII polypeptide, having high sequence identity to SEQ ID NO: 1 (CS04-FL-NA).

In one embodiment, the codon-altered polynucleotide having high sequence identity to SEQ ID NO: 1 (CS04-FL-NA), that is administered to the human patient at a dose of 6×10¹² adeno-associated virus (AAV) particles per kilogram body weight of the human patient, encodes a Factor VIII variant polypeptide with a linker that is cleavable in vivo. The Factor VIII polypeptide includes a Factor VIII light chain, a Factor VIII heavy chain, and a polypeptide linker joining the C-terminus of the heavy chain to the N-terminus of the light chain. The heavy chain of the Factor VIII polypeptide is encoded by a first nucleotide sequence having high sequence identity to CS04-HC-NA (SEQ ID NO: 3), which is the portion of CS04-FL-NA (SEQ ID NO: 1) encoding for a Factor VIII heavy chain. The light chain of the Factor VIII polypeptide is encoded by a second nucleotide sequence with high sequence identity to CS04-LC-NA (SEQ ID NO: 4), which is the portion of CS04-FL-NA (SEQ ID NO: 1) encoding for a Factor VIII light chain. The polypeptide linker includes a furin cleavage site, which allows for maturation in vivo (e.g., after expression in vivo or administration of the precursor polypeptide).

In some embodiments, the first and second nucleotide sequences have at least 95% sequence identity to CS04-HC-NA and CS04-LC-NA (SEQ ID NOS 3 and 4), respectively. In some embodiments, the first and second nucleotide sequences have at least 96% sequence identity to CS04-HC-NA and CS04-LC-NA (SEQ ID NOS 3 and 4), respectively. In some embodiments, the first and second nucleotide sequences have at least 97% sequence identity to CS04-HC-NA and CS04-LC-NA (SEQ ID NOS 3 and 4), respectively. In some embodiments, the first and second nucleotide sequences have at least 98% sequence identity to CS04-HC-NA and CS04-LC-NA (SEQ ID NOS 3 and 4), respectively. In some embodiments, the first and second nucleotide sequences have at least 99% sequence identity to CS04-HC-NA and CS04-LC-NA (SEQ ID NOS 3 and 4), respectively. In some embodiments, the first and second nucleotide sequences have at least 99.5% sequence identity to CS04-HC-NA and CS04-LC-NA (SEQ ID NOS 3 and 4), respectively. In some embodiments, the first and second nucleotide sequences have at least 99.9% sequence identity to CS04-HC-NA and CS04-LC-NA (SEQ ID NOS 3 and 4), respectively. In some embodiments, the first and second nucleotide sequences are identical to CS04-HC-NA and CS04-LC-NA (SEQ ID NOS 3 and 4), respectively. In these embodiments, the amino acid sequence encoded by these nucleotide sequences are identical to CS04-HC-AA and CS04-LC-AA.

In some embodiments, the polypeptide linker of the Factor VIII construct is encoded by a third nucleotide sequence having high sequence identity to BDLO04 (SEQ ID NO: 5), which encodes the 14-amino acid linker corresponding to amino acids 760-773 of CS04-FL-AA (SEQ ID NO: 2). In some embodiments, the third nucleotide sequence has at least 95% identity to BDLO04 (SEQ ID NO: 5). In some embodiments, the third nucleotide sequence has at least 96% identity to BDLO04 (SEQ ID NO: 5). In some embodiments, the third nucleotide sequence has at least 97% identity to BDLO04 (SEQ ID NO: 5). In some embodiments, the third nucleotide sequence has at least 98% identity to BDLO04 (SEQ ID NO: 5). In some embodiments, the third nucleotide sequence is identical to BDLO04 (SEQ ID NO: 5). In these embodiments, the amino acid sequence encoded by these nucleotide sequences are identical to amino acids 760-773 of CS04-FL-AA (SEQ ID NO: 2).

In some embodiments, the codon-altered polynucleotide), that is administered to the human patient at a dose of 6×10¹² adeno-associated virus (AAV) particles per kilogram body weight of the human patient, has a nucleotide sequence with high sequence identity to CS04-FL-NA (SEQ ID NO: 1). In some embodiments, the nucleotide sequence has at least 95% identity to CS04-FL-NA (SEQ ID NO: 1). In some embodiments, the nucleotide sequence has at least 96% identity to CS04-FL-NA (SEQ ID NO: 1). In some embodiments, the nucleotide sequence has at least 97% identity to CS04-FL-NA (SEQ ID NO: 1). In some embodiments, the nucleotide sequence has at least 98% identity to CS04-FL-NA (SEQ ID NO: 1). In some embodiments, the nucleotide sequence has at least 99% identity to CS04-FL-NA (SEQ ID NO: 1). In some embodiments, the nucleotide sequence has at least 99.5% identity to CS04-FL-NA (SEQ ID NO: 1). In some embodiments, the nucleotide sequence has at least 99.9% identity to CS04-FL-NA (SEQ ID NO: 1). In some embodiments, the nucleotide sequence is identical to CS04-FL-NA (SEQ ID NO: 1). In these embodiments, the amino acid sequence encoded by these nucleotide sequences is identical to CS04-FL-AA.

In some embodiments, the Factor VIII variant encoded by the codon-altered polynucleotide has an amino acid sequence with high sequence identity to CS04-FL-AA (SEQ ID NO: 2). In some embodiments, the amino acid sequence has at least 97% identity to CS04-FL-AA (SEQ ID NO: 2). In some embodiments, the amino acid sequence has at least 98% identity to CS04-FL-AA (SEQ ID NO: 2). In some embodiments, the amino acid sequence has at least 99% identity to CS04-FL-AA (SEQ ID NO: 2). In some embodiments, the amino acid sequence has at least 99.5% identity to CS04-FL-AA (SEQ ID NO: 2). In some embodiments, the amino acid sequence has at least 99.9% identity to CS04-FL-AA (SEQ ID NO: 2). In some embodiments, the amino acid sequence is identical to CS04-FL-AA (SEQ ID NO: 2).

Accordingly, in one embodiment, the disclosure provides a method for treating hemophilia A that includes intravenously infusing, to a hemophilia A patient, a dose of 6×10¹² adeno-associated virus (AAV) particles per kilogram body weight of the human patient, where the AAV particles include a polynucleotide having a nucleic acid sequence of SEQ ID NO: 1 (CS04-FL-NA).

In some embodiments, the AAV particles are administered in a single dose by intravenous infusion (e.g., into a vein in the patient's arm). In some embodiments, a portion of the single dose is administered, the patient is monitored for signs of an adverse reaction to the administration for a brief period of time (e.g., 30 minutes), and then (e.g., if no signs of an adverse reaction appear) the remaining portion of the single dose is administered to the patient.

In some embodiments, the human patient administered the AAV particles has severe hemophilia A. For example, in some embodiments, the patient has a level of Factor VIII activity in their blood stream, when not receiving Factor VIII replacement therapy, that is less than 2% of the amount of Factor VIII activity found in a reference blood sample, e.g., a blood sample with normal Factor VIII activity (e.g., a blood sample from a subject determined not to have hemophilia A), or an average Factor VIII activity found in the blood samples of a plurality of subjects determining not to have hemophilia A. In some embodiments, subject has a level of Factor VIII activity in their blood stream, when not receiving Factor VIII replacement therapy, that is less than 2% of the amount of Factor VIII activity found in a reference blood sample.

In some embodiments, the human patient administered the AAV particles does not have inhibitors to FVIII (e.g., Factor VIII inhibitor antibodies), does not have haemostatic defects other than severe hemophilia A, does not have chronic hepatic dysfunction, and/or does not have severe renal impairment.

Accordingly, in some embodiments, the methods described herein include a step of qualifying a patient for administration of a dose of 6×10¹² adeno-associated virus (AAV) particles per kilogram body weight of the human patient, where the AAV particles include a codon-altered polynucleotide encoding a Factor VIII polypeptide, having high sequence identity to SEQ ID NO: 1 (CS04-FL-NA). The method includes determining a level of Factor VIII activity in the blood stream of the patient, when the patient is not receiving a Factor VIII replacement therapy, and qualifying the patient for administration of the AAV particles when the level of Factor VIII activity in the patient's blood stream is less than about 2%, or less than about 1%, of the level of Factor VIII in a reference samples. In some embodiments, the method includes determining whether the patient has one or more of inhibitors to FVIII (e.g., Factor VIII inhibitor antibodies), a haemostatic defect other than severe hemophilia A, chronic hepatic dysfunction, and severe renal impairment, and disqualifying the patient if they have any of the enumerated conditions.

### Co-Administration with Prednisolone or Prednisone

In some embodiments, the methods described above for treating hemophilia A by administering AAV particles at either dose also include administering, to the human patient, a course of prednisolone or prednisone, e.g., to reduce the level of an inflammatory response, for example, by lowering the subject's production of cytokines and/or chemokines. Example methods for co-administering prednisolone or prednisone with a gene therapy are described, for example, in International Patent Application Publication No. WO 2008/069942, the content of which is incorporated herein by reference, in its entirety, for all purposes.

In some embodiments, prednisolone or prednisone is administered to the human patient prior to administering the adeno-associated virus (AAV) particles, with the polynucleotide encoding the Factor VIII polypeptide, having high sequence identity to SEQ ID NO: 1 (CS04-FL-NA). For example, in some embodiments, prednisolone or prednisone is administered about a week, or about one or two days, before the AAV particles are administered to the patient. In some embodiments, a course of prednisolone or prednisone is administered starting about a week, or about one or two days, before the AAV particles are administered, and is continued after administration of the AAV particles.

In some embodiments, prednisolone or prednisone is co-administered to the human subject when administering the adeno-associated virus (AAV) particles with the polynucleotide encoding the Factor VIII polypeptide, having high sequence identity to SEQ ID NO: 1 (CS04-FL-NA). For example, in some embodiments, prednisolone or prednisone is administered on the same day, e.g., directly before or after administration of the AAV particles. In some embodiments, a course of prednisolone or prednisone is administered on the same day as the AAV particles are administered, and is continued after administration of the AAV particles.

In some embodiments, prednisolone or prednisone is administered to the patient after administering the adeno-associated virus (AAV) particles with the polynucleotide encoding the Factor VIII polypeptide, having high sequence identity to SEQ ID NO: 1 (CS04-FL-NA). For example, in some embodiments, prednisolone or prednisone is first administered about one or two days after AAV particles are administered to the patien.

It should be noted that prednisolone or prednisone is a small molecule drug that is administered orally (although it can also be administered intravenously), and thus “co-administration” in this context does not require that a single solution contains both drugs.

In some embodiments, the course of prednisolone or prednisone is administered to the patient over a period of at least two weeks, e.g., daily or every two days. In some embodiments, the course of prednisolone or prednisone is administered over a period of at least three weeks. In some embodiments, the dose of prednisolone or prednisone decreases during the course. For example, in one embodiment, the course begins with administration of about 60 mg of prednisolone or prednisone per day, and is reduced as the course progresses.

In one embodiment, the course includes administration of about 60 mg of prednisolone or prednisone per day to the human patient, during the first week of the course, administration of about 40 mg of prednisolone or prednisone per day to the patient, during the second week of the course, and administration of about 30 mg of prednisolone or prednisone per day to the patient, during the third week immediately following infusion of the AAV particles.

In some embodiments, the course includes further tapering administration of prednisolone or prednisone after the third week, e.g., administration of a tapering dose of prednisolone or prednisone. In one embodiment, the tapering dose of prednisolone or prednisone includes successively administering doses (e.g., one or more doses at each concentration) of about 20 mg prednisolone or prednisone per day, about 15 mg prednisolone or prednisone per day, about 10 mg prednisolone or prednisone per day, and about 5 mg prednisolone or prednisone per day.

In one embodiment, the tapering dose of prednisolone or prednisone includes administration of about 20 mg of prednisolone or prednisone per day to the patient, for 5 consecutive days (e.g., immediately) following completion of the initial course of prednisolone or prednisone, administration of about 15 mg of prednisolone or prednisone per day to the patient, for 3 consecutive days (e.g., immediately) following the 5 days on which the patient was administered 20 mg of prednisolone or prednisone, administration of about 10 mg of prednisolone or prednisone per day to the patient, for 3 consecutive days (e.g., immediately) following the 3 days on which the patientwas administered 15 mg of prednisolone or prednisone, and administration of about 5 mg of prednisolone or prednisone per day to the patient, for 3 consecutive days (e.g., immediately) following the 3 days on which the patientwas administered 10 mg of prednisolone or prednisone.

In one embodiment, the tapering dose of prednisolone or prednisone includes administration of about 30 mg of prednisolone or prednisone per day to the patient, for 7 consecutive days immediately following completion of the initial course of prednisolone or prednisone, administration of about 20 mg of prednisolone or prednisone per day to the patient, for 7 consecutive days immediately following the 7 days on which the patientwas administered 30 mg of prednisolone or prednisone, administration of about 15 mg of prednisolone or prednisone per day to the patient, for 5 consecutive days immediately following the 7 days on which the human subject was administered 20 mg of prednisolone or prednisone, administration of about 10 mg of prednisolone or prednisone per day to the patient, for 5 consecutive days immediately following the 5 days on which the patientwas administered 15 mg of prednisolone or prednisone, and administration of about 5 mg of prednisolone or prednisone per day to the patient, for 5 consecutive days immediately following the 5 days on which the patientwas administered 10 mg of prednisolone or prednisone.

In some embodiments, the length of a tapering dose of prednisolone or prednisone administered to the patient is determined based on whether the patient is still exhibiting signs of liver inflammation at the end of the initial course of prednisolone or prednisone (e.g., as indicated by a reduction in Factor VIII levels, e.g., Factor VIII titer or Factor VIII activity, or increase in liver enzymes).

For example, in one embodiment, a first level of Factor VIII (e.g., titer or activity) in the blood stream of the patient (e.g., in a blood sample collected from a patient) is determined following administration of adeno-associated virus (AAV) particles including a polynucleotide encoding a Factor VIII protein to the patient, and while the patient is receiving an initial course of glucocorticoid steroid treatment. A second level of Factor VIII (e.g., titer or activity) in the blood stream of the patient is determined after completion of the initial course of glucocorticoid steroid treatment. The second level of Factor VIII is then compared to the first level of Factor VIII. The patient is administered a first tapering dose of the glucocorticoid steroid over a time period of no more than three weeks when the second level of Factor VIII is not decreasing (e.g., when the second level of Factor VIII is not less than the first level of Factor VIII, or not less than a threshold amount below the first level of Factor VIII). The patient is administered a second tapering dose of the glucocorticoid steroid over a time period exceeding three weeks when the second level of Factor VIII is decreasing (e.g., when the second level of Factor VIII is less than the first level of Factor VIII, or less than a threshold amount below the first level of Factor VIII).

Similarly, in some embodiments, a first level of liver enzymes (e.g., a liver enzyme titer or activity) in the blood stream of the patient is determined prior to (e.g., or shortly after) administration of adeno-associated virus (AAV) particles including a polynucleotide encoding a Factor VIII protein to the patient. A second level of level of liver enzymes (e.g., a liver enzyme titer or activity) in the blood stream of the patient is determined after completion of the initial course of glucocorticoid steroid treatment. The second level of liver enzymes is then compared to the first level of liver enzymes. The patient is administered a first tapering dose of the glucocorticoid steroid over a time period of no more than three weeks when the second level of liver enzymes is not increasing (e.g., when the second level of liver enzymes is not greater than the first level of liver enzymes, or not more than a threshold amount above the first level of liver enzymes). The patient is administered a second tapering dose of the glucocorticoid steroid over a time period exceeding three weeks when the second level of liver enzymes is increasing (e.g., when the second level of liver enzymes is greater than the first level of liver enzymes, or more than a threshold amount above the first level of liver enzymes).

In some embodiments, the first tapering dose of prednisolone or prednisone includes administration of about 20 mg of prednisolone or prednisone per day to the patient, for 5 consecutive days (e.g., immediately) following completion of the initial course of prednisolone or prednisone, administration of about 15 mg of prednisolone or prednisone per day to the patient, for 3 consecutive days (e.g., immediately) following the 5 days on which the patientwas administered 20 mg of prednisolone or prednisone, administration of about 10 mg of prednisolone or prednisone per day to the patient, for 3 consecutive days (e.g., immediately) following the 3 days on which the human subject was administered 15 mg of prednisolone or prednisone, and administration of about 5 mg of prednisolone or prednisone per day to the patient, for 3 consecutive days (e.g., immediately) following the 3 days on which the patientwas administered 10 mg of prednisolone or prednisone.

In some embodiments, the second tapering dose of prednisolone or prednisone includes administration of about 30 mg of prednisolone or prednisone per day to the patient, for 7 consecutive days immediately following completion of the initial course of prednisolone or prednisone, administration of about 20 mg of prednisolone or prednisone per day to the patient, for 7 consecutive days immediately following the 7 days on which the patientwas administered 30 mg of prednisolone or prednisone, administration of about 15 mg of prednisolone or prednisone per day to the patient, for 5 consecutive days immediately following the 7 days on which the patientwas administered 20 mg of prednisolone or prednisone, administration of about 10 mg of prednisolone or prednisone per day to the patient, for 5 consecutive days immediately following the 5 days on which the patientwas administered 15 mg of prednisolone or prednisone, and administration of about 5 mg of prednisolone or prednisone per day to the patient, for 5 consecutive days immediately following the 5 days on which the patientwas administered 10 mg of prednisolone or prednisone.

In some embodiments, the course of prednisolone or prednisone is administered after detecting an indication of an immune reaction in the patient, following administration of the AAV particles. In some embodiments, the course of prednisolone or prednisone is administered after detecting an indication of liver inflammation in the patient. For example, in some embodiments, the patient is monitored for liver inflammation following administration of the AAV particles, and the patient is administered a course of prednisolone or prednisone upon detecting liver inflammation.

In some embodiments, a rapid or large decrease in Factor VIII expression or Factor VIII activity in the blood stream of the patient indicates liver inflammation in the subject. In some embodiments, it is possible that an early peak of Factor VIII activity may be observed followed by a small and/or gradual decrease, after which the Factor VIII protein may be made at a somewhat lower level, which does not require administration of a course of prednisolone or prednisone. For example, in some embodiments, the amount of Factor VIII (e.g., a Factor VIII titer or Factor VIII activity level) in the patient blood stream is monitored following administration of the AAV particles, and the subject is administered a course of prednisolone or prednisone if a rapid or large decrease in the amount of Factor VIII (e.g., more than a threshold decrease in the Factor VIII titer or Factor VIII activity level, as compared to a level in the patient blood stream following administration of the AAV particles) is detected.

In some embodiments, an increase in the level of liver enzymes in the patient indicates liver inflammation in the subject. For example, in some embodiments, the level of liver enzymes in the patient is monitored following administration of the AAV particles, and the subject patient is administered a course of prednisolone or prednisone if an increase in the level of liver enzymes (e.g., more than a threshold increase in the amount of liver enzymes, e.g., as compared to a baseline level of liver enzymes in the patient before administration of the AAV particles or shortly after administration of the AAV particles) is detected.

### Post-Administration Monitoring

In some embodiments, methods are provided for monitoring a patient for adverse reactions and/or treatment efficacy, following administration of adeno-associated virus (AAV) particles with a polynucleotide encoding a Factor VIII polypeptide, e.g., polynucleotides having high sequence identity to SEQ ID NO: 1 (CS04-FL-NA). In some embodiments, the patient is monitored for one or more of (a) an indication of liver inflammation (e.g., via rapid or large decreases in Factor VIII levels (e.g., titer or activity) and/or increases in liver enzymes (e.g., titer or activity)), (b) an increase in Factor VIII inhibitor antibodies in the patient's blood stream, (c) an increase in capsid proteins in the patient's blood stream, (d) an increase in anti-capsid protein antibodies in the patient's blood stream, and (e) an increase in polynucleotides, or fragments thereof, encoding the Factor VIII polypeptide in the patient's blood stream. In some embodiments, the subject is further treated upon detection of one or more adverse reaction and/or inefficacy of the treatment.

For example, in one embodiment, a method is provided for monitoring the efficacy of Factor VIII gene therapy of hemophilia A using adeno-associated virus (AAV) particles that include a polynucleotide encoding a Factor VIII polypeptide. The method includes determining whether Factor VIII inhibitor antibodies are present in the blood stream of the patient (e.g., in a blood sample collected from the patient) after administration of the AAV particles to the patient. In some embodiments, when Factor VIII inhibitor antibodies are detected in the blood stream of the patient (e.g., when an increase in the level of Factor VIII inhibitor antibodies is detected, as compared to a level in the patient prior to administration of the AAV particles), the method includes administering an alternative agent for treatment of hemophilia A to the patient.

In some embodiments, the alternative agent for treatment of hemophilia A is an alternative form of Factor VIII (e.g., one that does not include, or masks, one of more epitopes targeted by the detected Factor VIII inhibitor antibodies). In some embodiments, the alternative form of Factor VIII is a chemically-modified Factor VIII protein (e.g., a chemically-modified human or porcine Factor VIII protein). In some embodiments, the alternative form of Factor VIII is a Factor VIII protein derived from a non-human Factor VIII protein, e.g., a porcine Factor VIII protein. In some embodiments, the alternative agent for treatment of hemophilia A is a Factor VIII bypass therapy, e.g., a therapeutic agent that includes Factor II, Factor IX, and Factor X. For example, in some embodiments, the Factor VIII bypass therapy is a Factor VIII Inhibitor Bypass Activity (FEIBA) complex, recombinant activated factor VII (FVIIa), a prothrombin complex concentrate, or an activated prothrombin complex concentrate.

In one embodiment, a method is provided for monitoring the level of polynucleotide encoding a Factor VIII polypeptide, or a fragment thereof, in the blood stream of the patient following administration of the AAV particles. In one embodiment, the method includes administering to a hemophilia A patient a dose of adeno-associated virus (AAV) particles per kilogram body weight of the patient, where the AAV particles include a polynucleotide encoding a Factor VIII protein at a first time point. The method also includes measuring the level polynucleotide encoding the Factor VIII protein, or fragments thereof, in the patient's blood stream at a later time point, where the later time point is 7 days or longer. In one embodiment, the method includes administering to a hemophilia A patient a dose of 2×10¹² adeno-associated virus (AAV) particles per kilogram body weight of the patient, where the AAV particles include a polynucleotide having a nucleic acid sequence of SEQ ID NO: 1 (CS04-FL-NA) at a first time point. The method also includes measuring the level nucleic acids of SEQ ID NO: 1, or fragments thereof, in the patient's blood stream at a later time point, where the later time point is 7 days or longer. In one embodiment, the method includes administering to a hemophilia A patient a dose of 6×10¹² adeno-associated virus (AAV) particles per kilogram body weight of the patient, where the AAV particles include a polynucleotide having a nucleic acid sequence of SEQ ID NO: 1 (CS04-FL-NA) at a first time point. The method also includes measuring the level nucleic acids of SEQ ID NO: 1, or fragments thereof, in the patient's blood stream at a later time point, where the later time point is 7 days or longer. In some embodiments of the method, the later time point is at least 14 days later or at least 21 days later. In some embodiments, the later time point is at 7 days, 14 days, or 21 days after administration of the AAV particles.

In one embodiment, a method is provided for monitoring the level of capsid protein in the blood stream of the patient following administration of the AAV particles. In one embodiment, the method includes administering to a hemophilia A patient a dose of 2×10¹² adeno-associated virus (AAV) particles per kilogram body weight of said patient, where the AAV particles include a capsid protein and a polynucleotide that includes a nucleic acid sequence of SEQ ID NO: 1 (CS04-FL-NA) at a first time point. The method also includes measuring the level of the capsid protein in said patient's blood stream at a later time point, where the later time point is 7 days or longer. In one embodiment, the method includes administering to a hemophilia A patient a dose of 6×10¹² adeno-associated virus (AAV) particles per kilogram body weight of the patient, where the AAV particles include a capsid protein and a polynucleotide that includes a nucleic acid sequence of SEQ ID NO: 1 (CS04-FL-NA) at a first time point. The method also includes measuring the level of the capsid protein in said patient's blood stream at a later time point, where the later time point is 7 days or longer. In one embodiment, the method includes administering to a hemophilia A patient a dose of adeno-associated virus (AAV) particles per kilogram body weight of said patient, where the AAV particles include a capsid protein and a polynucleotide that encodes a Factor VIII protein at a first time point. The method also includes measuring the level of the capsid protein in said patient's blood stream at a later time point, where the later time point is 7 days or longer. In some embodiments of the method, the later time point is at least 14 days later or at least 21 days later. In some embodiments, the later time point is at 7 days, 14 days, or 21 days after administration of the AAV particles.

In one embodiment, a method is provided for monitoring the level of Factor VIII inhibitor antibodies in the blood stream of the patient following administration of the AAV particles. In one embodiment, the method includes administering to a hemophilia A patient a dose of 2×10¹² adeno-associated virus (AAV) particles per kilogram body weight of the patient, where the AAV particles include a polynucleotide that includes a nucleic acid sequence of SEQ ID NO: 1 (CS04-FL-NA) at a first time point. The method also includes measuring the level of anti-Factor VIII antibodies in the patient's blood stream at a later time point, wherein the later time point is 7 days or longer. In one embodiment, the method includes administering to a hemophilia A patient a dose of 6×10¹² adeno-associated virus (AAV) particles per kilogram body weight of the patient, where the AAV particles include a polynucleotide that includes a nucleic acid sequence of SEQ ID NO: 1 (CS04-FL-NA) at a first time point. The method also includes measuring the level of anti-Factor VIII antibodies in the patient's blood stream at a later time point, wherein said later time point is 7 days or longer. In one embodiment, the method includes administering to a hemophilia A patient a dose of adeno-associated virus (AAV) particles, where the AAV particles include a polynucleotide that encodes a Factor VIII protein at a first time point. The method also includes measuring the level of anti-Factor VIII antibodies in the patient's blood stream at a later time point, wherein said later time point is 7 days or longer. In some embodiments of the method, the later time point is at least 14 days later or at least 21 days later. In some embodiments, the later time point is at 7 days, 14 days, or 21 days after administration of the AAV particles.

In one embodiment, a method is provided for monitoring the level of anti-capsid protein antibodies in the blood stream of the subject following administration of the AAV particles. In one embodiment, the method includes administering to a hemophilia A patient a dose of 2×10¹² adeno-associated virus (AAV) particles per kilogram body weight of said patient, where the AAV particles include a capsid protein and a polynucleotide that includes a nucleic acid sequence of SEQ ID NO: 1 (CS04-FL-NA) at a first time point. The method also includes measuring the level of anti-capsid protein antibodies in the patient's blood stream at a later time point, where the later time point is 7 days or longer. In one embodiment, the method includes administering to a hemophilia A patient a dose of 6×10¹² adeno-associated virus (AAV) particles per kilogram body weight of the patient, where the AAV particles include a capsid protein and a polynucleotide that includes a nucleic acid sequence of SEQ ID NO: 1 (CS04-FL-NA) at a first time point. The method also includes measuring the level of anti-capsid protein antibodies in said patient's blood stream at a later time point, where the later time point is 7 days or longer. In one embodiment, the method includes administering to a hemophilia A patient a dose of adeno-associated virus (AAV) particles per kilogram body weight of the patient, where the AAV particles include a capsid protein and a polynucleotide that encodes a Factor VIII protein at a first time point. The method also includes measuring the level of anti-capsid protein antibodies in said patient's blood stream at a later time point, where the later time point is 7 days or longer. In some embodiments of the method, the later time point is at least 14 days later or at least 21 days later. In some embodiments, the later time point is at 7 days, 14 days, or 21 days after administration of the AAV particles.

### IV. EXAMPLES

Example 1 Construction of a Codon-Altered Factor VIII Variant Expression Sequence

Two hurdles had to be overcome in order to create a Factor VIII coding sequence that is effective for gene therapy of hemophilia A. First, because of the genomic size limitations of conventional gene therapy delivery vectors (e.g., AAV virions), the encoded Factor VIII polypeptide had to be shortened considerably. Second, the coding sequence had to be altered to: (i) stabilize packaging interactions within the delivery vector, (ii) stabilize the mRNA intermediary, and (iii) improve the robustness of transcription/translation of the mRNA.

To achieve the first objective, Applicants started with a B-domain deleted Factor VIII variant construct, referred to herein as “FVIII-BDD-SQ.” In this construct, the B-domain is replaced with a fourteen amino acid sequence referred to as the “SQ” sequence. Recombinant FVIII-BDD-SQ is sold under the trade name REFACTOR, and has been shown to be effective for the management of hemophilia A. However, the native coding sequence for FVIII-BDD-SQ, which includes human wild-type nucleic acid sequences for the Factor VIII heavy and light chains, is ineffectively expressed in gene therapy vectors.

To address the poor expression of the native FVIII-BDD-SQ, the codon optimization algorithm described in Fath et al. (PLOS ONE, 6:e17596 (2011)), modified as described in Ward et al. (Blood, 117:798 (2011)) and in McIntosh et al. (Blood, 121, 3335-3344 (2013)) was applied to the FVIII-BDD-SQ sequence to create first intermediate coding sequence CS04a. However, Applicants recognized that the CS04a sequence created using the modified algorithm could be improved by further modifying the sequence. Accordingly, Applicants re-introduced CpG dinucleotides, re-introduced the CGC codon for arginine, changed the leucine and serine codon distributions, re-introduced highly conserved codon pairs, and removed cryptic TATA box, CCAAT box, and splice site elements, while avoiding CpG islands and local overrepresentation of AT-rich and GC-rich stretches.

First, the modified algorithm systematically replaces codons containing CpG-dinucleotides (e.g., arginine codons) with non-CpG-dinucleotide codons, and eliminates/avoids CpG-dinucleotides created by neighboring codons. This strict avoidance of CpG dinucleotides is usually done to prevent TLR-induced immunity after intramuscular injection of DNA vaccines. However, doing so limits the codon optimization possibilities. For example, the modified algorithm excludes use of the complete set of CGX arginine codons. This is particularly disruptive in the coding of genes for expression in human cells, because CGC is the most frequently used arginine codon in highly expressed human genes. Additionally, avoiding the creation of CpGs by neighboring codons further limits the optimization possibilities (e.g., limits the number of codon pairs that may be used together).

Because TLR-induced immunity is not expected to be a problem associated with liver-directed, AAV-based gene therapy, codons including CpGs, and neighboring codons creating CpGs, were re-introduced into intermediate coding sequence CS04a, preferentially in the sequence coding for the Factor VIII light chain (e.g., at the 3′ end of the FVIII-BDD-SQ coding sequence). This allowed for more frequent use of preferred human codons, particularly those for arginine. Care was taken, however, to avoid creation of CpG islands, which are regions of coding sequence having a high frequency of CpG sites. This is contrary to the teachings of Krinner et al. (Nucleic Acids Res., 42(6):3551-64 (2014)), which suggests that CpG domains downstream of transcriptional start sites promote high levels of gene expression.

Second, the modified algorithm applies certain codons exclusively, such as CTG for leucine, GTG for valine, and CAG for glutamine. However, this offends the principles of balanced codon use, for example, as proposed in Haas et al. (Current Biology, 6(3):315-24 (1996)). To account for the overuse of preferred codons by the modified algorithm, alternate leucine codons were re-introduced where allowed by the other rules applied to the codon alteration (e.g., CpG frequency and GC content).

Third, the modified algorithm replaces codon pairs without regard to how conserved they are in nature, when certain criteria (e.g., the presence of CG-dinucleotides) are met. To account for beneficial properties which may have been conserved by evolution, the most conserved codon pairs that were replaced by the algorithm and the most conserved preferred codon pairs, e.g., as described in Tats et al. (BMC Genomics 9:463 (2008)), were analyzed and adjusted where allowed by the other rules applied to the codon alteration (e.g., CpG frequency and GC content).

Fourth, serine codons used in the intermediate coding sequence were also re-engineered. Specifically, AGC, TCC, and TCT serine codons were introduced into the modified coding sequence with higher frequency, to better match overall for human codon usage (Haas et al., supra).

Fifth, TATA box, CCAAT box elements, and intron/exon splice sites were screened and removed from the modified coding sequence. When modifying the coding sequence, care was taken to avoid local overrepresentation of AT-rich or GC rich stretches.

Finally, in addition to optimizing the codon usage within the coding sequence, the structural requirements of the underlying AAV virion were considered when further refining the intermediate coding sequence CS04a. AAV vectors (e.g., the nucleic acid portion of an AAV virion) are packaged as single stranded DNA molecules into their capsids (for review, see, Daya and Berns, Clin. Microbiol Rev., 21(4):583-93 (2008)). The GC content of the vector is therefore likely to influence packaging of the genome and, thus, vector yields during production. Like many algorithms, the modified algorithm used here creates an optimized gene sequence with a GC content of at least 60% (see, Fath et al., PLOS One, 6(3):e17596 (2011) (erratum in: PLOS One, (6)3 (2011)). However, the AAV8 capsid protein is encoded by a nucleotide sequence having a lower GC content of about 56%. Thus, to better mimic the native AAV8 capsid protein coding sequence, the GC content of the intermediate coding sequence CS04a was reduced to 56%.

The resulting CS04 coding sequence, shown in FIG. 2, has an overall GC content of 56%. The CpG-dinucleotide content of the sequence is moderate. However, CpG dinucleotides are predominantly in the downstream portion of the coding sequence, e.g., the portion coding for the Factor VIII light chain. The CS04 sequence has 79.77% nucleotide sequence identity to the corresponding coding sequences in wild-type Factor VIII (Genbank accession M14113).

For comparison purposes, several other codon-optimized, ReFacto constructs were prepared. The CS08 ReFacto construct was codon-optimized as described in Radcliff P.M. et al., Gene Therapy, 15:289-97 (2008), the content of which is hereby expressly incorporated by reference herein, in its entirety, for all purposes. The CS10 codon-optimized ReFacto construct was obtained from Eurofins Genomics (Ebersberg, Germany). The CS11 codon-optimized ReFacto construct was obtained from Integrated DNA Technologies, Inc. (Coralville, USA). The CH25 codon-optimized ReFacto construct was obtained from ThermoFischer Scientific's GeneArt services (Regensburg, Germany). The CS40 ReFacto construct consists of the wild type Factor VIII coding sequence. The sequence identities shared between each of the ReFacto coding sequences is shown in Table 2, below.

TABLE 2
Percent identity matrix for codon-altered Factor VIII constructs.

| | CS04 | CS08 | CS10 | CS11 | CS40 | CH25 |
|---|---|---|---|---|---|---|
| CS04 | 100% | | | | | |
| CS08 | 82.2% | 100% | | | | |
| CS10 | 79.4% | 78.4% | 100% | | | |
| CS11 | 78.3% | 78.1% | 77.5% | 100% | | |
| CS40 | 79.8% | 76.7% | 77.6% | 75.4% | 100% | |
| CH25 | 85.1% | 85.0% | 79.9% | 79.4% | 75.8% | 100% |

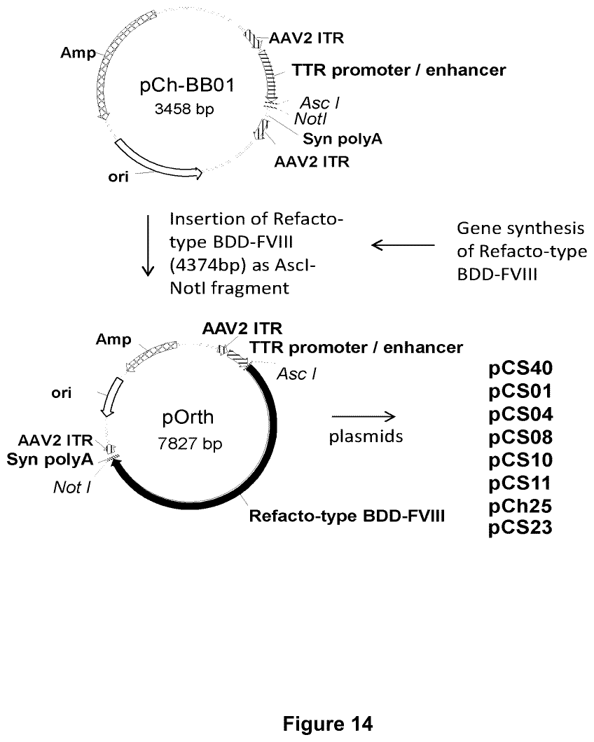

Plasmids of each construct were constructed by cloning different synthetic DNA fragments into the same vector backbone plasmid (pCh-BB01). DNA synthesis of the Refacto-type BDD-FVIII fragments with flanking AscI and NotI enzyme restriction sites were done by ThermoFischer Scientific (Regensburg, Germany). The vector backbone contains two flanking AAV2-derived inverted terminal repeats (ITRs) that encompass a promoter/enhancer sequence derived from the liver-specific murine transthyretin gene, AscI and NotI enzyme restriction sites for insertion of the respective Refacto-type BDD-FVIII and a synthetic poly A site. After ligation of the prepared vector backbone and inserts via the AscI and NotI sites, the resulting plasmids were amplified in milligram scale. The Refacto-type BDD-FVIII sequences of the constructs were verified by direct sequencing (Microsynth, Balgach, Switzerland). The cloning resulted in seven different plasmid constructs named pCS40, pCS04, pCS08, pCS10, pCS11, and pCh25 (FIG. 14). The constructs have the same vector backbone and encode the same B-domain deleted FVIII protein (Refacto-type BDD-FVIII), but differ in their FVIII coding sequence.

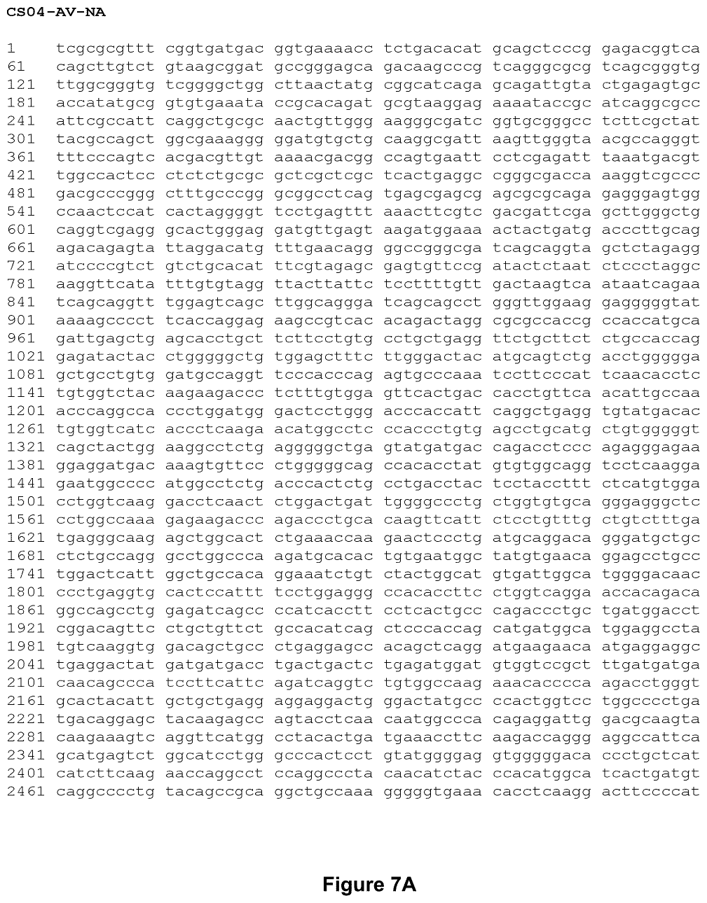

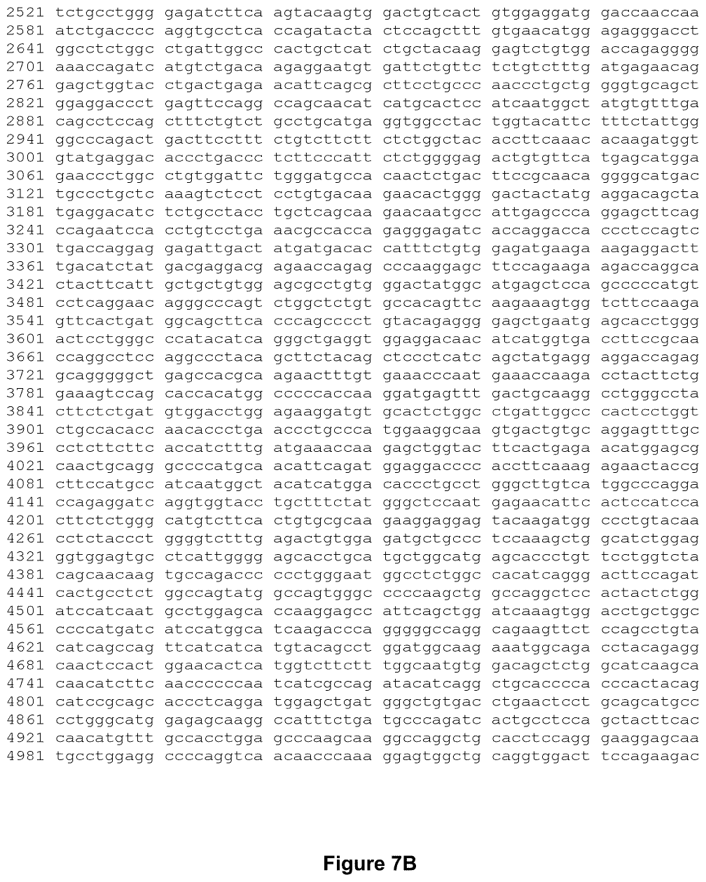

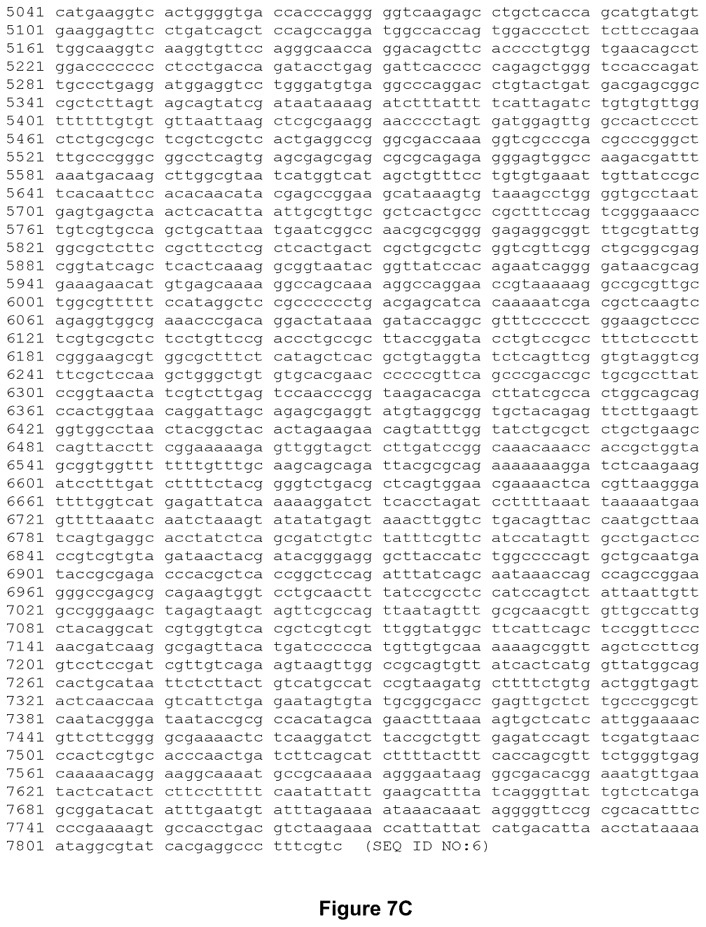

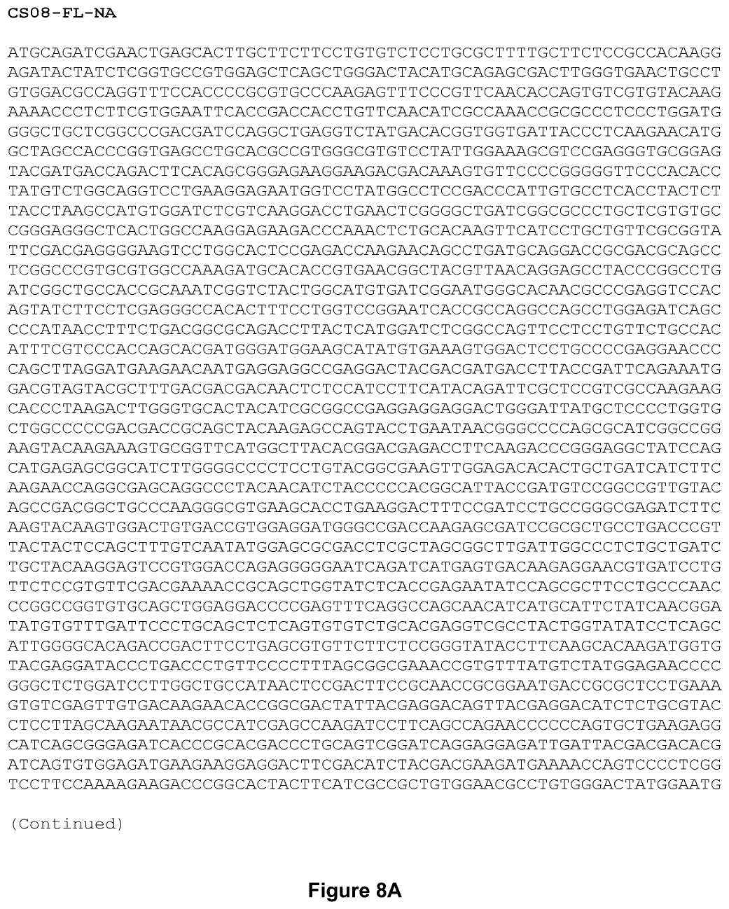

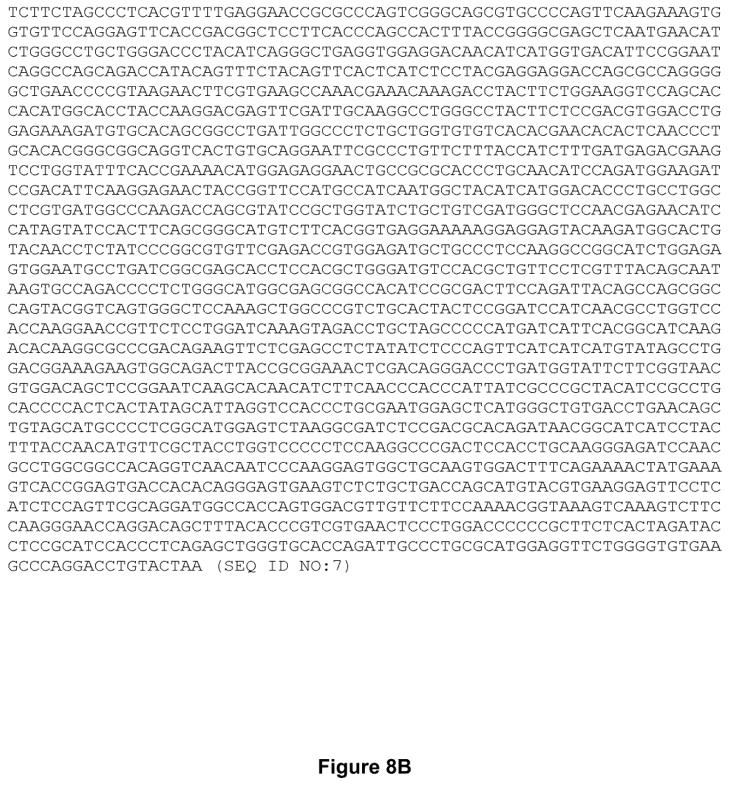

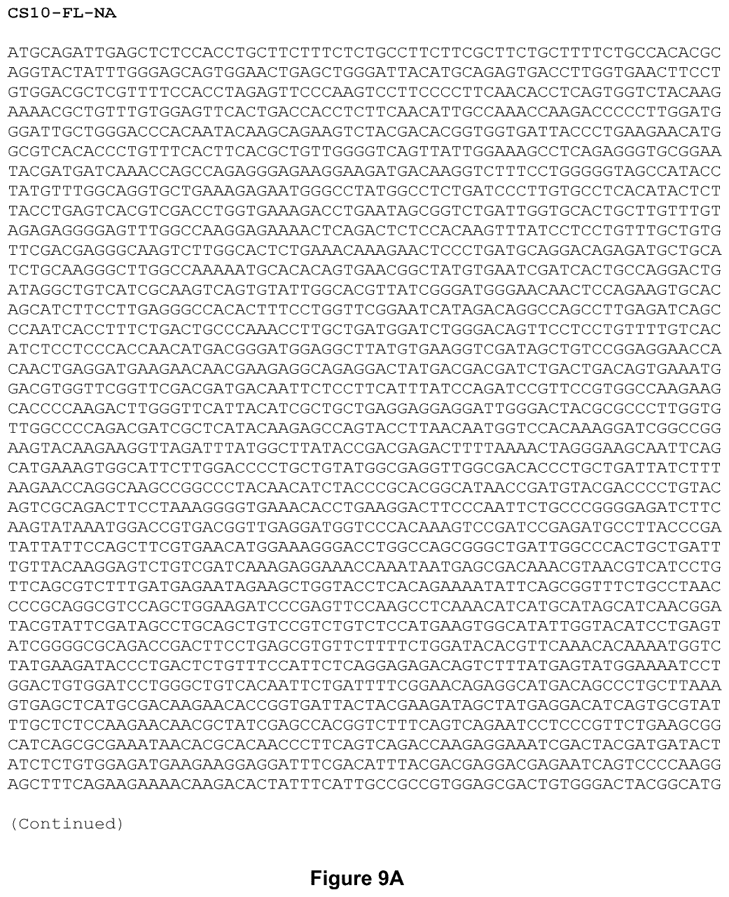

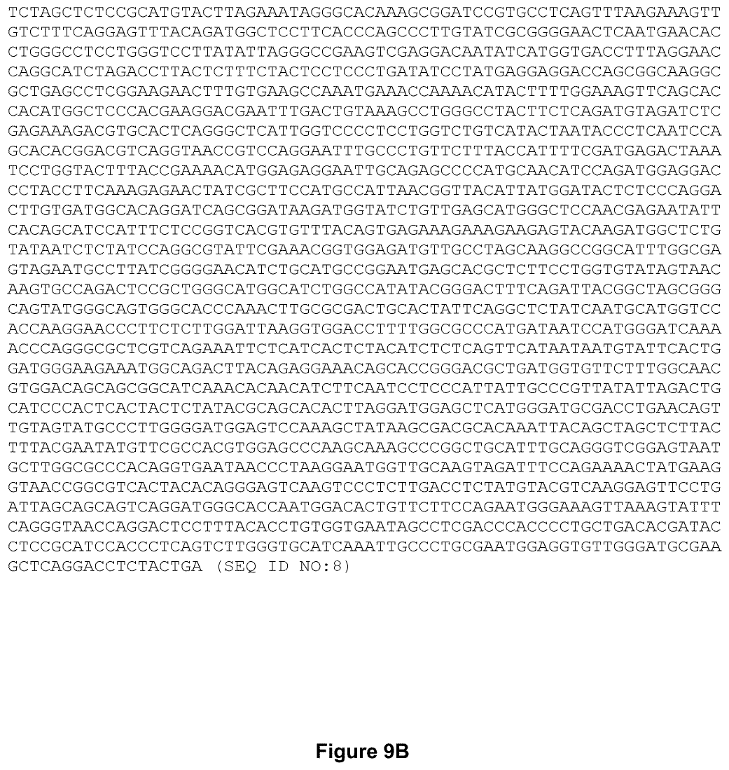

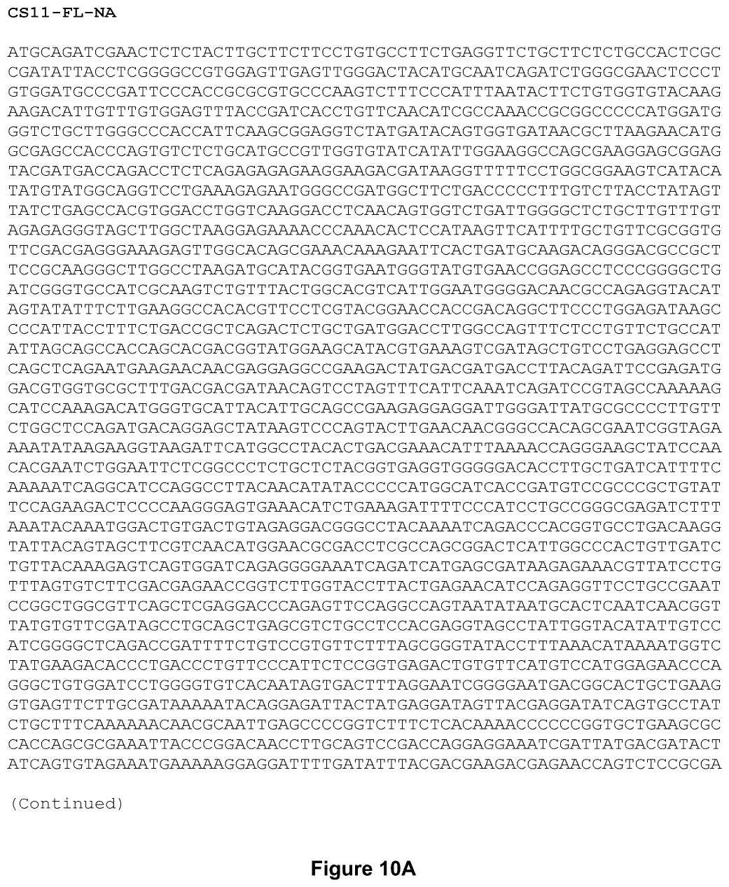

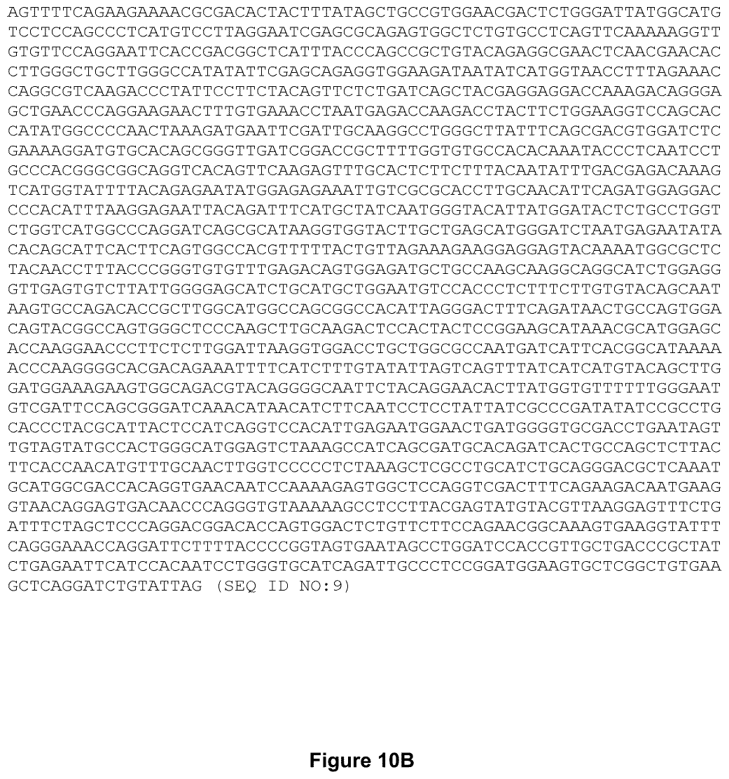

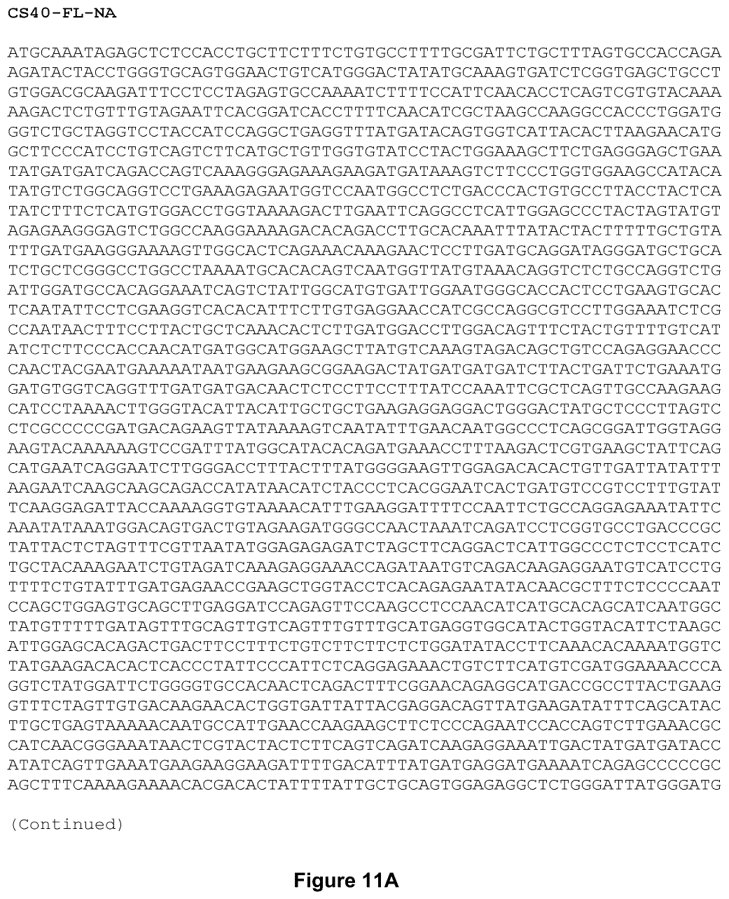

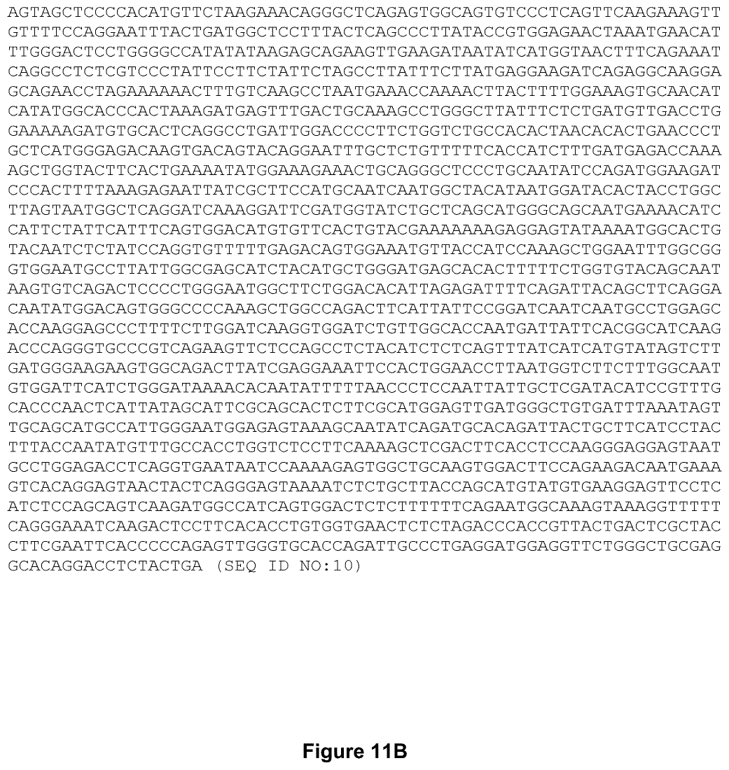

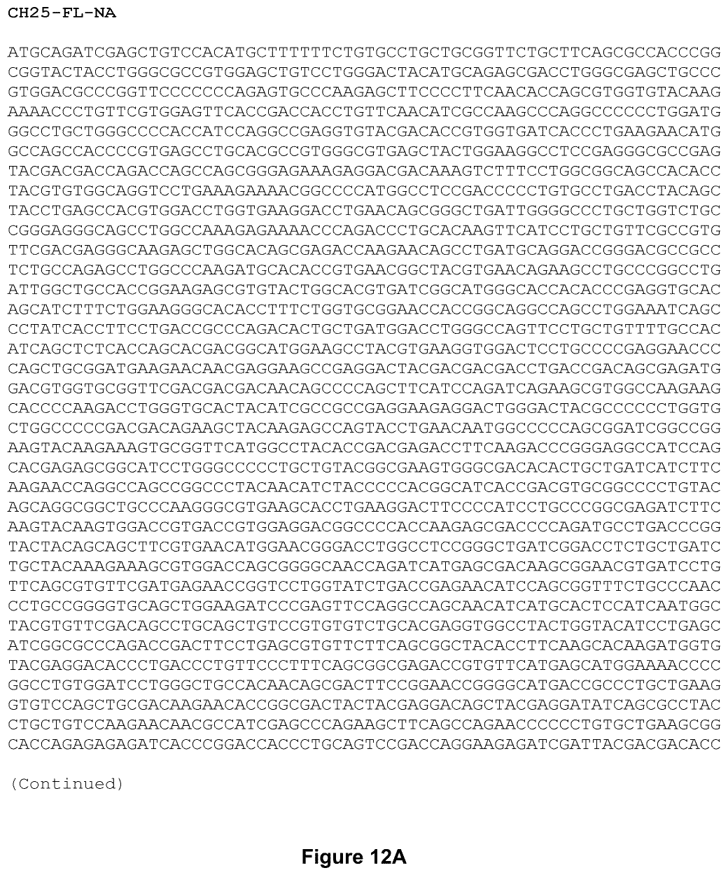

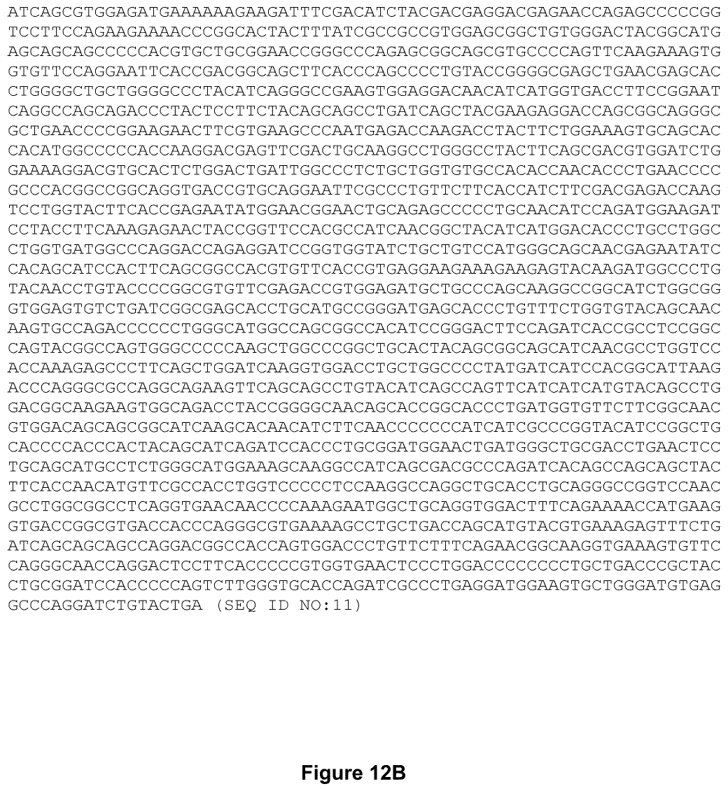

AAV8-based vectors were prepared by the three plasmid transfection method, as described in Grieger JC, et al. (Virus Vectors Using Suspension HEK293 Cells and Continuous Harvest of Vector From the Culture Media for GMP FIX and FLTI Clinical Vector, Mol Ther., October 6. (2015) doi: 10.1038/mt.2015.187. [Epub ahead of print]), the content of which is hereby expressly incorporated by reference herein, in its entirety, for all purposes. HEK293 suspensions cells were used for plasmid transfections using the corresponding FVIII vector plasmid, the helper plasmid pXX6-80 (carrying adenoviral helper genes), and the packaging plasmid pGSK2/8 (contributing the rep2 and cap8 genes). To isolate the AAV8 constructs, the cell pellets of one liter cultures were processed using iodixanol gradients, as described in Grieger et al. (2015, Supra). The procedure resulted in vector preparations called vCS04, vCS08, vCS10, vCS11, and vCH25. Vectors were quantified by qPCR using the universal qPCR procedure targeting the AAV2 inverted terminal repeats (Aurnhammer, Human Gene Therapy Methods: Part B 23:18-28 (2012)). A control vector plasmid carrying AAV2 inverted terminal repeats served for preparing the standard curve. The resulting vCS04 construct is presented as SEQ ID NO: 8 in FIGS. 7A-7C.

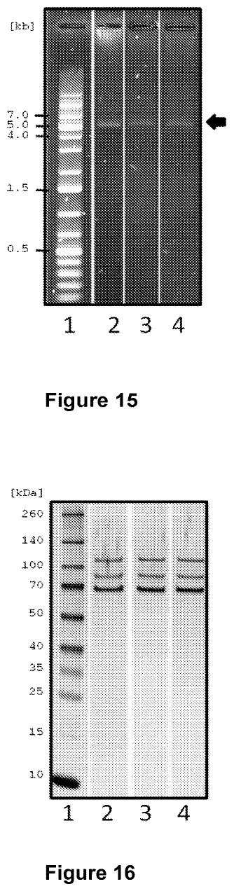

The integrity of the vector genomes was analyzed by AAV agarose gel electrophoresis. The electrophoresis was performed as described in Fagone et al., Human Gene Therapy Methods 23:1-7 (2012). Briefly, AAV vector preparations were incubated at 75° C. for 10 minutes in the presence of 0.5% SDS and then cooled down to room temperature. Approximately 1.5E10 vector genomes (vg) were loaded per lane on a 1% 1×TAE agarose gel and electrophoresed for 60 min at 7 V/cm of gel length. The gel was then stained in 2×GelRed (Biotium Cat #41003) solution and imaged by ChemiDocTMMP (Biorad). The results shown in FIG. 15 demonstrate that the vCS04 and vCS40 viral vectors have the same-sized genome, indicated by a distinct band in the 5 kb range (FIG. 15, lanes 2-4). Despite a vector size of approx. 5.2 kb, the genome is a homogenous band confirming correct packaging of the somewhat oversized genome (relative to an AAV wild-type genome of 4.7 kb). All other vCS vector preparations show the same genomic size (data not shown).

In order to confirm the expected pattern of capsid proteins, SDS PAGE followed by silver staining was performed with the vectors vCS04 and vCS40 (FIG. 16). As shown in the figure, the downstream purification procedure resulted in highly purified material displaying the expected protein pattern of VP1, VP2 and VP3 (FIG. 16, lanes 2-4). The same pattern was seen with all other viral preparations (not shown). The SDS-PAGE procedure of AAV preparations was done according to standard procedures. Each lane contained 1E10 vg of the respective viral construct, and were separated on a 4-12% Bis-Tris (NuPAGE® Novex, Life Technologies) gel as per manufacturer's instructions. Silver staining was performed with a SilverQuest™ kit (Novex, Life Technologies) according to the manufacturer's instructions.

Surprisingly, AAV vector vCS04 had higher virion packaging, measured by higher yields in AAV virus production, as compared to the vCS40 wild-type coding construct and the other codon-optimized constructs. As shown in Table 3, the vCS04 vector replicated substantially better than vCS40, providing a 5-7 fold yield increase in AAV titer.

TABLE 3
Yields per liter cell culture obtained with AAV vector
constructs vCS04 and vCD40, as purified from cell pellets.

| | Vector | | |
|---|---|---|---|
| | concentration | Yields | Fold |
| | [vg/ml] | [vg/liter] | increase |
| Construct | ×10E12 | ×10E12 | vs wt |
| vCS40 | 2.0 | 11.0 | — |
| vCS04-Sample 1 | 17.6 | 79.2 | 7.2 |
| vCS04-Sample 2 | 15.9 | 58.8 | 5.4 |

### Example 2—In Vivo Expression of Codon-Altered Factor VIII Variant Expression Sequences

To test the biological potency of the codon-altered Factor VIII variant sequences, the ReFacto-type FVIII constructs described in Example 1 were administered to mice lacking Factor VIII. Briefly, the assays were performed in C57B1/6 FVIII knock-out (ko) mice (with 6-8 animals per group) by tail vein injection of 4E12 vector genomes (vg) per kilogram body weight of mouse. Blood was drawn 14 days after injection by retroorbital puncture and plasma was prepared and frozen using standard procedures. Expression levels at day 14 were chosen because there is minimal influence of inhibitory antibodies at this time, which are seen in some animals of this mouse model at later times. FVIII activity in the mouse plasma was determined using the Technochrome FVIII assay performed, with only minor modifications, as suggested by the manufacture (Technoclone, Vienna, Austria). For the assay, the plasma samples were appropriately diluted and mixed with assay reagents, containing thrombin, activated factor IX (FIXa), phospholipids, factor X and calcium. Following FVIII activation by thrombin a complex with FIXa, phospholipids and calcium is formed. This complex activates FX to activated FX (FXa) which in turn cleaves para-nitroanilide (pNA) from the chromogenic substrate. The kinetics of pNA formation is measured at 405 nm. The rate is directly proportional to the FVIII concentration in the sample. FVIII concentrations are read from a reference curve and results are given in IU FVIII/milliliter.

The results, presented in Table 4 below, demonstrate that the codon-altered sequences designed using commercial algorithms (CS10, CS11, and CH25) provided only a modest increase in BDD-Factor VIII (3-4 fold) as compared to the wild-type BDD-Factor VIII construct (CS40). Similarly, the codon-altered BDD-Factor VIII construct prepared as described in Radcliffe et al. (CS08), only provided a 3-4 fold increase in BDD-FVIII expression. This result is consistent with the results reported in Radcliff et al. Surprisingly, the CS04 construct provided much higher BDD-FVIII expression in the in-vivo biopotency assays (e.g., a 74-fold increase).

TABLE 4
Expression of FVIII in the plasma of FVIII-knock-out mice induced
by the different AAV vector constructs.

| | | Average FVIII | | | |
|---|---|---|---|---|---|
| | | Expression | | Number | Fold |
| | Codon | at Day | Standard | of | increase |
| Construct | Algorithm | 14 [IU/ml] | deviation | mice | vs wt |
| vCS40 | Human | 0.03 | 0.03 | 12 | — |
| | wild-type | | | | |
| vCS04 | Applicants’ | 2.21 | 1.20 | 55 | 73.7 |
| vCS08 | Radcliffe | 0.11 | 0.01 | 6 | 3.6 |
| | et al. | | | | |
| vCS10 | Eurofins | 0.09 | 0.01 | 7 | 3.0 |
| vCS11 | IDT | 0.08 | 0.02 | 8 | 2.7 |
| vCH25 | GeneArt | 0.13 | 0.12 | 18 | 4.3 |

### Example 3—Non-Clinical Efficacy and Toxicology Evaluation of a Human FVIII Gene Therapy Vector in Mice

Hemophilia A is an inherited bleeding disorder caused by missing or defective factor VIII (FVIII) and treated with plasma-derived or recombinant factor concentrates. These concentrates need to be infused on a regular basis to maintain adequate FVIII levels to control and prevent bleeding events. Given the challenges of protein replacement therapy, gene therapy may offer an alternative therapeutic approach for patients with hemophilia A. By introducing a functional F8 gene copy into the target hepatic cells to induce endogenous FVIII expression, frequent infusions of clotting factor may no longer be necessary.

Adeno-associated virus (AAV)-based gene therapy has the potential to provide clinical benefit in patients with hemophilia A. A recombinant (r) AAV8-based gene therapy vector containing the CS04 Factor VIII codon optimized construct is designed to deliver a human codon-optimized B-domain-deleted FVIII (BDDFVIII) transgene under the control of a liver-specific transthyretin promoter. This construct was used to examine the dose-response relationship for FVIII activity in F8 knockout (ko) mice and to evaluate toxicity following a single intravenous administration.

Briefly, to test the efficacy of the treatment, 12 male FVIII knock-out mice per group were administered a single intravenous dose of 3.0×10¹¹, 1.2×10¹², or 3.0×10¹² of the vector capsid particles (cp)/kg or 10 mL/kg buffer. Retro-orbital blood samples were taken every other week over 8 weeks and analyzed for FVIII using a chromogenic assay. The plasma samples obtained from the final in-life blood sampling were also used for the analysis of FVIII binding and neutralizing antibodies. At the end of the observation period, hemostatic control was assessed using a tail-tip bleeding assay.

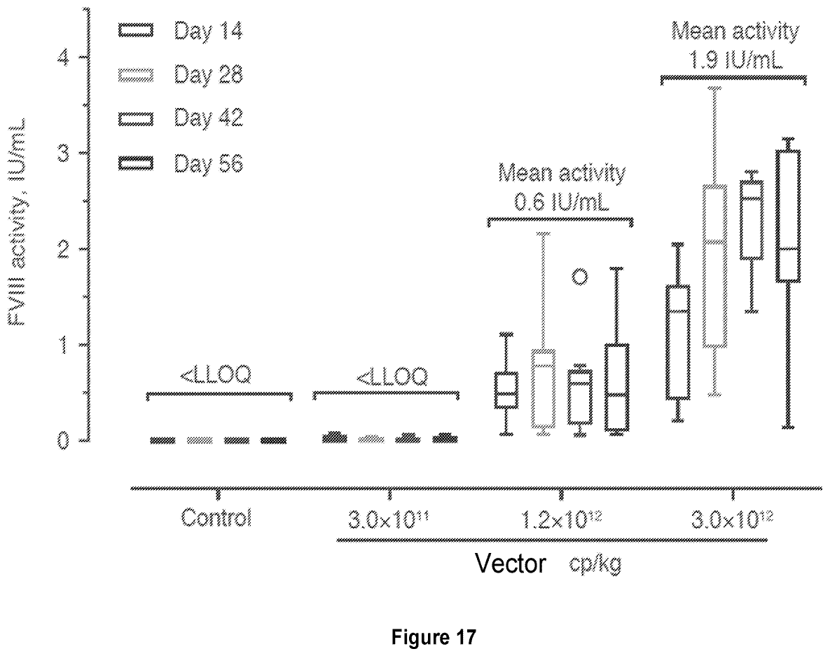

At study end, all samples were negative for anti-BDD-FVIII binding antibodies with the exception of 4 animals (treated with 3.0×10¹² cp/kg vector) that tested positive for binding and neutralizing antibodies. These animals were excluded from statistical analysis of FVIII activity levels and blood loss in the tail-tip bleeding assay. The administration of 1.2×10¹² or 3.0×10¹² cp/kg vector resulted in a dose-dependent increase in mean plasma FVIII activity to 0.6 and 1.9 IU/mL, respectively, calculated over the period of investigation, but FVIII activity was below the lower limit of quantification (LLOQ) in mice treated with buffer or 3.0×10¹¹ cp/kg vector (FIG. 17).

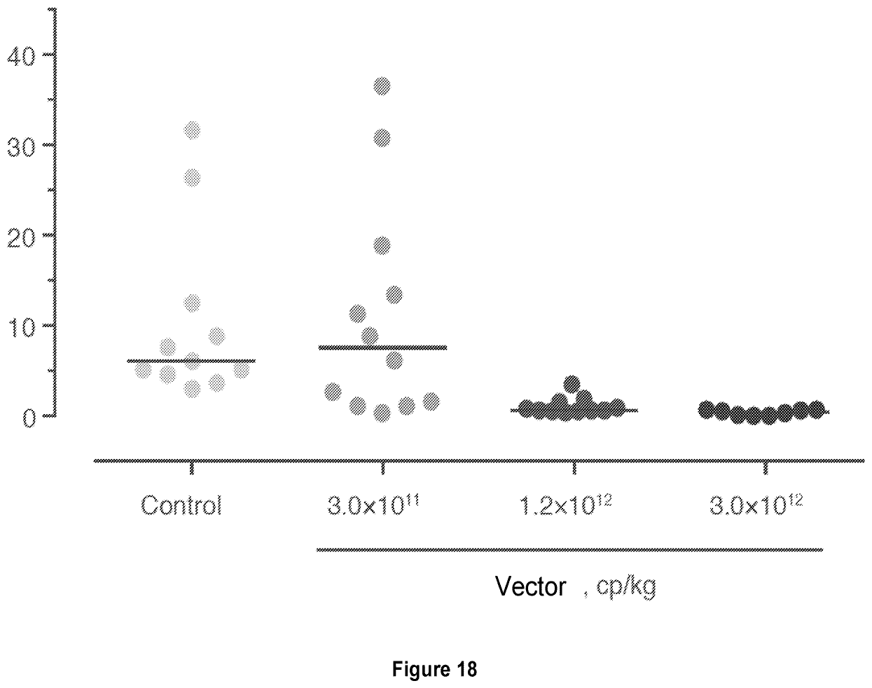

Efficacy was assessed in a tail-tip bleeding assay on Day 63. Blood loss over 60 minutes in mg/g body weight is presented in FIG. 18. Animals treated with buffer or 3.0×10¹¹ cp/kg of the gene therapy vector showed similar blood loss (6.1 mg/g and 7.5 mg/g, respectively), consistent with the absence of detectable FVIII activity. Higher doses of the gene therapy vector significantly reduced blood loss in a dose-dependent manner (1.2×10¹²: 0.6 mg/g, 3.0×10¹²: 0.4 mg/g; Jonckheere-Terpstra test: 1-sided P value<0.001).

To test the toxicology of the construct, Male C57BL/6J mice (n=20/group) were intravenously injected with a single bolus dose of 1×10¹³, 3×10¹³, or 5×10¹³ cp/kg vector or formulation buffer (Table 5). Assessment of toxicity was based on clinical signs, body weight, food consumption, ophthalmology, and clinical and anatomical pathology. Complete necropsies were performed on 5 animals from each cohort, and macroscopic findings, organ weights, and the results of microscopic examinations were recorded. Tissues were collected for biodistribution assessment by quantitative polymerase chain reaction from a further 5 animals from each cohort. Blood was collected before dosing and at necropsy. FVIII activity, BDD-FVIII antigen, binding anti-BDD-FVIII antibodies, neutralizing anti-BDD-FVIII antibodies, and binding anti-AAV8 antibodies were analyzed.

TABLE 5
Design of the toxicity study.

| | | Group size | | |
|---|---|---|---|---|
| | Dose | Termination | Termination | Termination |
| Test item | cp/mg | Day 3 | Week 3 | Week 18 |
| Buffer | 0 | 20 | 20 | 20 |
| (controls) | | | | |
| vCS04 | 1 × 10¹³ | 20 | 20 | 20 |
| vCS04 | 3 × 10¹³ | 20 | 20 | 20 |
| vCS04 | 5 × 10¹³ | 20 | 20 | 20 |

It was found that a single intravenous bolus administration of the gene therapy vector at up to 5×10¹³ cp/kg was well tolerated. No deaths occurred during the study and no clinical signs or post-dosing observations were considered to be related to administration of the vector. No negative ophthalmic findings were observed. No effects on body weight or food consumption were observed. No changes in clinical chemistry, hematology, or urinalysis parameters were observed. And no toxicologically relevant macroscopic or microscopic findings were related to the administration of the gene therapy vector.

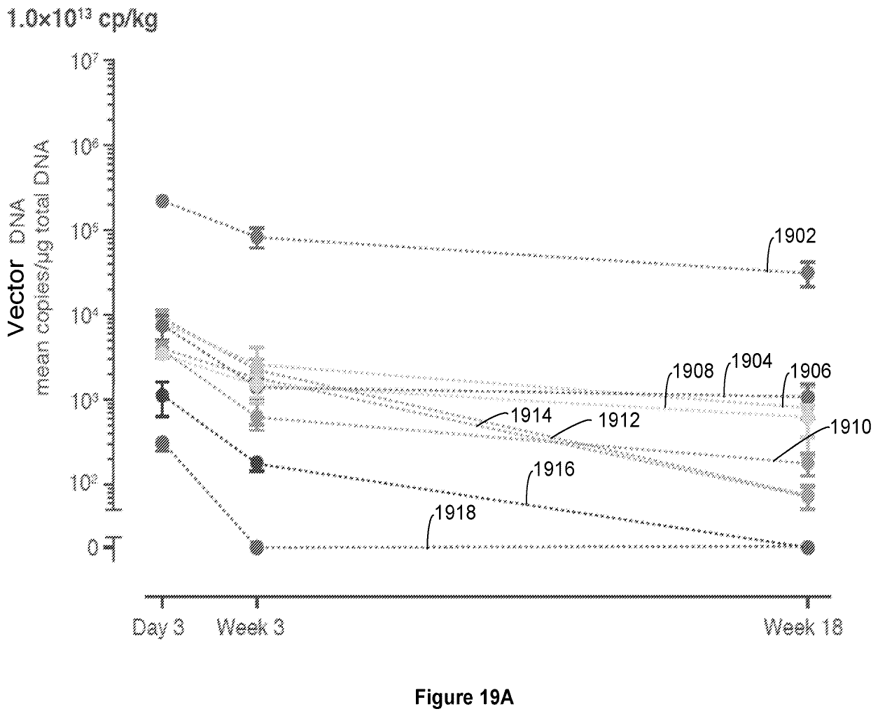

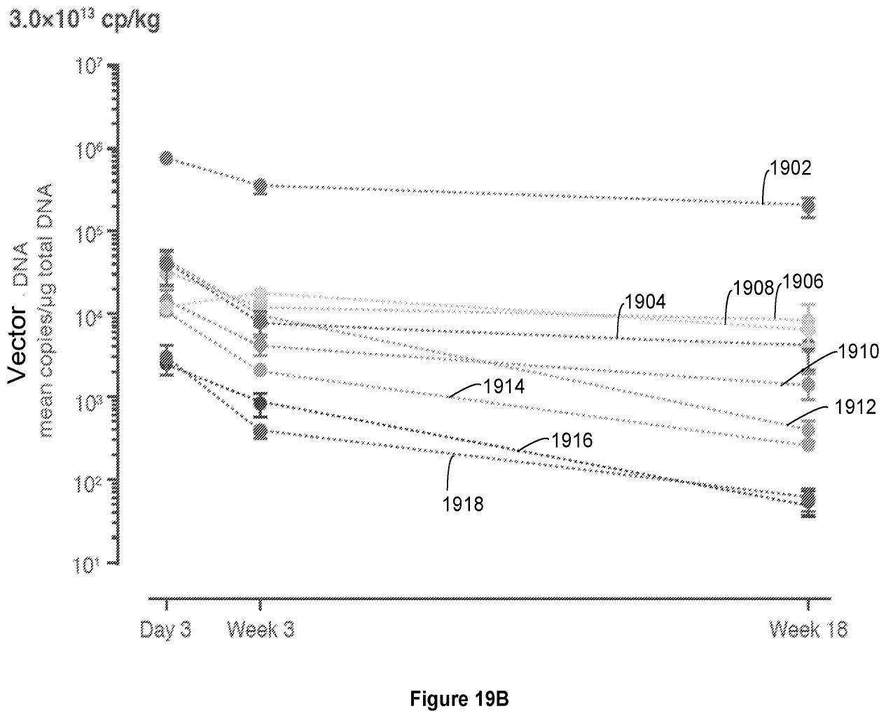

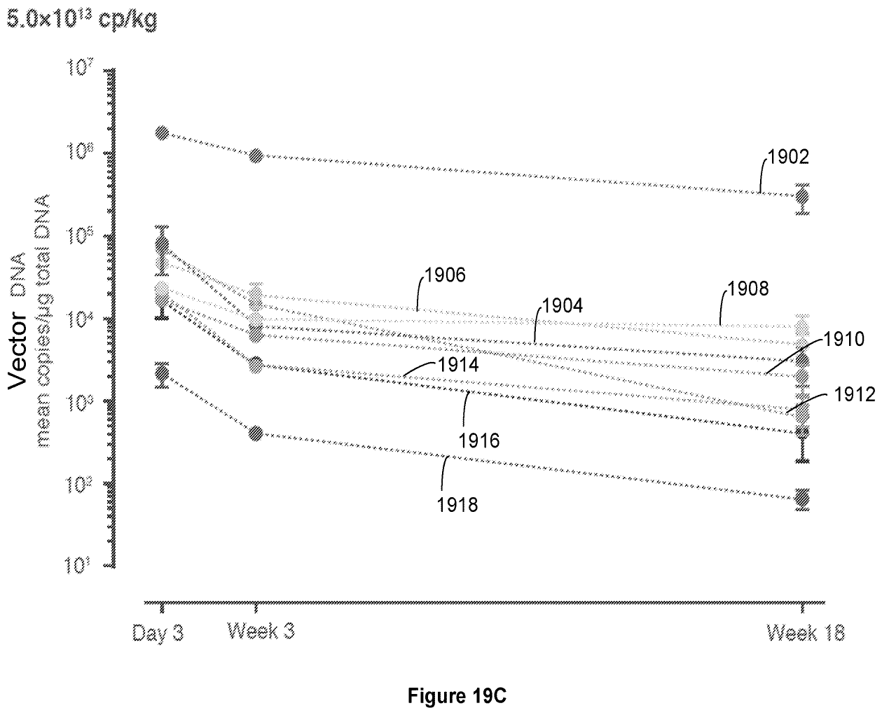

FVIII activity and BDD-FVIII antigen evaluations were prone to wide variability, most likely as a result of the generation of neutralizing antibodies to human BDD-FVIII. However, individual animals in all vector groups had activities above the general baseline levels at Day 3 and Weeks 3 and 18 (data not shown). In the harvested tissue samples, vector DNA was detected predominantly in the liver. Biodistribution to the liver and other tissues was dose related, and was generally highest at the earliest time point, and decreased over time. The presence of vector DNA in brain and testis decreased significantly over time and, in many animals, was below the LLOQ of the assay by Week 18 (FIG. 19).

Taken together, there results show that the codon-optimized BDD-FVII gene therapy is efficacious when administered to FVIII knock-out mice at doses≥1.2×10¹² cp/kg. The no-observed-adverse-effect level was considered to be 5.0×10¹³ cp/kg, the highest dose tested in the toxicity study.

In some embodiments, dosages administered to mice can be converted to human dosages according to the guidance provided in “Guidance for Industry-Estimating the Maximum Safe Starting Dose in Initial Clinical Trials for Therapeutics in Adult Healthy Volunteers,” U.S. Department of Health and Human Services, Food and Drug Administration, Center for Drug Evaluation and Research (CDER), July 2005, Pharmacology and Toxicology, the content of which is hereby incorporated by reference, in its entirety, for all purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for treating hemophilia A comprising intravenously infusing, to a human subject diagnosed with hemophilia A, a dose of 2×10¹² adeno-associated virus (AAV) particles per kilogram body weight of the human subject, wherein the AAV particles comprise a polynucleotide comprising a nucleic acid sequence of SEQ ID NO: 1 (CS04-FL-NA).

2. A method for treating hemophilia A comprising intravenously infusing, to a human subject diagnosed with hemophilia A, a dose of 6×10¹² adeno-associated virus (AAV) particles per kilogram body weight of the human subject, wherein the AAV particles comprise a polynucleotide comprising a nucleic acid sequence of SEQ ID NO: 1 (CS04-FL-NA).

3. The method according to claim 1 or 2, further comprising administering, to the human subject diagnosed with hemophilia A, a course of prednisolone or prednisone.

4. The method of claim 3, wherein said course of prednisolone or prednisone is administered following infusion of the AAV particles.

5. The method of claim 3 or 4, wherein administering the course of prednisolone or prednisone comprises:
administering 60 mg of prednisolone or prednisone per day to the human subject, during the first week immediately following infusion of the AAV particles;
administering 40 mg of prednisolone or prednisone per day to the human subject, during the second week immediately following infusion of the AAV particles; and
administering 30 mg of prednisolone or prednisone per day to the human subject, during the third week immediately following infusion of the AAV particles.

6. The method of claim 5, further comprising administering a tapering dose of prednisolone or prednisone after the third week immediately following infusion of the AAV particles.

7. The method of claim 6, wherein administering the tapering dose of prednisolone or prednisone comprises:
administering 20 mg of prednisolone or prednisone per day to the human subject, for 5 consecutive days immediately following completion of the initial course of prednisolone or prednisone;
administering 15 mg of prednisolone or prednisone per day to the human subject, for 3 consecutive days immediately following the 5 days on which the human subject was administered 20 mg of prednisolone or prednisone;
administering 10 mg of prednisolone or prednisone per day to the human subject, for 3 consecutive days immediately following the 3 days on which the human subject was administered 15 mg of prednisolone or prednisone; and
administering 5 mg of prednisolone or prednisone per day to the human subject, for 3 consecutive days immediately following the 3 days on which the human subject was administered 10 mg of prednisolone or prednisone.

8. The method of claim 6, wherein administering the tapering dose of prednisolone or prednisone comprises:
administering 30 mg of prednisolone or prednisone per day to the human subject, for 7 consecutive days immediately following completion of the initial course of prednisolone or prednisone;
administering 20 mg of prednisolone or prednisone per day to the human subject, for 7 consecutive days immediately following the 7 days on which the human subject was administered 30 mg of prednisolone or prednisone;
administering 15 mg of prednisolone or prednisone per day to the human subject, for 5 consecutive days immediately following the 7 days on which the human subject was administered 20 mg of prednisolone or prednisone;
administering 10 mg of prednisolone or prednisone per day to the human subject, for 5 consecutive days immediately following the 5 days on which the human subject was administered 15 mg of prednisolone or prednisone; and
administering 5 mg of prednisolone or prednisone per day to the human subject, for 5 consecutive days immediately following the 5 days on which the human subject was administered 10 mg of prednisolone or prednisone.

9. A method comprising:
determining a first level of Factor VIII activity in a blood sample collected from a human subject diagnosed with Hemophilia A following administration of adeno-associated virus (AAV) particles comprising a polynucleotide encoding a Factor VIII protein to the human subject, and while the human subject is receiving an initial course of glucocorticoid steroid treatment;
determining a second level of Factor VIII activity in a blood sample collected from the human subject after completion of the initial course of glucocorticoid steroid treatment;
comparing the second level of Factor VIII activity to the first level of Factor VIII activity; and
administering a tapering dose of the glucocorticoid steroid, wherein:
when the second level of Factor VIII activity is not less than the first level of Factor VIII activity, a first tapering dose of the glucocorticoid steroid is administered over a time period of no more than three weeks; and
when the second level of Factor VIII activity is less than the first level of Factor VIII activity, a second tapering dose of the glucocorticoid steroid is administered over a time period exceeding three weeks.

10. A method comprising:
determining a first level of liver enzyme activity in a blood sample collected from a human subject diagnosed with Hemophilia A prior to administration of adeno-associated virus (AAV) particles comprising a polynucleotide encoding a Factor VIII protein to the human subject;
determining a second level of liver enzyme activity in a blood sample collected from the human subject after administration of AAV particles comprising a polynucleotide encoding a Factor VIII protein to the human, and after completion of an initial course of glucocorticoid steroid treatment;
comparing the second level of liver enzyme activity to the first level of liver enzyme activity; and
administering a tapering dose of the glucocorticoid steroid, wherein:
when the second level of liver enzyme activity is not more than the first level of liver enzyme activity, a first tapering dose of the glucocorticoid steroid is administered over a time period of no more than three weeks; and
when the second level of liver enzyme activity is greater than the first level of Factor VIII activity, a second tapering dose of the glucocorticoid steroid is administered over a time period exceeding three weeks.

11. The method of claim 9 or 10, wherein administering the first tapering dose of the glucocorticoid steroid comprises:
administering 20 mg of prednisolone or prednisone per day to the human subject, for 5 consecutive days immediately following completion of the initial course of glucocorticoid steroid treatment;
administering 15 mg of prednisolone or prednisone per day to the human subject, for 3 consecutive days immediately following the 5 days on which the human subject was administered 20 mg of prednisolone or prednisone;
administering 10 mg of prednisolone or prednisone per day to the human subject, for 3 consecutive days immediately following the 3 days on which the human subject was administered 15 mg of prednisolone or prednisone; and
administering 5 mg of prednisolone or prednisone per day to the human subject, for 3 consecutive days immediately following the 3 days on which the human subject was administered 10 mg of prednisolone or prednisone.

12. The method according to any one of claims 9 to 11, wherein administering the second tapering dose of the glucocorticoid steroid comprises:
administering 30 mg of prednisolone or prednisone per day to the human subject, for 7 consecutive days immediately following completion of the initial course of glucocorticoid steroid treatment;
administering 20 mg of prednisolone or prednisone per day to the human subject, for 7 consecutive days immediately following the 7 days on which the human subject was administered 30 mg of prednisolone or prednisone;
administering 15 mg of prednisolone or prednisone per day to the human subject, for 5 consecutive days immediately following the 7 days on which the human subject was administered 20 mg of prednisolone or prednisone;
administering 10 mg of prednisolone or prednisone per day to the human subject, for 5 consecutive days immediately following the 5 days on which the human subject was administered 15 mg of prednisolone or prednisone; and
administering 5 mg of prednisolone or prednisone per day to the human subject, for 5 consecutive days immediately following the 5 days on which the human subject was administered 10 mg of prednisolone or prednisone.

13. A method for monitoring the efficacy of Factor VIII gene therapy of hemophilia A using adeno-associated virus (AAV) particles comprising a polynucleotide encoding a Factor VIII polypeptide, the method comprising:
determining whether Factor VIII inhibitor antibodies are present in a blood sample collected from the human subject after administration of the AAV particles to the human subject; and
upon detecting the presence of a Factor VIII inhibitor in the blood of the human subject, administering an alternative agent for treatment of hemophilia A to the human subject.

14. The method of claim 13, wherein the alternative agent comprises a chemically modified, human Factor VIII protein.

15. The method of claim 13, wherein the alternative agent comprises a porcine Factor VIII protein

16. The method of claim 13, wherein the alternative agent is a Factor VIII bypass therapeutic agent comprising Factor II, Factor IX, and Factor X.

17. A method comprising:
a) administering to a hemophilia A patient a dose of 2×10¹² adeno-associated virus (AAV) particles per kilogram body weight of said patient, wherein the AAV particles comprise a polynucleotide comprising a nucleic acid sequence of SEQ ID NO: 1 (CS04-FL-NA) at a first time point; and
b) measuring the level of SEQ ID NO: 1 or a fragment thereof in said patient's blood stream at a later time point, wherein said later time point is 7 days or longer.

18. A method comprising:
a) administering to a hemophilia A patient a dose of 6×10¹² adeno-associated virus (AAV) particles per kilogram body weight of said patient, wherein the AAV particles comprise a polynucleotide comprising a nucleic acid sequence of SEQ ID NO: 1 (CS04-FL-NA) at a first time point; and
b) measuring the level of SEQ ID NO: 1 or a fragment thereof in said patient's blood stream at a later time point, wherein said later time point is 7 days or longer.

19. A method comprising:
a) administering to a hemophilia A patient a dose of adeno-associated virus (AAV) particles, wherein the AAV particles comprise a polynucleotide encoding a Factor VIII protein at a first time point; and
b) measuring the level of polynucleotide encoding the Factor VIII protein or a fragment thereof in said patient's blood stream at a later time point, wherein said later time point is 7 days or longer.

20. A method according to any one of claims 17 to 19, wherein said later time point is at 7 days.

21. A method according to any one of claims 17 to 19, wherein said later time point is at 14 days.

22. A method according to any one of claims 17 to 19, wherein said later time point is at 21 days.

23. A method comprising:
a) administering to a hemophilia A patient a dose of 2×10¹² adeno-associated virus (AAV) particles per kilogram body weight of said patient, wherein the AAV particles comprise a capsid protein and a polynucleotide comprising a nucleic acid sequence of SEQ ID NO: 1 (CS04-FL-NA) at a first time point; and
b) measuring the level of said capsid protein in said patient's blood stream at a later time point, wherein said later time point is 7 days or longer.

24. A method comprising:
a) administering to a hemophilia A patient a dose of 6×10¹² adeno-associated virus (AAV) particles per kilogram body weight of said patient, wherein the AAV particles comprise a capsid protein and a polynucleotide comprising a nucleic acid sequence of SEQ ID NO: 1 (CS04-FL-NA) at a first time point; and
b) measuring the level of said capsid protein in said patient's blood stream at a later time point, wherein said later time point is 7 days or longer.

25. A method comprising:
a) administering to a hemophilia A patient a dose of adeno-associated virus (AAV) particles, wherein the AAV particles comprise a capsid protein and a polynucleotide encoding a Factor VIII protein at a first time point; and
b) measuring the level of said capsid protein in said patient's blood stream at a later time point, wherein said later time point is 7 days or longer.

26. A method according to any one of claims 23 to 25, wherein said later time point is at 7 days.

27. A method according to any one of claims 23 to 25, wherein said later time point is at 14 days.

28. A method according to any one of claims 23 to 25, wherein said later time point is at 21 days.

29. A method comprising:
a) administering to a hemophilia A patient a dose of 2×10¹² adeno-associated virus (AAV) particles per kilogram body weight of said patient, wherein the AAV particles comprise a polynucleotide comprising a nucleic acid sequence of SEQ ID NO: 1 (CS04-FL-NA) at a first time point; and
b) measuring the level of anti-Factor VIII antibodies in said patient's blood stream at a later time point, wherein said later time point is 7 days or longer.

30. A method comprising:
a) administering to a hemophilia A patient a dose of 6×10¹² adeno-associated virus (AAV) particles per kilogram body weight of said patient, wherein the AAV particles comprise a polynucleotide comprising a nucleic acid sequence of SEQ ID NO: 1 (CS04-FL-NA) at a first time point; and
b) measuring the level of anti-Factor VIII antibodies in said patient's blood stream at a later time point, wherein said later time point is 7 days or longer.

31. A method comprising:
a) administering to a hemophilia A patient a dose of adeno-associated virus (AAV) particles, wherein the AAV particles comprise a polynucleotide encoding a Factor VIII protein at a first time point; and
b) measuring the level of anti-Factor VIII antibodies in said patient's blood stream at a later time point, wherein said later time point is 7 days or longer.

32 A method according to any one of claims 29 to 31, wherein said later time point is at 7 days.

33. A method according to any one of claims 29 to 31, wherein said later time point is at 14 days.

34. A method according to any one of claims 29 to 31, wherein said later time point is at 21 days.

35. A method comprising:
a) administering to a hemophilia A patient a dose of 2×10¹² adeno-associated virus (AAV) particles per kilogram body weight of said patient, wherein the AAV particles comprise a capsid protein and a polynucleotide comprising a nucleic acid sequence of SEQ ID NO: 1 (CS04-FL-NA) at a first time point; and
b) measuring the level of anti-capsid protein antibodies in said patient's blood stream at a later time point, wherein said later time point is 7 days or longer.

36. A method comprising:
a) administering to a hemophilia A patient a dose of 6×10¹² adeno-associated virus (AAV) particles per kilogram body weight of said patient, wherein the AAV particles comprise a capsid protein and a polynucleotide comprising a nucleic acid sequence of SEQ ID NO: 1 (CS04-FL-NA) at a first time point; and
b) measuring the level of anti-capsid protein antibodies in said patient's blood stream at a later time point, wherein said later time point is 7 days or longer.

37. A method comprising:
a) administering to a hemophilia A patient a dose of adeno-associated virus (AAV) particles, wherein the AAV particles comprise a capsid protein and a polynucleotide encoding a Factor VIII protein at a first time point; and
b) measuring the level of anti-capsid protein antibodies in said patient's blood stream at a later time point, wherein said later time point is 7 days or longer.

38. A method according to any one of claims 35 to 37, further comprising:
c) prior to administering the dose of the adeno-associated virus (AAV) particles to said patient, measuring a baseline level of anti-capsid protein antibodies in said patient's blood stream; and
d) optionally, comparing the measured level of anti-capsid protein antibodies in said patient's blood stream at the later time point with the baseline level of anti-capsid protein antibodies in said patient's blood stream.

39. A method according to any one of claims 35 to 38, wherein said later time point is at 7 days.

40. A method according to any one of claims 35 to 38, wherein said later time point is at 14 days.

41. A method according to any one of claims 35 to 38, wherein said later time point is at 21 days.


SEQUENCE LISTING (15 sequences)

SEQ ID NO: 1 - DNA, 4374 bp, synthetic construct
  misc_feature 1..4374: note = Description of Artificial Sequence: Synthetic polynucleotide
  source 1..4374: mol_type = other DNA
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccaggagcttcagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 2 - protein, 1457 aa, synthetic construct
  REGION 1..1457: note = Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 2220 bp, synthetic construct
  misc_feature 1..2220: note = Description of Artificial Sequence: Synthetic polynucleotide
  source 1..2220: mol_type = other DNA
gccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccagg

SEQ ID NO: 4 - DNA, 2052 bp, synthetic construct
  misc_feature 1..2052: note = Description of Artificial Sequence: Synthetic polynucleotide
  source 1..2052: mol_type = other DNA
gagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtac

SEQ ID NO: 5 - DNA, 42 bp, synthetic construct
  misc_feature 1..42: note = Description of Artificial Sequence: Synthetic oligonucleotide
  source 1..42: mol_type = other DNA
agcttcagccagaatccacctgtcctgaaacgccaccagagg

SEQ ID NO: 6 - DNA, 7827 bp, synthetic construct
  misc_feature 1..7827: note = Description of Artificial Sequence: Synthetic polynucleotide
  source 1..7827: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcctcgagatttaaatgacgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactaggcgcgccaccgccaccatgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccaggagcttcagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactgatgacgagcggccgctcttagtagcagtatcgataataaaagatctttattttcattagatctgtgtgttggttttttgtgtgttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagacgatttaaatgacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 4374 bp, synthetic construct
  misc_feature 1..4374: note = Description of Artificial Sequence: Synthetic polynucleotide
  source 1..4374: mol_type = other DNA
atgcagatcgaactgagcacttgcttcttcctgtgtctcctgcgcttttgcttctccgccacaaggagatactatctcggtgccgtggagctcagctgggactacatgcagagcgacttgggtgaactgcctgtggacgccaggtttccaccccgcgtgcccaagagtttcccgttcaacaccagtgtcgtgtacaagaaaaccctcttcgtggaattcaccgaccacctgttcaacatcgccaaaccgcgccctccctggatggggctgctcggcccgacgatccaggctgaggtctatgacacggtggtgattaccctcaagaacatggctagccacccggtgagcctgcacgccgtgggcgtgtcctattggaaagcgtccgagggtgcggagtacgatgaccagacttcacagcgggagaaggaagacgacaaagtgttccccgggggttcccacacctatgtctggcaggtcctgaaggagaatggtcctatggcctccgacccattgtgcctcacctactcttacctaagccatgtggatctcgtcaaggacctgaactcggggctgatcggcgccctgctcgtgtgccgggagggctcactggccaaggagaagacccaaactctgcacaagttcatcctgctgttcgcggtattcgacgaggggaagtcctggcactccgagaccaagaacagcctgatgcaggaccgcgacgcagcctcggcccgtgcgtggccaaagatgcacaccgtgaacggctacgttaacaggagcctacccggcctgatcggctgccaccgcaaatcggtctactggcatgtgatcggaatgggcacaacgcccgaggtccacagtatcttcctcgagggccacactttcctggtccggaatcaccgccaggccagcctggagatcagccccataacctttctgacggcgcagaccttactcatggatctcggccagttcctcctgttctgccacatttcgtcccaccagcacgatgggatggaagcatatgtgaaagtggactcctgccccgaggaaccccagcttaggatgaagaacaatgaggaggccgaggactacgacgatgaccttaccgattcagaaatggacgtagtacgctttgacgacgacaactctccatccttcatacagattcgctccgtcgccaagaagcaccctaagacttgggtgcactacatcgcggccgaggaggaggactgggattatgctcccctggtgctggcccccgacgaccgcagctacaagagccagtacctgaataacgggccccagcgcatcggccggaagtacaagaaagtgcggttcatggcttacacggacgagaccttcaagacccgggaggctatccagcatgagagcggcatcttggggcccctcctgtacggcgaagttggagacacactgctgatcatcttcaagaaccaggcgagcaggccctacaacatctacccccacggcattaccgatgtccggccgttgtacagccgacggctgcccaagggcgtgaagcacctgaaggactttccgatcctgccgggcgagatcttcaagtacaagtggactgtgaccgtggaggatgggccgaccaagagcgatccgcgctgcctgacccgttactactccagctttgtcaatatggagcgcgacctcgctagcggcttgattggccctctgctgatctgctacaaggagtccgtggaccagagggggaatcagatcatgagtgacaagaggaacgtgatcctgttctccgtgttcgacgaaaaccgcagctggtatctcaccgagaatatccagcgcttcctgcccaacccggccggtgtgcagctggaggaccccgagtttcaggccagcaacatcatgcattctatcaacggatatgtgtttgattccctgcagctctcagtgtgtctgcacgaggtcgcctactggtatatcctcagcattggggcacagaccgacttcctgagcgtgttcttctccgggtataccttcaagcacaagatggtgtacgaggataccctgaccctgttcccctttagcggcgaaaccgtgtttatgtctatggagaaccccgggctctggatccttggctgccataactccgacttccgcaaccgcggaatgaccgcgctcctgaaagtgtcgagttgtgacaagaacaccggcgactattacgaggacagttacgaggacatctctgcgtacctccttagcaagaataacgccatcgagccaagatccttcagccagaaccccccagtgctgaagaggcatcagcgggagatcacccgcacgaccctgcagtcggatcaggaggagattgattacgacgacacgatcagtgtggagatgaagaaggaggacttcgacatctacgacgaagatgaaaaccagtcccctcggtccttccaaaagaagacccggcactacttcatcgccgctgtggaacgcctgtgggactatggaatgtcttctagccctcacgttttgaggaaccgcgcccagtcgggcagcgtgccccagttcaagaaagtggtgttccaggagttcaccgacggctccttcacccagccactttaccggggcgagctcaatgaacatctgggcctgctgggaccctacatcagggctgaggtggaggacaacatcatggtgacattccggaatcaggccagcagaccatacagtttctacagttcactcatctcctacgaggaggaccagcgccagggggctgaaccccgtaagaacttcgtgaagccaaacgaaacaaagacctacttctggaaggtccagcaccacatggcacctaccaaggacgagttcgattgcaaggcctgggcctacttctccgacgtggacctggagaaagatgtgcacagcggcctgattggccctctgctggtgtgtcacacgaacacactcaaccctgcacacgggcggcaggtcactgtgcaggaattcgccctgttctttaccatctttgatgagacgaagtcctggtatttcaccgaaaacatggagaggaactgccgcgcaccctgcaacatccagatggaagatccgacattcaaggagaactaccggttccatgccatcaatggctacatcatggacaccctgcctggcctcgtgatggcccaagaccagcgtatccgctggtatctgctgtcgatgggctccaacgagaacatccatagtatccacttcagcgggcatgtcttcacggtgaggaaaaaggaggagtacaagatggcactgtacaacctctatcccggcgtgttcgagaccgtggagatgctgccctccaaggccggcatctggagagtggaatgcctgatcggcgagcacctccacgctgggatgtccacgctgttcctcgtttacagcaataagtgccagacccctctgggcatggcgagcggccacatccgcgacttccagattacagccagcggccagtacggtcagtgggctccaaagctggcccgtctgcactactccggatccatcaacgcctggtccaccaaggaaccgttctcctggatcaaagtagacctgctagcccccatgatcattcacggcatcaagacacaaggcgcccgacagaagttctcgagcctctatatctcccagttcatcatcatgtatagcctggacggaaagaagtggcagacttaccgcggaaactcgacagggaccctgatggtattcttcggtaacgtggacagctccggaatcaagcacaacatcttcaacccacccattatcgcccgctacatccgcctgcaccccactcactatagcattaggtccaccctgcgaatggagctcatgggctgtgacctgaacagctgtagcatgcccctcggcatggagtctaaggcgatctccgacgcacagataacggcatcatcctactttaccaacatgttcgctacctggtccccctccaaggcccgactccacctgcaagggagatccaacgcctggcggccacaggtcaacaatcccaaggagtggctgcaagtggactttcagaaaactatgaaagtcaccggagtgaccacacagggagtgaagtctctgctgaccagcatgtacgtgaaggagttcctcatctccagttcgcaggatggccaccagtggacgttgttcttccaaaacggtaaagtcaaagtcttccaagggaaccaggacagctttacacccgtcgtgaactccctggaccccccgcttctcactagatacctccgcatccaccctcagagctgggtgcaccagattgccctgcgcatggaggttctggggtgtgaagcccaggacctgtactaa

SEQ ID NO: 8 - DNA, 4374 bp, synthetic construct
  misc_feature 1..4374: note = Description of Artificial Sequence: Synthetic polynucleotide
  source 1..4374: mol_type = other DNA
atgcagattgagctctccacctgcttctttctctgccttcttcgcttctgcttttctgccacacgcaggtactatttgggagcagtggaactgagctgggattacatgcagagtgaccttggtgaacttcctgtggacgctcgttttccacctagagttcccaagtccttccccttcaacacctcagtggtctacaagaaaacgctgtttgtggagttcactgaccacctcttcaacattgccaaaccaagacccccttggatgggattgctgggacccacaatacaagcagaagtctacgacacggtggtgattaccctgaagaacatggcgtcacaccctgtttcacttcacgctgttggggtcagttattggaaagcctcagagggtgcggaatacgatgatcaaaccagccagagggagaaggaagatgacaaggtctttcctgggggtagccatacctatgtttggcaggtgctgaaagagaatgggcctatggcctctgatcccttgtgcctcacatactcttacctgagtcacgtcgacctggtgaaagacctgaatagcggtctgattggtgcactgcttgtttgtagagaggggagtttggccaaggagaaaactcagactctccacaagtttatcctcctgtttgctgtgttcgacgagggcaagtcttggcactctgaaacaaagaactccctgatgcaggacagagatgctgcatctgcaagggcttggccaaaaatgcacacagtgaacggctatgtgaatcgatcactgccaggactgataggctgtcatcgcaagtcagtgtattggcacgttatcgggatgggaacaactccagaagtgcacagcatcttccttgagggccacactttcctggttcggaatcatagacaggccagccttgagatcagcccaatcacctttctgactgcccaaaccttgctgatggatctgggacagttcctcctgttttgtcacatctcctcccaccaacatgacgggatggaggcttatgtgaaggtcgatagctgtccggaggaaccacaactgaggatgaagaacaacgaagaggcagaggactatgacgacgatctgactgacagtgaaatggacgtggttcggttcgacgatgacaattctccttcatttatccagatccgttccgtggccaagaagcaccccaagacttgggttcattacatcgctgctgaggaggaggattgggactacgcgcccttggtgttggccccagacgatcgctcatacaagagccagtaccttaacaatggtccacaaaggatcggccggaagtacaagaaggttagatttatggcttataccgacgagacttttaaaactagggaagcaattcagcatgaaagtggcattcttggacccctgctgtatggcgaggttggcgacaccctgctgattatctttaagaaccaggcaagccggccctacaacatctacccgcacggcataaccgatgtacgacccctgtacagtcgcagacttcctaaaggggtgaaacacctgaaggacttcccaattctgcccggggagatcttcaagtataaatggaccgtgacggttgaggatggtcccacaaagtccgatccgagatgccttacccgatattattccagcttcgtgaacatggaaagggacctggccagcgggctgattggcccactgctgatttgttacaaggagtctgtcgatcaaagaggaaaccaaataatgagcgacaaacgtaacgtcatcctgttcagcgtctttgatgagaatagaagctggtacctcacagaaaatattcagcggtttctgcctaaccccgcaggcgtccagctggaagatcccgagttccaagcctcaaacatcatgcatagcatcaacggatacgtattcgatagcctgcagctgtccgtctgtctccatgaagtggcatattggtacatcctgagtatcggggcgcagaccgacttcctgagcgtgttcttttctggatacacgttcaaacacaaaatggtctatgaagataccctgactctgtttccattctcaggagagacagtctttatgagtatggaaaatcctggactgtggatcctgggctgtcacaattctgattttcggaacagaggcatgacagccctgcttaaagtgagctcatgcgacaagaacaccggtgattactacgaagatagctatgaggacatcagtgcgtatttgctctccaagaacaacgctatcgagccacggtctttcagtcagaatcctcccgttctgaagcggcatcagcgcgaaataacacgcacaacccttcagtcagaccaagaggaaatcgactacgatgatactatctctgtggagatgaagaaggaggatttcgacatttacgacgaggacgagaatcagtccccaaggagctttcagaagaaaacaagacactatttcattgccgccgtggagcgactgtgggactacggcatgtctagctctccgcatgtacttagaaatagggcacaaagcggatccgtgcctcagtttaagaaagttgtctttcaggagtttacagatggctccttcacccagcccttgtatcgcggggaactcaatgaacacctgggcctcctgggtccttatattagggccgaagtcgaggacaatatcatggtgacctttaggaaccaggcatctagaccttactctttctactcctccctgatatcctatgaggaggaccagcggcaaggcgctgagcctcggaagaactttgtgaagccaaatgaaaccaaaacatacttttggaaagttcagcaccacatggctcccacgaaggacgaatttgactgtaaagcctgggcctacttctcagatgtagatctcgagaaagacgtgcactcagggctcattggtcccctcctggtctgtcatactaataccctcaatccagcacacggacgtcaggtaaccgtccaggaatttgccctgttctttaccattttcgatgagactaaatcctggtactttaccgaaaacatggagaggaattgcagagccccatgcaacatccagatggaggaccctaccttcaaagagaactatcgcttccatgccattaacggttacattatggatactctcccaggacttgtgatggcacaggatcagcggataagatggtatctgttgagcatgggctccaacgagaatattcacagcatccatttctccggtcacgtgtttacagtgagaaagaaagaagagtacaagatggctctgtataatctctatccaggcgtattcgaaacggtggagatgttgcctagcaaggccggcatttggcgagtagaatgccttatcggggaacatctgcatgccggaatgagcacgctcttcctggtgtatagtaacaagtgccagactccgctgggcatggcatctggccatatacgggactttcagattacggctagcgggcagtatgggcagtgggcacccaaacttgcgcgactgcactattcaggctctatcaatgcatggtccaccaaggaacccttctcttggattaaggtggaccttttggcgcccatgataatccatgggatcaaaacccagggcgctcgtcagaaattctcatcactctacatctctcagttcataataatgtattcactggatgggaagaaatggcagacttacagaggaaacagcaccgggacgctgatggtgttctttggcaacgtggacagcagcggcatcaaacacaacatcttcaatcctcccattattgcccgttatattagactgcatcccactcactactctatacgcagcacacttaggatggagctcatgggatgcgacctgaacagttgtagtatgcccttggggatggagtccaaagctataagcgacgcacaaattacagctagctcttactttacgaatatgttcgccacgtggagcccaagcaaagcccggctgcatttgcagggtcggagtaatgcttggcgcccacaggtgaataaccctaaggaatggttgcaagtagatttccagaaaactatgaaggtaaccggcgtcactacacagggagtcaagtccctcttgacctctatgtacgtcaaggagttcctgattagcagcagtcaggatgggcaccaatggacactgttcttccagaatgggaaagttaaagtatttcagggtaaccaggactcctttacacctgtggtgaatagcctcgacccacccctgctgacacgatacctccgcatccaccctcagtcttgggtgcatcaaattgccctgcgaatggaggtgttgggatgcgaagctcaggacctctactga

SEQ ID NO: 9 - DNA, 4374 bp, synthetic construct
  misc_feature 1..4374: note = Description of Artificial Sequence: Synthetic polynucleotide
  source 1..4374: mol_type = other DNA
atgcagatcgaactctctacttgcttcttcctgtgccttctgaggttctgcttctctgccactcgccgatattacctcggggccgtggagttgagttgggactacatgcaatcagatctgggcgaactccctgtggatgcccgattcccaccgcgcgtgcccaagtctttcccatttaatacttctgtggtgtacaagaagacattgtttgtggagtttaccgatcacctgttcaacatcgccaaaccgcggcccccatggatgggtctgcttgggcccaccattcaagcggaggtctatgatacagtggtgataacgcttaagaacatggcgagccacccagtgtctctgcatgccgttggtgtatcatattggaaggccagcgaaggagcggagtacgatgaccagacctctcagagagagaaggaagacgataaggtttttcctggcggaagtcatacatatgtatggcaggtcctgaaagagaatgggccgatggcttctgaccccctttgtcttacctatagttatctgagccacgtggacctggtcaaggacctcaacagtggtctgattggggctctgcttgtttgtagagagggtagcttggctaaggagaaaacccaaacactccataagttcattttgctgttcgcggtgttcgacgagggaaagagttggcacagcgaaacaaagaattcactgatgcaagacagggacgccgcttccgcaagggcttggcctaagatgcatacggtgaatgggtatgtgaaccggagcctcccggggctgatcgggtgccatcgcaagtctgtttactggcacgtcattggaatggggacaacgccagaggtacatagtatatttcttgaaggccacacgttcctcgtacggaaccaccgacaggcttccctggagataagccccattacctttctgaccgctcagactctgctgatggaccttggccagtttctcctgttctgccatattagcagccaccagcacgacggtatggaagcatacgtgaaagtcgatagctgtcctgaggagcctcagctcagaatgaagaacaacgaggaggccgaagactatgacgatgaccttacagattccgagatggacgtggtgcgctttgacgacgataacagtcctagtttcattcaaatcagatccgtagccaaaaagcatccaaagacatgggtgcattacattgcagccgaagaggaggattgggattatgcgccccttgttctggctccagatgacaggagctataagtcccagtacttgaacaacgggccacagcgaatcggtagaaaatataagaaggtaagattcatggcctacactgacgaaacatttaaaaccagggaagctatccaacacgaatctggaattctcggccctctgctctacggtgaggtgggggacaccttgctgatcattttcaaaaatcaggcatccaggccttacaacatatacccccatggcatcaccgatgtccgcccgctgtattccagaagactccccaagggagtgaaacatctgaaagattttcccatcctgccgggcgagatctttaaatacaaatggactgtgactgtagaggacgggcctacaaaatcagacccacggtgcctgacaaggtattacagtagcttcgtcaacatggaacgcgacctcgccagcggactcattggcccactgttgatctgttacaaagagtcagtggatcagaggggaaatcagatcatgagcgataagagaaacgttatcctgtttagtgtcttcgacgagaaccggtcttggtaccttactgagaacatccagaggttcctgccgaatccggctggcgttcagctcgaggacccagagttccaggccagtaatataatgcactcaatcaacggttatgtgttcgatagcctgcagctgagcgtctgcctccacgaggtagcctattggtacatattgtccatcggggctcagaccgattttctgtccgtgttctttagcgggtatacctttaaacataaaatggtctatgaagacaccctgaccctgttcccattctccggtgagactgtgttcatgtccatggagaacccagggctgtggatcctggggtgtcacaatagtgactttaggaatcggggaatgacggcactgctgaaggtgagttcttgcgataaaaatacaggagattactatgaggatagttacgaggatatcagtgcctatctgctttcaaaaaacaacgcaattgagccccggtctttctcacaaaaccccccggtgctgaagcgccaccagcgcgaaattacccggacaaccttgcagtccgaccaggaggaaatcgattatgacgatactatcagtgtagaaatgaaaaaggaggattttgatatttacgacgaagacgagaaccagtctccgcgaagttttcagaagaaaacgcgacactactttatagctgccgtggaacgactctgggattatggcatgtcctccagccctcatgtccttaggaatcgagcgcagagtggctctgtgcctcagttcaaaaaggttgtgttccaggaattcaccgacggctcatttacccagccgctgtacagaggcgaactcaacgaacaccttgggctgcttgggccatatattcgagcagaggtggaagataatatcatggtaacctttagaaaccaggcgtcaagaccctattccttctacagttctctgatcagctacgaggaggaccaaagacagggagctgaacccaggaagaactttgtgaaacctaatgagaccaagacctacttctggaaggtccagcaccatatggccccaactaaagatgaattcgattgcaaggcctgggcttatttcagcgacgtggatctcgaaaaggatgtgcacagcgggttgatcggaccgcttttggtgtgccacacaaataccctcaatcctgcccacgggcggcaggtcacagttcaagagtttgcactcttctttacaatatttgacgagacaaagtcatggtattttacagagaatatggagagaaattgtcgcgcaccttgcaacattcagatggaggaccccacatttaaggagaattacagatttcatgctatcaatgggtacattatggatactctgcctggtctggtcatggcccaggatcagcgcataaggtggtacttgctgagcatgggatctaatgagaatatacacagcattcacttcagtggccacgtttttactgttagaaagaaggaggagtacaaaatggcgctctacaacctttacccgggtgtgtttgagacagtggagatgctgccaagcaaggcaggcatctggagggttgagtgtcttattggggagcatctgcatgctggaatgtccaccctctttcttgtgtacagcaataagtgccagacaccgcttggcatggccagcggccacattagggactttcagataactgccagtggacagtacggccagtgggctcccaagcttgcaagactccactactccggaagcataaacgcatggagcaccaaggaacccttctcttggattaaggtggacctgctggcgccaatgatcattcacggcataaaaacccaaggggcacgacagaaattttcatctttgtatattagtcagtttatcatcatgtacagcttggatggaaagaagtggcagacgtacaggggcaattctacaggaacacttatggtgttttttgggaatgtcgattccagcgggatcaaacataacatcttcaatcctcctattatcgcccgatatatccgcctgcaccctacgcattactccatcaggtccacattgagaatggaactgatggggtgcgacctgaatagttgtagtatgccactgggcatggagtctaaagccatcagcgatgcacagatcactgccagctcttacttcaccaacatgtttgcaacttggtccccctctaaagctcgcctgcatctgcagggacgctcaaatgcatggcgaccacaggtgaacaatccaaaagagtggctccaggtcgactttcagaagacaatgaaggtaacaggagtgacaacccagggtgtaaaaagcctccttacgagtatgtacgttaaggagtttctgatttctagctcccaggacggacaccagtggactctgttcttccagaacggcaaagtgaaggtatttcagggaaaccaggattcttttaccccggtagtgaatagcctggatccaccgttgctgacccgctatctgagaattcatccacaatcctgggtgcatcagattgccctccggatggaagtgctcggctgtgaagctcaggatctgtattag

SEQ ID NO: 10 - DNA, 4374 bp, synthetic construct
  misc_feature 1..4374: note = Description of Artificial Sequence: Synthetic polynucleotide
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 11 - DNA, 4374 bp, synthetic construct
  misc_feature 1..4374: note = Description of Artificial Sequence: Synthetic polynucleotide
  source 1..4374: mol_type = other DNA
atgcagatcgagctgtccacatgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaacatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggcctccgagggcgccgagtacgacgaccagaccagccagcgggagaaagaggacgacaaagtctttcctggcggcagccacacctacgtgtggcaggtcctgaaagaaaacggccccatggcctccgaccccctgtgcctgacctacagctacctgagccacgtggacctggtgaaggacctgaacagcgggctgattggggccctgctggtctgccgggagggcagcctggccaaagagaaaacccagaccctgcacaagttcatcctgctgttcgccgtgttcgacgagggcaagagctggcacagcgagaccaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtgcggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcctgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactacatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagaccttcaagacccgggaggccatccagcacgagagcggcatcctgggccccctgctgtacggcgaagtgggcgacacactgctgatcatcttcaagaaccaggccagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggggtgcagctggaagatcccgagttccaggccagcaacatcatgcactccatcaatggctacgtgttcgacagcctgcagctgtccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagaccgtgttcatgagcatggaaaaccccggcctgtggatcctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgagcccagaagcttcagccagaacccccctgtgctgaagcggcaccagagagagatcacccggaccaccctgcagtccgaccaggaagagatcgattacgacgacaccatcagcgtggagatgaaaaaagaagatttcgacatctacgacgaggacgagaaccagagcccccggtccttccagaagaaaacccggcactactttatcgccgccgtggagcggctgtgggactacggcatgagcagcagcccccacgtgctgcggaaccgggcccagagcggcagcgtgccccagttcaagaaagtggtgttccaggaattcaccgacggcagcttcacccagcccctgtaccggggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgctgaaccccggaagaacttcgtgaagcccaatgagaccaagacctacttctggaaagtgcagcaccacatggcccccaccaaggacgagttcgactgcaaggcctgggcctacttcagcgacgtggatctggaaaaggacgtgcactctggactgattggccctctgctggtgtgccacaccaacaccctgaaccccgcccacggccggcaggtgaccgtgcaggaattcgccctgttcttcaccatcttcgacgagaccaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagaggatccggtggtatctgctgtccatgggcagcaacgagaatatccacagcatccacttcagcggccacgtgttcaccgtgaggaagaaagaagagtacaagatggccctgtacaacctgtaccccggcgtgttcgagaccgtggagatgctgcccagcaaggccggcatctggcgggtggagtgtctgatcggcgagcacctgcatgccgggatgagcaccctgtttctggtgtacagcaacaagtgccagacccccctgggcatggccagcggccacatccgggacttccagatcaccgcctccggccagtacggccagtgggcccccaagctggcccggctgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaacgtggacagcagcggcatcaagcacaacatcttcaacccccccatcatcgcccggtacatccggctgcaccccacccactacagcatcagatccaccctgcggatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtgaacaaccccaaagaatggctgcaggtggactttcagaaaaccatgaaggtgaccggcgtgaccacccagggcgtgaaaagcctgctgaccagcatgtacgtgaaagagtttctgatcagcagcagccaggacggccaccagtggaccctgttctttcagaacggcaaggtgaaagtgttccagggcaaccaggactccttcacccccgtggtgaactccctggacccccccctgctgacccgctacctgcggatccacccccagtcttgggtgcaccagatcgccctgaggatggaagtgctgggatgtgaggcccaggatctgtactga

SEQ ID NO: 12 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - protein, 14 aa, synthetic construct
  REGION 1..14: note = Description of Artificial Sequence: Synthetic peptide
SFSQNPPVLKRHQR

SEQ ID NO: 14 - protein, 24 aa, Sus sp.
SFAQNSRPPSASAPKPPVLRRHQR

SEQ ID NO: 15 - protein, 16 aa, Sus sp.
SFSQNSRHQAYRYRRG